(19) United States
(12) Patent Application Publication
(10) Pub. No.: US 2024/0238309 A1
(43) Pub. Date: Jul. 18, 2024

(54) PHARMACEUTICAL FORMULATIONS OF TREPROSTINIL PRODRUGS AND METHODS OF USE THEREOF

(71) Applicant: Insmed Incorporated, Bridgewater, NJ (US)

(72) Inventors: Vladimir MALININ, Bridgewater, NJ (US); Adam PLAUNT, Bridgewater, NJ (US)

(73) Assignee: Insmed Incorporated, Bridgewater, NJ (US)

(21) Appl. No.: 17/907,179

(22) PCT Filed: Mar. 25, 2021
(86) PCT No.: PCT/US21/24077

§ 371 Date: Sep. 23, 2022

Related U.S. Application Data
(60) Provisional application No. 62/994,596, filed on Mar. 25, 2020.

(51) Int. Cl. A61K 31/5575; A61K 9/00; A61K 47/10

(52) U.S. Cl. CPC A61K 31/5575; A61K 9/0078; A61K 9/008; A61K 47/10

(57) ABSTRACT

The present disclosure provides pharmaceutical formulations of treprostinil prodrugs and methods for their preparation and use. In and use in treating pulmonary hypertension (e.g., pulmonary arterial hypertension), portopulmonary hypertension, and pulmonary fibrosis by inhalation via a metered dose inhaler. The formulations can include (a) a treprostinil prodrug, e.g., a treprostinil alkyl ester or amide, (b) polyoxyethylene (20) cetylether or at least one polyethylene glycol-lipid (PEGylated lipid), (c) at least one surfactant selected from the group consisting of polyethylene glycol (PEG) and propylene glycol, (d) at least one hydrofluoroalkane propellant, and (e) at least one alcohol cosolvent.

### CROSS REFERENCE TO RELATED APPLICATION

This application claims priority from U.S. Provisional Application Ser. No. 62/994,596, filed Mar. 25, 2020, the disclosure of which is incorporated by reference herein in its entirety.

### BACKGROUND OF THE INVENTION

Pulmonary hypertension (PH) is characterized by an abnormally high blood pressure in the lung vasculature. It is a progressive, lethal disease that leads to heart failure and can occur in the pulmonary artery, pulmonary vein, or pulmonary capillaries. Symptomatically patients experience shortness of breath, dizziness, fainting, and other symptoms, all of which are made worse by exertion. There are multiple causes, and can be of unknown origin, idiopathic, and can lead to hypertension in other systems, for example, portopulmonary hypertension in which patients have both portal and pulmonary hypertension.

Pulmonary hypertension has been classified into five groups by the World Health Organization (WHO). Group 1 is called pulmonary arterial hypertension (PAH), and includes PAH that has no known cause (idiopathic), inherited PAH (i.e., familial PAH or FPAH), PAH that is caused by drugs or toxins, and PAH caused by conditions such as connective tissue diseases, HIV infection, liver disease, and congenital heart disease. Group 2 pulmonary hypertension is characterized as pulmonary hypertension associated with left heart disease. Group 3 pulmonary hypertension is characterized as PH associated with lung diseases, such as chronic obstructive pulmonary disease and interstitial lung diseases, as well as PH associated with sleep-related breathing disorders (e.g., sleep apnea). Group 4 PH is PH due to chronic thrombotic and/or embolic disease, e.g., PH caused by blood clots in the lungs or blood clotting disorders. Group 5 includes PH caused by other disorders or conditions, e.g., blood disorders (e.g., polycythemia vera, essential thrombocythemia), systemic disorders (e.g., sarcoidosis, vasculitis), and metabolic disorders (e.g., thyroid disease, glycogen storage disease).

Pulmonary arterial hypertension (PAH) afflicts approximately 200,000 people globally with approximately 30,000-40,000 of those patients in the United States. PAH patients experience constriction of pulmonary arteries which leads to high pulmonary arterial pressures, making it difficult for the heart to pump blood to the lungs. Patients suffer from shortness of breath and fatigue which often severely limits the ability to perform physical activity.

The New York Heart Association (NYHA) has categorized PAH patients into four functional classes to rate the severity of the disease. Class I PAH patients as categorized by the NYHA do not have a limitation of physical activity, as ordinary physical activity does not cause undue dyspnoea or fatigue, chest pain, or near syncope. Class II PAH patients as categorized by the NYHA have a slight limitation on physical activity. These patients are comfortable at rest, but ordinary physical activity causes undue dyspnoea or fatigue, chest pain or near syncope. Class III PAH patients as categorized by the NYHA have a marked limitation of physical activity. Although comfortable at rest, class III PAH patients experience undue dyspnoea or fatigue, chest pain or near syncope as a result of less than ordinary physical activity. Class IV PAH patients as categorized by the NYHA are unable to carry out any physical activity without symptoms. Class IV PAH patients might experience dyspnoea and/or fatigue at rest, and discomfort is increased by any physical activity. Signs of right heart failure are often manifested by class IV PAH patients.

Patients with PAH are treated with an endothelin receptor antagonist (ERA), phosphodiesterase type 5 (PDE-5) inhibitor, a guanylate cyclase stimulator, a prostanoid (e.g., prostacyclin), or a combination thereof. ERAs include abrisentan (Letairis®), sitaxentan, bosentan (Tracleer®), and macitentan (Opsumit®). PDE-5 inhibitors indicated for the treatment of PAH include sildenafil (Revatio®) and tadalafil (Adcirca®). Prostanoids indicated for the treatment of PAH include iloprost, epoprosentol and treprostinil (Remodulin®, Tyvaso®). The one approved guanylate cyclase stimulator is riociguat (Adempas®). Additionally, patients are often treated with combinations of the aforementioned compounds.

Portopulmonary hypertension (PPH) is defined by the coexistence of portal and pulmonary hypertension, and is a serious complication of liver disease. The diagnosis of portopulmonary hypertension is based on hemodynamic criteria: (1) portal hypertension and/or liver disease (clinical diagnosis-ascites/varices/splenomegaly), (2) mean pulmonary artery pressure>25 mmHg at rest, (3) pulmonary vascular resistance>240 dynes s/cm⁵, (4) pulmonary artery occlusion pressure<15 mmHg or transpulmonary gradient>12 mmHg. PPH is a serious complication of liver disease, and is present in 0.25 to 4% of patients suffering from cirrhosis. Today, PPH is comorbid in 4-6% of those referred for a liver transplant.

Pulmonary fibrosis is a respiratory disease in which scars are formed in the lung tissues, leading to serious breathing problems. Scar formation, i.e., the accumulation of excess fibrous connective tissue, leads to thickening of the walls, and causes reduced oxygen supply in the blood. As a result, pulmonary fibrosis patients suffer from perpetual shortness of breath. In some patients the specific cause of the disease can be diagnosed, but in others the probable cause cannot be determined, a condition called idiopathic pulmonary fibrosis.

The present disclosure provides pharmaceutical formulations of treprostinil prodrugs useful for pulmonary administration to treat pulmonary hypertension (PH) (including pulmonary arterial hypertension (PAH)), portopulmonary hypertension (PPH), and pulmonary fibrosis. The pharmaceutical formulations are suitable for use in metered dose inhalers (MDIs).

### SUMMARY OF THE INVENTION

The present application relates to pharmaceutical formulations suitable for administration by inhalation via a metered dose inhaler (MDI), methods for preparation of the pharmaceutical formulations and their use in therapy.

In one aspect, the present application provides pharmaceutical formulations suitable for administration by inhalation via a metered dose inhaler (MDI). In one embodiment, the pharmaceutical formulation includes (a) a compound of Formula (I):

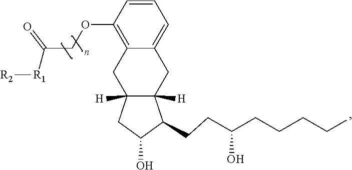

or an enantiomer, diastereomer, or pharmaceutically acceptable salt thereof, wherein R₁ is NH, O or S; R₂ is a linear or branched C₅-C₁₈ alkyl, a linear C₂-C₁₈ alkenyl or a branched C₃-C₁₈ alkenyl, aryl, aryl-C₁-C₁₈ alkyl, an amino acid or a peptide; and n is an integer from 0 to 5;
(b) polyoxyethylene (20) cetylether or at least one polyethylene glycol-lipid (PEGylated lipid);
(c) a surfactant selected from polyethylene glycol (PEG), propylene glycol, or a combination thereof,
(d) at least one hydrofluoroalkane propellant, and
(e) at least one alcohol cosolvent.

In one embodiment, the compound of Formula (I), or an enantiomer, diastereomer, or pharmaceutically acceptable salt thereof, is a compound of Formula (Ia):

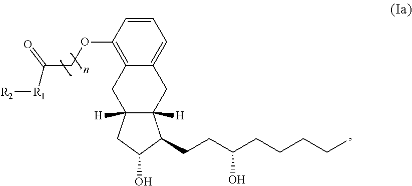

or an enantiomer, diastereomer, or pharmaceutically acceptable salt thereof, wherein R₁ is O; R₂ is dodecyl, tridecyl, tetradecyl, pentadecyl, hexadecyl, heptadecyl or octadecyl; and n is an integer from 0 to 5. In one embodiment, the compound of Formula (I), or pharmaceutically acceptable salt thereof, is a compound of Formula (Ia) or a pharmaceutically acceptable salt thereof. In another embodiment, the compound of Formula (I), or pharmaceutically acceptable salt thereof, is a compound of Formula (Ia). In some embodiments, n is 0 or 1. In one embodiment, n is 0. In another embodiment, n is 1.

In another embodiment, the compound of Formula (I), or an enantiomer, diastereomer, or pharmaceutically acceptable salt thereof, is a compound of Formula (Ib):

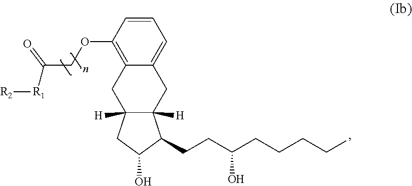

or an enantiomer, diastereomer, or pharmaceutically acceptable salt thereof, wherein n is O, R₁ is NH or O, and R₂ is a linear C₅-C₁₈ alkyl. In one embodiment, the compound of Formula (I), or pharmaceutically acceptable salt thereof, is a compound of Formula (Ib) or a pharmaceutically acceptable salt thereof. In another embodiment, the compound of Formula (I), or pharmaceutically acceptable salt thereof, is a compound of Formula (Ib). In one embodiment, R₁ is NH. In another embodiment, R₁ is O. In some embodiments, R₂ is linear heptyl, linear octyl, linear nonyl, linear decyl, linear undecyl, linear dodecyl, linear tridecyl, linear tetradecyl, linear pentadecyl, linear hexadecyl, linear heptadecyl or linear octadectyl.

In one embodiment, the compound of Formula (I), (Ia), or (Ib), or an enantiomer, diastereomer or pharmaceutically acceptable salt thereof is present at a concentration of from about 0.5 to about 3 mg/mL.

In one embodiment, the polyoxyethylene (20) cetylether is present at a concentration of from about 0.25 to about 0.75 mg/mL, e.g., about 0.5 mg/mL.

In one embodiment, the at least one PEGylated lipid is one PEGylated lipid. The one PEGylated lipid may be selected from the group consisting of DSPE (distearoylphosphatidylethanolamine)-PEG2000, DSG (disteraroylglycerol)-PEG2000, and DPG (diphosphatidylglycerol)-PEG2000. In another embodiment, the at least one PEGylated lipid consists of a double or triple combination of DSPE-PEG2000, DSG-PEG2000, and DPG-PEG2000.

In one embodiment, the at least one PEGylated lipid is present at a concentration of from about 0.2 to about 3 mg/mL.

In one embodiment, the surfactant is one surfactant selected from the group consisting of PEG400, PEG1000, and propylene glycol. In another embodiment, the surfactant consists of a double or triple combination of PEG400, PEG1000, and propylene glycol.

In one embodiment, the surfactant is present at a concentration of from about 0.75 to about 6 mg/mL.

In one embodiment, the at least one hydrofluoroalkane propellant is one hydrofluoroalkane propellant. The one hydrofluoroalkane propellant may be selected from the group consisting of 1,1,1,2-tetrafluoroethane (HFA134a), 1,1,1,2,3,3,3-heptafluoro-n-propane (HFA227ea), and 1,1-difluoroethane (IFA152a). In another embodiment, the at least one hydrofluoroalkane propellant consists of a double or triple combination of HFA134a, HFA227ea, and HFA152a.

In one embodiment, the at least one alcohol cosolvent is one alcohol cosolvent. The one alcohol cosolvent may be selected from the group consisting of ethanol and isopropyl alcohol. In another embodiment, the at least one alcohol cosolvent is a combination of ethanol and isopropyl alcohol.

In one embodiment, the at least one alcohol cosolvent is present at a concentration of from about 3% to about 10% (w/w) based on the total weight of the pharmaceutical formulation.

In another aspect, the present application provides a pharmaceutical formulation suitable for administration by inhalation via a metered dose inhaler (MDI). The pharmaceutical formulation comprises or consists of (a) a compound of Formula (II):

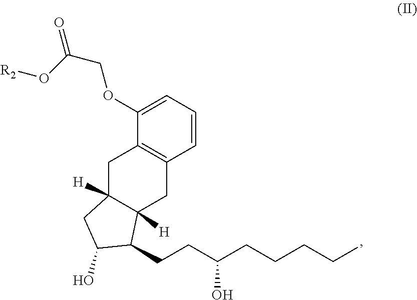

or an enantiomer, diastereomer, or pharmaceutically acceptable salt thereof at a concentration of from about 0.5 to about 3 mg/mL, wherein R₂ is dodecyl, tridecyl, tetradecyl, pentadecyl, hexadecyl, heptadecyl, or octadecyl,
(b) polyoxyethylene (20) cetylether at a concentration of from about 0.25 to about 0.75 mg/mL, or at least one PEGylated lipid selected from the group consisting of DSPE-PEG2000, DSG-PEG2000, and DPG-PEG2000 at a concentration of from about 0.2 to about 3 mg/mL,
(c) a surfactant selected from PEG400, PEG1000, propylene glycol, or a combination thereof at a concentration of from about 0.75 to about 6 mg/mL,
(d) at least one hydrofluoroalkane propellant selected from the group consisting of HFA134a, HFA227ea, and HFA152a, and
(e) at least one alcohol cosolvent selected from the group consisting of ethanol and isopropyl alcohol at a concentration of from about 3% to about 10% (w/w) based on the total weight of the pharmaceutical formulation.

In one embodiment, (a) is a compound of Formula (II) or a pharmaceutically acceptable salt thereof. In another embodiment, (a) is a compound of Formula (II).

In one embodiment, the polyoxyethylene (20) cetylether is present at a concentration of about 0.5 mg/mL.

In one embodiment, the at least one PEGylated lipid is one PEGylated lipid selected from the group consisting of DSPE-PEG2000, DSG-PEG2000, and DPG-PEG2000. In another embodiment, the at least one PEGylated lipid consists of a double or triple combination of DSPE-PEG2000, DSG-PEG2000, and DPG-PEG2000.

In one embodiment, the surfactant is one selected from the group consisting of PEG400, PEG1000, and propylene glycol. In another embodiment, the surfactant consists of a double or triple combination of PEG400, PEG1000, and propylene glycol.

In one embodiment, the at least one hydrofluoroalkane propellant is one hydrofluoroalkane propellant selected from the group consisting of HFA134a, HFA227ea, and HFA152a. In another embodiment, the at least one hydrofluoroalkane propellant consists of a double or triple combination of HFA134a, HFA227ea, and HFA152a.

In one embodiment, the at least one alcohol cosolvent is one alcohol cosolvent selected from the group consisting of ethanol and isopropyl alcohol. In another embodiment, the at least one alcohol cosolvent is a combination of ethanol and isopropyl alcohol.

In one embodiment of the pharmaceutical formulations of all of the aspects above, the compound of Formula (I), (Ia), (Ib), or (II), or an enantiomer, diastereomer, or pharmaceutically acceptable salt thereof is present at from about 0.5 to about 1 mg/mL, or about 1 mg/mL in the pharmaceutical formulation.

In one embodiment of the pharmaceutical formulations of all of the aspects above, the at least one PEGylated lipid is DSPE-PEG2000 present at from about 0.2 to about 3 mg/mL, from about 0.25 to about 0.75 mg/mL, or about 0.5 mg/mL in the pharmaceutical formulation.

In one embodiment of the pharmaceutical formulations of all of the aspects above, the at least one PEGylated lipid is DSG-PEG2000 present at from about 0.2 to about 0.5 mg/mL, or about 0.25 in the pharmaceutical formulation.

In one embodiment of the pharmaceutical formulations of all of the aspects above, the at least one PEGylated lipid is DPG-PEG2000 present at from about 0.2 to about 0.5 mg/mL, or at about 0.25 mg/mL in the pharmaceutical formulation.

In one embodiment of the pharmaceutical formulations of all of the aspects above, the at least one surfactant is PEG400 present at from about 0.75 to about 6 mg/mL, from about 1.5 to about 3 mg/mL, or about 3 mg/mL in the pharmaceutical formulation.

In one embodiment of the pharmaceutical formulations of all of the aspects above, the at least one surfactant is PEG1000 present at from about 0.75 to about 3 mg/mL, or about 3 mg/mL in the pharmaceutical formulation.

In one embodiment of the pharmaceutical formulations of all of the aspects above, the at least one surfactant is propylene glycol present at from about 0.75 to about 3 mg/mL, or about 1.5 mg/mL in the pharmaceutical formulation.

In one embodiment of the pharmaceutical formulations of all of the aspects above, the at least one alcohol cosolvent is ethanol present at from about 3% to about 10% (w/w), or from about 3% to about 5% (w/w), based on the total weight of the pharmaceutical formulation in the pharmaceutical formulation.

In one embodiment of the pharmaceutical formulations of all of the aspects above, the at least one alcohol cosolvent is isopropyl alcohol present at from about 5% to about 10% (w/w), or about 10% (w/w), based on the total weight of the pharmaceutical formulation in the pharmaceutical formulation.

In one embodiment of the pharmaceutical formulations of all of the aspects above, the at least one hydrofluoroalkane propellant is HFA134a. In another embodiment, the at least one hydrofluoroalkane propellant is HFA227ea. In yet another embodiment, the at least one hydrofluoroalkane propellant is HFA152a.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear dodecyl, linear tridecyl, linear tetradecyl, linear pentadecyl, linear hexadecyl, linear heptadecyl or linear octadecyl. In another embodiment, R₂ in the compound of Formula (I), (Ia), (Ib), or (II), or pharmaceutically acceptable salt thereof is linear dodecyl, linear tridecyl, linear tetradecyl, linear pentadecyl, linear hexadecyl, linear heptadecyl or linear octadecyl. In another embodiment, R₂ in the compound of Formula (I), (Ia), (Ib), or (II) is linear dodecyl, linear tridecyl, linear tetradecyl, linear pentadecyl, linear hexadecyl, linear heptadecyl or linear octadecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear dodecyl. In another embodiment, R₂ in the compound of Formula (I), (Ia), (Ib), or (II), or pharmaceutically acceptable salt thereof is linear dodecyl. In another embodiment, R₂ in the compound of Formula (I), (Ia), (Ib), or (II) is linear dodecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear tridecyl. In another embodiment, R₂ in the compound of Formula (I), (Ia), (Ib), or (II), or pharmaceutically acceptable salt thereof is linear tridecyl. In another embodiment, R₂ in the compound of Formula (I), (Ia), (Ib), or (II) is linear tridecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear tetradecyl. In another embodiment, R₂ in the compound of Formula (I), (Ia), (Ib), or (II), or pharmaceutically acceptable salt thereof is linear tetradecyl. In another embodiment, R₂ in the compound of Formula (I), (Ia), (Ib), or (II) is linear tetradecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear pentadecyl. In another embodiment, R₂ in the compound of Formula (I), (Ia), (Ib), or (II), or pharmaceutically acceptable salt thereof is linear pentadecyl. In another embodiment, R₂ in the compound of Formula (I), (Ia), (Ib), or (II) is linear pentadecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear hexadecyl. In another embodiment, R₂ in the compound of Formula (I), (Ia), (Ib), or (II), or pharmaceutically acceptable salt thereof is linear hexadecyl. In another embodiment, R₂ in the compound of Formula (I), (Ia), (Ib), or (II) is linear hexadecyl. In another embodiment, the compound of Formula (I), (Ia), or (II), or an enantiomer, diastereomer, or pharmaceutically acceptable salt thereof is a compound of Formula (III):

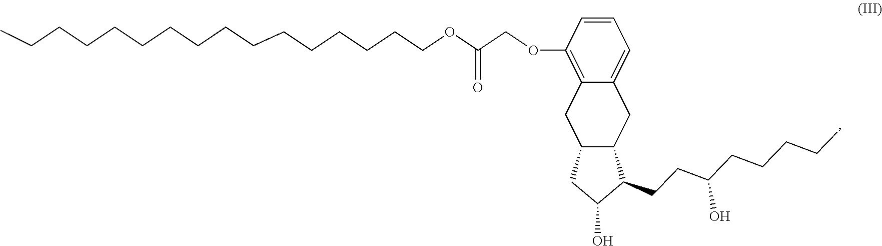

or an enantiomer, diastereomer, or pharmaceutically acceptable salt thereof. In one embodiment, the compound of Formula (I), (Ia), or (II), or pharmaceutically acceptable salt thereof is a compound of Formula (III) or a pharmaceutically acceptable salt thereof. In a further embodiment, the compound of Formula (I), (Ia), or (II), or pharmaceutically acceptable salt thereof is a compound of Formula (III).

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear heptadecyl. In another embodiment, R₂ in the compound of Formula (I), (Ia), (Ib), or (II), or pharmaceutically acceptable salt thereof is linear heptadecyl. In another embodiment, R₂ in the compound of Formula (I), (Ia), (Ib), or (II) is linear heptadecyl.

In one embodiment of the pharmaceutical formulations of all of the aspects above, R₂ in the compound of Formula (I), (Ia), (Ib), or (II), or an enantiomer, diastereomer, or pharmaceutically acceptable salt thereof is linear octadecyl. In another embodiment, R₂ in the compound of Formula (I), (Ia), (Ib), or (II), or pharmaceutically acceptable salt thereof is linear octadecyl. In another embodiment, R₂ in the compound of Formula (I), (Ia), (Ib), or (II) is linear octadecyl.

In one embodiment of the pharmaceutical formulations of all of the aspects above, (b) is polyoxyethylene (20) cetylether. In another embodiment of the pharmaceutical formulations of all of the aspects above, (b) is at least one polyethylene glycol-lipid (PEGylated lipid).

In one embodiment, the pharmaceutical formulation comprises 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a.

In another embodiment, the pharmaceutical formulation comprises 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In another embodiment, the pharmaceutical formulation comprises 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

In one embodiment, the pharmaceutical formulation consists of 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In another embodiment, the pharmaceutical formulation consists of 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In another embodiment, the pharmaceutical formulation consists of 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

In one embodiment of the pharmaceutical formulations of all of the aspects above, the pharmaceutical formulation is in the form of an aerosol. In some embodiments, the aerosol has a mass median aerodynamic diameter (MMAD) of from about 1 to about 3 μm, from about 1 to about 2 μm, or about 1.5 μm, as measured by Next Generation Impactor (NGI). In some embodiments, the aerosol has a throat deposition of from about 5% to about 40%, from about 10% to about 30%, from about 10% to about 25%, from about 15% to about 25%, or from about 15% to about 20%, as measured by NGI. In some embodiments, the aerosol has a fine particle fraction (FPF) of from about 50% to about 95%, from about 60% to about 85%, from about 70% to about 85%, from about 75% to about 85%, or from about 70% to about 80%, as measured by NGI.

In still another aspect, the present application provides a canister including a metering valve and the pharmaceutical formulation provided herein.

In still another aspect, the present application provides a metered dose inhaler (MDI) including the canister provided herein fitted into a suitable channeling device.

In still another aspect, the present application provides a method for treating pulmonary hypertension in a patient in need thereof. The method includes administering an effective amount of the pharmaceutical formulation provided herein to the lungs of the patient by inhalation via a metered dose inhaler.

In one embodiment, the pulmonary hypertension is pulmonary arterial hypertension.

In one embodiment, the pulmonary arterial hypertension is class I pulmonary arterial hypertension, as characterized by the New York Heart Association (NYHA). In another embodiment, the pulmonary arterial hypertension is class II pulmonary arterial hypertension, as characterized by the NYHA. In yet another embodiment, the pulmonary arterial hypertension is class III pulmonary arterial hypertension, as characterized by the NYHA. In yet another embodiment, the pulmonary arterial hypertension is class IV pulmonary arterial hypertension, as characterized by the NYHA.

In one embodiment, the pulmonary hypertension is group 1 pulmonary hypertension, as characterized by the World Health Organization (WHO). In another embodiment, the pulmonary hypertension is group 2 pulmonary hypertension, as characterized by the WHO. In yet another embodiment, the pulmonary hypertension is group 3 pulmonary hypertension, as characterized by the WHO. In yet another embodiment, the pulmonary hypertension is group 4 pulmonary hypertension, as characterized by the WHO. In yet another embodiment, the pulmonary hypertension is group 5 pulmonary hypertension, as characterized by the WHO.

In still another aspect, the present application provides a method for treating portopulmonary hypertension or pulmonary fibrosis in a patient in need thereof. The method includes administering an effective amount of the pharmaceutical formulation provided herein to the lungs of the patient by inhalation via a metered dose inhaler.

In still another aspect, the present application provides a system for treating pulmonary hypertension, portopulmonary hypertension, or pulmonary fibrosis. The system includes a pharmaceutical formulation provided herein, and an MDI provided herein.

### BRIEF DESCRIPTION OF THE FIGURES

FIG. 1 is a graph showing comparison of lung C16TR equivalents pharmacokinetics among nebulized INS1009 (1.0 mM), C16TR-dry powder (including C16TR, DSPE-PEG2000, mannitol, and leucine in a weight ratio of 1.5:0.75:70:30), and C16TR-MDI-W (high dose).

FIG. 2 is a graph showing comparison of plasma TRE pharmacokinetics among nebulized INS1009 (1.0 mM), C16TR-dry powder (including C16TR, DSPE-PEG2000, mannitol, and leucine in a weight ratio of 1.5:0.75:70:30), and C16TR-MDI-W (high dose).

FIG. 3 is a schematic showing transesterification of C16TR to 2C3TR in the presence of isopropyl alcohol (IPA).

FIG. 4A is a graph showing C16TR distribution with the C16TR-MDI-W formulation based on emitted dose in relation to actuator configuration.

FIG. 4B is a graph showing average mass deposition of treprostinil palmitil (C16TR) in different NGI Stages for the C16TR-MDI-W formulation with nozzle diameters of 0.12, 0.2, and 0.3 mm.

FIG. 5 is a graphical presentation of emitted dose vs number of actuations for valve up and valve down canisters containing the C16TR-MDI-W formulation.

FIG. 6A is a graph showing APSD % C16TR distribution of the C16TR-MDI-W formulation from dose through use studies—valve-up.

FIG. 6B is a graph showing APSD performance of the C16TR-MDI-W formulation from dose through use studies—valve-up. In each data set, four bars from left to right represent the beginning, middle, end, and mean values, respectively.

FIG. 7A is a graph showing APSD % C16TR distribution of the C16TR-MDI-W formulation from dose through use studies—valve-down.

FIG. 7B is a graph showing APSD performance of the C16TR-MDI-W formulation from dose through use studies-valve-down. In each data set, four bars from left to right represent the beginning, middle, end, and mean values, respectively.

FIG. 8 is a graph showing APSD comparison of MDI-X with MDI-W, with the left bar representing MDI-X and the right bar representing MDI-W in each data set in each NGI stage.

FIG. 9 is a graph showing comparison of lung C16TR equivalents pharmacokinetics among nebulized INS1009 (1.0 mM), C16TR-MDI-W (high dose), and C16TR-MI-W (low dose).

FIG. 10 is a graph showing comparison of plasma TRE pharmacokinetics among nebulized INS1009 (1.0 mM), C16TR-MDI-W (high dose), and C16TR-MI-W (low dose).

FIG. 11 is a graph showing comparison of lung C16TR equivalents pharmacokinetics between nebulized INS1009 (1.0 mM) and C16TR-MI-X.

FIG. 12 is a graph showing comparison of plasma TRE pharmacokinetics between nebulized INS1009 (1.0 mM) and C16TR-MDI-X.

FIG. 13 is a graph showing ΔRVPP response to hypoxic challenge in rats exposed to C16TR-MDI-W at a delivered dose of 115 μg/kg. Values are the mean±SEM (n=3). ΔRVPP: increase in right ventricular pulse pressure. Day −1 represents baseline values before drug. Day 0 represents post drug exposure. “*” denotes P<0.05, as compared to baseline on Day −1.

FIG. 14 is a graph showing ΔRVPP response to hypoxic challenge in rats exposed to C16TR-MDI-X at a delivered dose of 111 μg/kg. Values are the mean±SEM (n=3). ΔRVPP: increase in right ventricular pulse pressure. Day −1 represents baseline values before drug. Day 0 represents post drug exposure. “*” denotes P<0.05, as compared to baseline on Day −1.

FIG. 15 is a plot of cough counts vs. TRE delivered dose for MDI-TRE studies.

FIG. 16 is a zoomed-in plot of cough counts vs. TRE delivered dose for MDI-TRE studies.

FIG. 17 is a plot of cough counts vs. C16TR delivered dose in guinea pigs after inhalation of C16TR-MDI-L or C16TR-MDI-W.

FIG. 18 is a plot of cough counts vs. lung C16TR equivalents in guinea pigs after inhalation of C16TR-MDI-L or C16TR-MDI-W.

FIG. 19 is a plot of cough counts vs. C16TR delivered dose in guinea pigs after inhalation of C16TR-MI-L, C16TR-MDI-W, C16TR-DP1 (including C16TR, DSPE-PEG2000, mannitol, and leucine in a weight ratio of 1.5:0.75:70:30) or C16TR-DP2 (including C16TR, DSPE-PEG2000, trehalose, and leucine in a weight ratio of 1.5:0.75:70:30).

FIG. 20 is a plot of cough counts vs. lung C16TR equivalents in guinea pigs after inhalation of C16TR-MDI-L, C16TR-MDI-W, C16TR-DP1 (including C16TR, DSPE-PEG2000, mannitol, and leucine in a weight ratio of 1.5:0.75:70:30) or C16TR-DP2 (including C16TR, DSPE-PEG2000, trehalose, and leucine in a weight ratio of 1.5:0.75:70:30).

### DETAILED DESCRIPTION OF THE INVENTION

The term “alkyl” as used herein refers to both a linear alkyl, wherein alkyl chain length is indicated by a range of numbers, and a branched alkyl, wherein a branching point in the chain exists, and the total number of carbons in the chain is indicated by a range of numbers. In exemplary embodiments, “alkyl” refers to an alkyl chain as defined above containing 5, 6, 7, 8, 9, 10, 11, 12, 13, 14, 15, 16 carbons (i.e., C₅-C₁₆ alkyl). In one embodiment, the treprostinil alkyl ester of the MDI formulation provided herein is a linear alkyl having 14, 15, 16, 17 or 18 carbons. In a further embodiment, the linear alkyl is linear hexadecyl.

The term “pharmaceutically acceptable salt” refers to salts prepared from pharmaceutically acceptable non-toxic bases or acids including inorganic or organic bases and inorganic or organic acids. The nature of the salt is not critical, provided that it is pharmaceutically acceptable. Suitable pharmaceutically acceptable acid addition salts may be prepared from an inorganic acid or from an organic acid. Exemplary pharmaceutical salts are disclosed in Stahl, P. H., Wermuth, C. G., Eds. Handbook of Pharmaceutical Salts: Properties, Selection and Use; Verlag Helvetica Chimica Acta/Wiley-VCH: Zurich, 2002, the contents of which are hereby incorporated by reference in their entirety. Specific non-limiting examples of inorganic acids are hydrochloric, hydrobromic, hydroiodic, nitric, carbonic, sulfuric and phosphoric acid. Appropriate organic acids include, without limitation, aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric or galacturonic acid. Suitable pharmaceutically acceptable salts of free acid-containing compounds disclosed herein include, without limitation, metallic salts and organic salts. Exemplary metallic salts include, but are not limited to, appropriate alkali metal (group Ia) salts, alkaline earth metal (group IIa) salts, and other physiological acceptable metals. Such salts can be made from aluminum, calcium, lithium, magnesium, potassium, sodium and zinc.

Exemplary organic salts can be made from primary amines, secondary amines, tertiary amines and quaternary ammonium salts, for example, tromethamine, diethylamine, tetra-N-methylammonium, N,N′-dibenzylethylenediamine, chloroprocaine, choline, diethanolamine, ethylenediamine, meglumine (N-methylglucamine) and procaine.

Throughout the present specification, numerical ranges are provided for certain quantities. It is to be understood that these ranges comprise all subranges therein. Thus, the range “50-80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “treating” can include (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In another embodiment, “treating” refers to relieving the condition (for example, by causing regression of the state, disorder or condition or at least one of its clinical or subclinical symptoms). The benefit to a subject to be treated is either statistically significant as compared to the state or condition of the same subject before the treatment, or as compared to the state or condition of an untreated control subject, or the benefit is at least perceptible to the subject or to the physician.

“Effective amount” means an amount of a pharmaceutical formulation of the present disclosure that is sufficient to result in the desired therapeutic response. An effective amount of a pharmaceutical formulation can be administered in a single dose or in multiple doses.

Disclosed herein are pharmaceutical formulations suitable for administration by inhalation via a metered dose inhaler (MDI). The present application also discloses methods for preparation of the pharmaceutical formulations and their use in therapy. In one aspect, the present disclosure provides pharmaceutical formulations of treprostinil prodrugs that may be used for treating, for example, pulmonary hypertension (e.g., pulmonary arterial hypertension), portopulmonary hypertension, or pulmonary fibrosis in a patient in need thereof. In one embodiment, the pharmaceutical formulation includes (a) a compound of Formula (I):

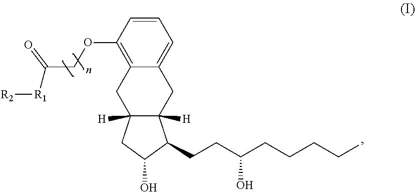

or an enantiomer, diastereomer, or pharmaceutically acceptable salt thereof, wherein R₁ is NH, O or S; R₂ is a linear or branched C₅-C₁₈ alkyl, a linear C₂-C₁₈ alkenyl or a branched C₃-C₁₈ alkenyl, aryl, aryl-C₁-C₁₈ alkyl, an amino acid or a peptide; and n is an integer from 0 to 5; (b) polyoxyethylene (20) cetylether or at least one polyethylene glycol-lipid (PEGylated lipid); (c) a surfactant selected from polyethylene glycol (PEG), propylene glycol, or a combination thereof, (d) at least one hydrofluoroalkane propellant, and (e) at least one alcohol cosolvent. In one embodiment, (a) is a compound of Formula (I) or a pharmaceutically acceptable salt thereof. In a further embodiment, (a) is a compound of Formula (I). The compound of Formula (I) and pharmaceutically acceptable salts thereof are treprostinil prodrugs as disclosed in International Application Publication WO 2015/061720, incorporated herein by reference in its entirety.

In one embodiment, the compound of Formula (I), or an enantiomer, diastereomer, or pharmaceutically acceptable salt thereof, is a compound of Formula (Ia):

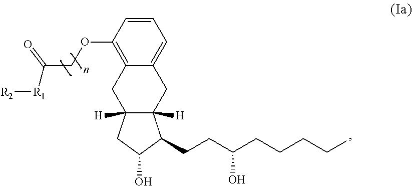

or an enantiomer, diastereomer, or pharmaceutically acceptable salt thereof, wherein R₁ is O; R₂ is dodecyl, tridecyl, tetradecyl, pentadecyl, hexadecyl, heptadecyl or octadecyl; and n is an integer from 0 to 5. In one embodiment, the compound of Formula (I) or pharmaceutically acceptable salt thereof is a compound of Formula (Ia) or a pharmaceutically acceptable salt thereof, i.e., (a) is a compound of Formula (Ia) or a pharmaceutically acceptable salt thereof in the pharmaceutical formulation. In another embodiment, the compound of Formula (I) or pharmaceutically acceptable salt thereof is a compound of Formula (Ia), i.e., (a) is a compound of Formula (Ia) in the pharmaceutical formulation. In one embodiment, n is 0 or 1. In another embodiment, n is 0. In another embodiment, R₁ is O; R₂ is dodecyl, tridecyl, tetradecyl, pentadecyl, hexadecyl, heptadecyl or octadecyl, and n is 1, whereby the compound of Formula (Ia), or an enantiomer, diastereomer, or pharmaceutically acceptable salt thereof, can be represented by a compound of Formula (II):

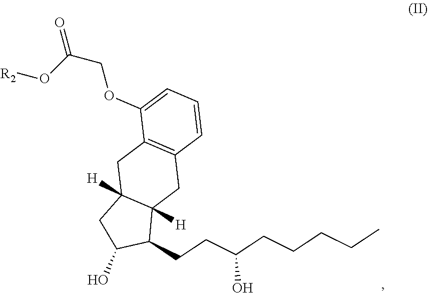

or an enantiomer, diastereomer, or pharmaceutically acceptable salt thereof. In one embodiment, the compound of Formula (Ia) or pharmaceutically acceptable salt thereof is a compound of Formula (II) or a pharmaceutically acceptable salt thereof in the pharmaceutical formulation. In another embodiment, the compound of Formula (Ia) or pharmaceutically acceptable salt thereof is a compound of Formula (II).

In one embodiment of the compound of Formula (I), (Ia), or (II), or an enantiomer, diastereomer, or pharmaceutically acceptable salt thereof, R₂ is a linear C₁₄-C₁₈ alkyl. In a further embodiment, R₂ in the compound of Formula (I), (Ia), or (II), or pharmaceutically acceptable salt thereof is a linear C₁₄-C₁₈ alkyl. In a further embodiment, R₂ in the compound of Formula (I), (Ia), or (II) is a linear C₁₄-C₁₈ alkyl.

In another embodiment of the compound of Formula (I), (Ia), or (II), or an enantiomer, diastereomer, or pharmaceutically acceptable salt thereof, R₂ is dodecyl. In a further embodiment, R₂ is linear dodecyl. In one embodiment, R₂ in the compound of Formula (I), (Ia), or (II), or pharmaceutically acceptable salt thereof is linear dodecyl. In another embodiment, R₂ in the compound of Formula (I), (Ia), or (II) is linear dodecyl.

In another embodiment of the compound of Formula (I), (Ia), or (II), or an enantiomer, diastereomer, or pharmaceutically acceptable salt thereof, R₂ is tridecyl. In a further embodiment, R₂ is linear tridecyl. In one embodiment, R₂ in the compound of Formula (I), (Ia), or (II), or pharmaceutically acceptable salt thereof is linear tridecyl. In another embodiment, R₂ in the compound of Formula (I), (Ia), or (II) is linear tridecyl.

In another embodiment of the compound of Formula (I), (Ia), or (II), or an enantiomer, diastereomer, or pharmaceutically acceptable salt thereof, R₂ is tetradecyl. In a further embodiment, R₂ is linear tetradecyl. In one embodiment, R₂ in the compound of Formula (I), (Ia), or (II), or pharmaceutically acceptable salt thereof is linear tetradecyl. In another embodiment, R₂ in the compound of Formula (I), (Ia), or (II) is linear tetradecyl.

In another embodiment of the compound of Formula (I), (Ia), or (II), or an enantiomer, diastereomer, or pharmaceutically acceptable salt thereof, R₂ is pentadecyl. In a further embodiment, R₂ is linear pentadecyl. In one embodiment, R₂ in the compound of Formula (I), (Ia), or (II), or pharmaceutically acceptable salt thereof is linear pentadecyl. In another embodiment, R₂ in the compound of Formula (I), (Ia), or (II) is linear pentadecyl.

In another embodiment of the compound of Formula (I), (Ia), or (II), or an enantiomer, diastereomer, or pharmaceutically acceptable salt thereof, R₂ is heptadecyl. In a further embodiment, R₂ is linear heptadecyl. In one embodiment, R₂ in the compound of Formula (I), (Ia), or (II), or pharmaceutically acceptable salt thereof is linear heptadecyl. In another embodiment, R₂ in the compound of Formula (I), (Ia), or (II) is linear heptadecyl.

In another embodiment of the compound of Formula (I), (Ia), or (II), or an enantiomer, diastereomer, or pharmaceutically acceptable salt thereof, R₂ is octadecyl. In a further embodiment, R₂ is linear octadecyl. In one embodiment, R₂ in the compound of Formula (I), (Ia), or (II), or pharmaceutically acceptable salt thereof is linear octadecyl. In another embodiment, R₂ in the compound of Formula (I), (Ia), or (II) is linear octadecyl.

In another embodiment of the compound of Formula (I) or (Ia), or an enantiomer, diastereomer, or pharmaceutically acceptable salt thereof, R₂ is hexadecyl. In a further embodiment, R₂ is linear hexadecyl. In one embodiment, R₂ in the compound of Formula (I) or (Ia), or pharmaceutically acceptable salt thereof is linear hexadecyl. In another embodiment, R₂ in the compound of Formula (I) or (Ia) is linear hexadecyl.

In another embodiment of the compound of Formula (II), or an enantiomer, diastereomer, or pharmaceutically acceptable salt thereof, R₂ is hexadecyl. In a further embodiment, the hexadecyl is linear hexadecyl, whereby the compound of Formula (II), or an enantiomer, diastereomer, or pharmaceutically acceptable salt thereof, is a compound of Formula (III):

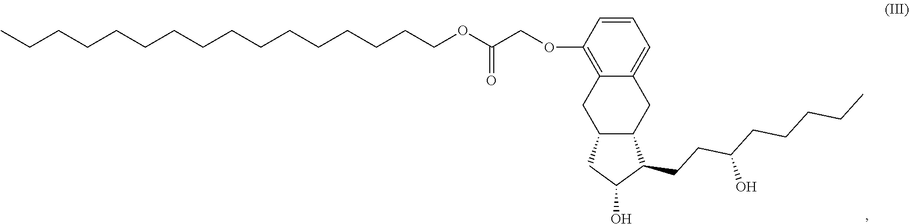 [SMILES: CCCCCCCCCCCCCCCCOC(=O)COc1cccc2c1C[C@H]1C[C@@H](O)[C@H](CC[C@@H](O)CCCCC)[C@H]1C2]

or an enantiomer, diastereomer, or pharmaceutically acceptable salt thereof. In one embodiment, the compound of Formula (II) or pharmaceutically acceptable salt thereof is a compound of Formula (III) or a pharmaceutically acceptable salt thereof. In a further embodiment, the compound of Formula (II) or pharmaceutically acceptable salt thereof is a compound of Formula (III).

The compound of Formula (III) is also referred to herein as C16TR or treprostinil palmitil. In one embodiment, the compound of Formula (I), (Ia), or (II), or an enantiomer, diastereomer, or pharmaceutically acceptable salt thereof is a compound of Formula (III), or an enantiomer, diastereomer, or pharmaceutically acceptable salt thereof, i.e., (a) is a compound of Formula (III), or an enantiomer, diastereomer, or pharmaceutically acceptable salt thereof in the pharmaceutical formulation. In another embodiment, the compound of Formula (I), (Ia), or (II), or pharmaceutically acceptable salt thereof is a compound of Formula (III) or a pharmaceutically acceptable salt thereof, i.e., (a) is a compound of Formula (III) or a pharmaceutically acceptable salt thereof in the pharmaceutical formulation. In another embodiment, the compound of Formula (I), (Ia), or (II), or pharmaceutically acceptable salt thereof is a compound of Formula (III), i.e., (a) is a compound of Formula (III) in the pharmaceutical formulation.

In another embodiment, the compound of Formula (I), or an enantiomer, diastereomer, or pharmaceutically acceptable salt thereof, is a compound of Formula (Ib):

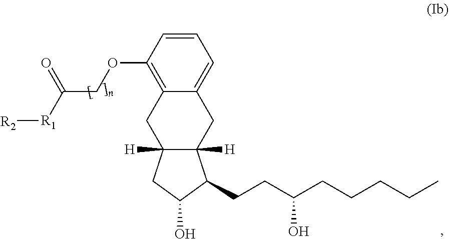

or an enantiomer, diastereomer, or pharmaceutically acceptable salt thereof, wherein R₁ is NH or O, R₂ is a linear C₅-C₁₅ alkyl, and n is 0. In one embodiment, the compound of Formula (I) or pharmaceutically acceptable salt thereof is a compound of Formula (Ib) or a pharmaceutically acceptable salt thereof, i.e., (a) is a compound of Formula (Ib) or a pharmaceutically acceptable salt thereof in the pharmaceutical formulation. In another embodiment, the compound of Formula (I) or pharmaceutically acceptable salt thereof is a compound of Formula (Ib), i.e., (a) is a compound of Formula (Ib) in the pharmaceutical formulation. In some embodiments, R₂ is linear heptyl, linear octyl, linear nonyl, linear decyl, linear undecyl, linear dodecyl, linear tridecyl, linear tetradecyl, linear pentadecyl, linear hexadecyl, linear heptadecyl or linear octadectyl. In one embodiment, R₁ is NH. In another embodiment, R₁ is O.

In yet another embodiment of the compound of Formula (I), R₁ is O, R₂ is a linear C₁₄-C₁₈ alkyl, and n is 1. In a further embodiment, R₂ is a linear hexadecyl.

In some embodiments, the compound of Formula (I), (Ia), (Ib), (II), or (III), or an enantiomer, diastereomer, or pharmaceutically acceptable salt thereof, is present at a concentration of from about 0.5 to about 3 mg/mL, from about 0.5 to about 1 mg/mL, about 0.5 mg/mL, or about 1 mg/mL in the pharmaceutical formulation. In one embodiment, the compound of Formula (I), (Ia), (Ib), (II), or (III), or an enantiomer, diastereomer, or pharmaceutically acceptable salt thereof is present at a concentration of from about 0.5 to about 3 mg/mL in the pharmaceutical formulation.

In another embodiment, the compound of Formula (I), (Ia), (Ib), (II), or (III), or an enantiomer, diastereomer, or pharmaceutically acceptable salt thereof is present at a concentration of from about 0.5 to about 1 mg/mL in the pharmaceutical formulation. In still another embodiment, the compound of Formula (I), (Ia), (Ib), (II), or (III), or an enantiomer, diastereomer, or pharmaceutically acceptable salt thereof is present at a concentration of about 0.5 mg/mL in the pharmaceutical formulation. In still another embodiment, the compound of Formula (I), (Ia), (Ib), (II), or (III), or an enantiomer, diastereomer, or pharmaceutically acceptable salt thereof is present at a concentration of about 1 mg/mL in the pharmaceutical formulation. In some embodiments, the compound of Formula (I), (Ia), (Ib), (II), or (III), or pharmaceutically acceptable salt thereof, is present at each of the above-mentioned concentrations or concentration ranges in the pharmaceutical formulation. In some embodiments, the compound of Formula (I), (Ia), (Ib), (II), or (III) is present at each of the above-mentioned concentrations or concentration ranges in the pharmaceutical formulation.

In one embodiment, the pharmaceutical formulation includes polyoxyethylene (20) cetyl ether (e.g., sold under the trade name Brij® 58). Polyoxyethylene (20) cetylether may be present at a concentration of from about 0.25 to about 0.75 mg/mL or about 0.5 mg/mL in the pharmaceutical formulation. In one embodiment, polyoxyethylene (20) cetylether is present at a concentration of about 0.5 mg/mL in the pharmaceutical formulation.

PEG refers to polyethylene glycol, also known as polyethylene oxide (PEO) or polyoxyethylene (POE). PEGs are prepared by polymerization of ethylene oxide and are commercially available over a wide range of molecular weights from 100 g/mol to 10,000,000 g/mol. The numbers included in the names of PEGs indicate their average molecular weights. For example, PEG400 denotes a PEG having an average molecular weight of approximately 400 g/mol, and PEG1000 denotes a PEG having an average molecular weight of approximately 1000 g/mol. A PEG may be covalently coupled to a lipid to form a PEGylated lipid.

In one embodiment, the pharmaceutical formulation of the present disclosure includes one or more PEGylated lipids, and does not include polyoxyethylene (20) cetyl ether. In one embodiment, the pharmaceutical formulation includes only one PEGylated lipid. In another embodiment, the pharmaceutical formulation includes more than one PEGylated lipid. In some embodiments, the PEG of the PEGylated lipids in the pharmaceutical formulation have an average molecular weight ranging from 500 to 10,000 g/mol, from 1000 to 5000 g/mol, or at approximately 2000 g/mol. In some embodiments, the one or more PEGylated lipids in the pharmaceutical formulation are PEGylated phospholipids, such as PEGylated phosphatidylcholines (e.g., dioleoyl phosphatidylcholine, dilauroyl phosphatidylcholine, dimyristoyl phosphatidylcholine, dipalmitoyl phosphatidylcholine, and distearoyl phosphatidylcholine), PEGylated phosphatidylglycerols (e.g., dioleoyl phosphatidylglycerol, dilauroyl phosphatidylglycerol, dimyristoyl phosphatidylglycerol, dipalmitoyl phosphatidylglycerol, and distearoyl phosphatidylglycerol), PEGylated phosphatidylethanolamines (e.g., dioleoyl phosphatidylethanolamine, dilauroyl phosphatidylethanolamine, dimyristoyl phosphatidylethanolamine, dipalmitoyl phosphatidylethanolamine, and distearoyl phosphatidylethanolamine), PEGylated phosphatidylserines, PEGylated phosphatidylinositols, and PEGylated phosphatidic acid. In other embodiments, the one or more PEGylated lipids in the pharmaceutical formulation include, but are not limited to, DSPE (distearoylphosphatidylethanolamine)-PEG2000, DSG (disteraroylglycerol)-PEG2000, DPG (diphosphatidylglycerol)-PEG2000, DMPE (dimyristoyl phosphatidylethanolamrine)-PEG2000, DMG (dimyristoyl glycerol)-PEG2000, cholesterylated PEG2000, STR (Stearyl)-PEG2000, or a combination thereof. In one embodiment, the one or more PEGylated lipids in the pharmaceutical formulation are selected from DSPE-PEG2000, DSG-PEG2000, DPG-PEG2000, and a combination thereof. DSPE-PEG2000, DSG-PEG2000, or DPG-PEG2000 may include a branched or unbranched PEG molecule with an average PEG molecular weight of 2000 g/mol. In some embodiments, the one or more PEGylated lipids, e.g., DSPE-PEG2000, DSG-PEG2000, DPG-PEG2000, or their double or triple combinations, are present at a concentration of from about 0.2 to about 3 mg/mL, from about 0.25 to about 0.75 mg/mL, from about 0.2 to about 0.5 mg/mL; about 0.5 mg/mL, or about 0.25 mg/mL in the pharmaceutical formulation.

In one embodiment, the pharmaceutical formulation includes only one PEGylated lipid selected from DSPE-PEG2000, DSG-PEG2000, and DPG-PEG2000. In another embodiment, the pharmaceutical formulation includes double or triple combinations of the above-mentioned PEGylated lipids, i.e., DSPE-PEG2000+DSG-PEG2000, DSPE-PEG2000+DPG-PEG2000, DSG-PEG2000+DPG-PEG2000, or DSPE-PEG2000+DSG-PEG2000+DPG-PEG2000.

In one embodiment, the at least one PEGylated lipid is DSPE-PEG2000 present at from about 0.2 to about 3 mg/mL. In another embodiment, the at least one PEGylated lipid is DSPE-PEG2000 present at from about 0.25 to about 0.75 mg/mL. In another embodiment, the at least one PEGylated lipid is DSPE-PEG2000 present at about 0.5 mg/mL.

In one embodiment, the at least one PEGylated lipid is DSG-PEG2000 present at from about 0.2 to about 0.5 mg/mL. In another embodiment, the at least one PEGylated lipid is DSG-PEG2000 present at about 0.25 mg/mL.

In one embodiment, the at least one PEGylated lipid in the pharmaceutical formulation is DPG-PEG2000 present at from about 0.2 to about 0.5 mg/mL. In another embodiment, the at least one PEGylated lipid is DPG-PEG2000 present at about 0.25 mg/mL.

The pharmaceutical formulation provided herein includes one or more surfactants. Exemplary surfactants include, but are not limited to, propylene glycol and polyethyleneglycol (PEG) with an average molecular weight ranging from 100 to 1500, 200 to 1000, or 300 to 500 g/mol, e.g., PEG200, PEG400, or PEG1000. In the present application, where PEG is used as the surfactant, the PEG is a separate component from the PEGylated lipid described above. In some embodiments, the pharmaceutical formulation includes a surfactant selected from PEG400, PEG1000, propylene glycol, or a combination thereof. In other embodiments, the pharmaceutical formulation includes only one surfactant selected from the group consisting of PEG400, PEG1000, and propylene glycol. In still other embodiments, the pharmaceutical formulation includes double or triple combinations of the above-mentioned surfactants, i.e., PEG400+PEG1000, PEG400+propylene glycol, PEG1000+propylene glycol, or PEG400+PEG1000+propylene glycol.

In some embodiments, the one or more surfactants, e.g., PEG400, PEG1000, propylene glycol, or their double or triple combinations, are present at a concentration of from about 0.75 to about 6 mg/mL; from about 0.75 to about 3 mg/mL, from about 1.5 to about 3 mg/mL; about 3 mg/mL, or about 1.5 mg/mL in the pharmaceutical formulation.

In some embodiments, incorporation of PEG400 in one of the formulations described herein yields a MMAD, e.g., from about 1 to about 5 μm, or from about 1.5 to about 3 μm, when measured by NGI, regardless of other excipients used in the formulation, and a favorable pharmacokinetics performance of the formulation when delivered to the lungs by a metered dose inhaler (MDI), e.g., less C16TR remaining in lung tissues 24 h after dosing as shown in the examples. In one embodiment, the surfactant in the pharmaceutical formulation is PEG400 present at from about 0.75 to about 6 mg/mL in the pharmaceutical formulation. In another embodiment, the surfactant is PEG400 present at from about 1.5 to about 3 mg/mL in the pharmaceutical formulation. In still another embodiment, the surfactant is PEG400 present at about 3 mg/mL in the pharmaceutical formulation.

In one embodiment, the surfactant in the pharmaceutical formulation is PEG1000 present at from about 0.75 to about 3 mg/mL in the pharmaceutical formulation. In another embodiment, the surfactant is PEG1000 present at about 1.5 mg/mL in the pharmaceutical formulation. In still another embodiment, the surfactant is PEG1000 present at about 3 mg/mL in the pharmaceutical formulation.

In one embodiment, the surfactant in the pharmaceutical formulation is propylene glycol present at from about 0.75 to about 3 mg/mL in the pharmaceutical formulation. In another embodiment, the surfactant is propylene glycol present at about 1.5 mg/mL in the pharmaceutical formulation. In still another embodiment, the surfactant is propylene glycol present at about 3 mg/mL in the pharmaceutical formulation.

The pharmaceutical formulation provided herein includes one or more alcohol cosolvents. In one embodiment, the pharmaceutical formulation includes only one alcohol cosolvent. In another embodiment, the pharmaceutical formulation includes more than one alcohol cosolvent, e.g., a mixture of multiple alcohol cosolvents. Exemplary alcohol cosolvents include ethanol and isopropyl alcohol. In one embodiment, the pharmaceutical formulation includes only one alcohol cosolvent selected from the group consisting of ethanol and isopropyl alcohol. In another embodiment, the pharmaceutical formulation includes a combination or mixture of ethanol and isopropyl alcohol as the alcohol cosolvent.

In some embodiments, the one or more alcohol cosolvents, e.g., ethanol (EtOH), isopropyl alcohol (IPA), or a mixture of ethanol and isopropyl alcohol, are present at a concentration of from about 3% to about 10% (w/w), from about 5% to about 10% (w/w), from about 3% to about 5% (w/w), about 3% (w/w), about 5% (w/w), about 7% (w/w), about 9% (w/w), or about 10% (w/w) based on the total weight of the pharmaceutical formulation.

EtOH is the industry standard alcohol co-solvent for MDI formulations. A high concentration of EtOH may lead to a decrease in aerosol performance and significantly more throat deposition. In one embodiment, the at least one alcohol cosolvent in the pharmaceutical formulation is ethanol present at from about 3% to about 10% (w/w) based on the total weight of the pharmaceutical formulation. In another embodiment, the at least one alcohol cosolvent in the pharmaceutical formulation is ethanol present at from about 3% to about 5% (w/w) based on the total weight of the pharmaceutical formulation.

Isopropyl alcohol (IPA) may be used as a co-solvent in embodiments described herein. In one embodiment, the at least one alcohol cosolvent in the pharmaceutical formulation is isopropyl alcohol present at from about 5% to about 10% (w/w) based on the total weight of the pharmaceutical formulation. In another embodiment, the at least one alcohol cosolvent in the pharmaceutical formulation is isopropyl alcohol present at about 10% (w/w) based on the total weight of the pharmaceutical formulation. In another embodiment, the at least one alcohol cosolvent in the pharmaceutical formulation is isopropyl alcohol present at about 7% (w/w) based on the total weight of the pharmaceutical formulation.

The pharmaceutical formulation of the present invention includes one or more hydrofluoroalkane propellants. In one embodiment, the pharmaceutical formulation includes only one hydrofluoroalkane propellant. In another embodiment, the pharmaceutical formulation includes more than one hydrofluoroalkane propellant, e.g., a mixture of multiple hydrofluoroalkane propellants. Exemplary hydrofluoroalkane propellants amenable for use herein include 1,1,1,2-tetrafluoroethane (HFA134a), 1,1,1,2,3,3,3-heptafluoropropane (HFA227ea), and 1,1-difluoroethane (HFA152a). In some embodiments, the pharmaceutical formulation includes only one hydrofluoroalkane propellant selected from the group consisting of HFA134a, HFA227ea, and HFA152a. In one embodiment, the pharmaceutical formulation includes only one hydrofluoroalkane propellant which is HFA134a. In another embodiment, the pharmaceutical formulation includes only one hydrofluoroalkane propellant which is HFA227ea. In still another embodiment, the pharmaceutical formulation includes only one hydrofluoroalkane propellant which is HFA152a. In still another embodiment, the pharmaceutical formulation includes a mixture of HFA134a and HFA227ea as the propellant. In still another embodiment, the pharmaceutical formulation includes a combination or mixture of HFA134a and HFA152a as the propellant. In still another embodiment, the pharmaceutical formulation includes a combination or mixture of HFA227ea and HFA152a as the propellant. In still another embodiment, the pharmaceutical formulation includes a combination or mixture of HFA134a, HFA227ea, and HFA152a as the propellant.

In one embodiment, the pharmaceutical formulation comprises 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation comprises 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation comprises 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

Mass median aerodynamic diameter (MMAD) is the value of aerodynamic diameter for which 50% of the mass in a given aerosol is associated with particles smaller than the median aerodynamic diameter (MAD), and 50% of the mass is associated with particles larger than the MAD. MMAD can be determined by impactor measurements, e.g., the Andersen Cascade Impactor (ACI) or the Next Generation Impactor (NGI). In some embodiments, the pharmaceutical formulation is in the form of an aerosol, which may be generated by, for example, actuation of a metered dose inhaler (MDI). The aerosol of the formulations described herein, in one embodiment, comprises particles with an MMAD of from about 1 to about 3 μm, from about 1 to about 2 μm, or about 1.5 μm, as measured by Next Generation Impactor (NGI).

Throat deposition is the amount of drug deposited on the throat stage of a cascade impactor and is expressed as a percentage. In some embodiments, the pharmaceutical formulation is in the form of an aerosol having a throat deposition of from about 5% to about 40%, from about 10% to about 30%, from about 10% to about 25%, from about 15% to about 25%, or from about 15% to about 20%, as measured by NGI. The aerosol may be generated by, for example, actuation of an MDI.

“Fine particle fraction” or “FPF” refers to the fraction of an aerosol having a particle size less than 5 μm in diameter, as measured by cascade impaction. FPF is usually expressed as a percentage. FPF has been demonstrated to correlate to the fraction of an aerosol that is deposited in the lungs of a patient. In some embodiments, the pharmaceutical formulation is in the form of an aerosol comprising particles with an FPF of from about 50% to about 95%, from about 60% to about 85%, from about 70% to about 85%, from about 75% to about 85%, or from about 70% to about 80%, as measured by NGI. The aerosol of the formulations described herein may be generated by, for example, actuation of an MDI.

In another aspect the present invention provides, a pharmaceutical formulation comprising or consisting of (a) a compound of Formula (II):

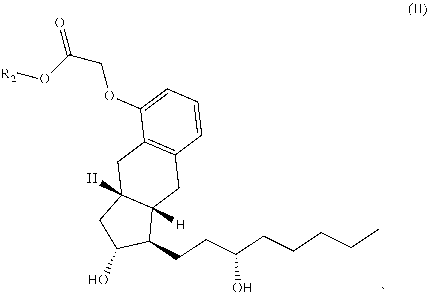

or an enantiomer, diastereomer, or pharmaceutically acceptable salt thereof at a concentration of from about 0.5 to about 3 mg/mL, from about 0.5 to about 1 mg/mL, about 0.5 mg/mL, or about 1 mg/mL,

wherein R₂ is dodecyl, tridecyl, tetradecyl, pentadecyl, hexadecyl, heptadecyl, or octadecyl,
(b) polyoxyethylene (20) cetylether at a concentration of from about 0.25 to about 0.75 mg/mL or about 0.5 mg/mL, or at least one PEGylated lipid selected from the group consisting of DSPE-PEG2000, DSG-PEG2000, and DPG-PEG2000 at a concentration of from about 0.2 to about 3 mg/mL, from about 0.25 to about 0.75 mg/mL, from about 0.2 to about 0.5 mg/mL, about 0.5 mg/mL, or about 0.25 mg/mL,
(c) a surfactant selected from PEG400, PEG1000, propylene glycol, or a combination thereof at a concentration of from about 0.75 to about 6 mg/mL, from about 0.75 to about 3 mg/mL, from about 1.5 to about 3 mg/mL, about 3 mg/mL, or about 1.5 mg/mL,
(d) at least one hydrofluoroalkane propellant selected from the group consisting of HFA134a, HFA227ea, and HFA152a, and
(e) at least one alcohol cosolvent selected from the group consisting of ethanol and isopropyl alcohol at a concentration of from about 3% to about 10% (w/w), from about 5% to about 10% (w/w), from about 30% to about 50% (w/w), about 30% (w/w), about 50% (w/w), about 7% (w/w), about 9% (w/w), or about 10% (w/w) based on the total weight of the pharmaceutical formulation.

In one embodiment of a formulation described herein, (a) is a compound of Formula (II) or a pharmaceutically acceptable salt thereof. In a further embodiment, (a) is a compound of Formula (II).

In one embodiment, the compound of Formula (II), or an enantiomer, diastereomer, or pharmaceutically acceptable salt thereof is present at a concentration of from about 0.5 to about 3 mg/mL. In another embodiment, the compound of Formula (II), or an enantiomer, diastereomer, or pharmaceutically acceptable salt thereof is present at a concentration of from about 0.5 to about 1 mg/mL. In another embodiment, the compound of Formula (II), or an enantiomer, diastereomer, or pharmaceutically acceptable salt thereof is present at a concentration of about 1 mg/mL. In some embodiments, the compound of Formula (II) or pharmaceutically acceptable salt thereof is present at each of the above-mentioned concentrations or concentration ranges in the pharmaceutical formulation. In some embodiments, the compound of Formula (II) is present at each of the above-mentioned concentrations or concentration ranges in the pharmaceutical formulation.

In one embodiment, R₂ is dodecyl in the compound of Formula (II), or an enantiomer, diastereomer, or pharmaceutically acceptable salt thereof. In a further embodiment, R₂ is linear dodecyl. In one embodiment, R₂ in the compound of Formula (II) or pharmaceutically acceptable salt thereof is linear dodecyl. In another embodiment, R₂ in the compound of Formula (II) is linear dodecyl.

In one embodiment, R₂ is tridecyl in the compound of Formula (II), or an enantiomer, diastereomer, or pharmaceutically acceptable salt thereof. In a further embodiment, R₂ is linear tridecyl. In one embodiment, R₂ in the compound of Formula (II) or pharmaceutically acceptable salt thereof is linear tridecyl. In another embodiment, R₂ in the compound of Formula (II) is linear tridecyl.

In one embodiment, R₂ is tetradecyl in the compound of Formula (II), or an enantiomer, diastereomer, or pharmaceutically acceptable salt thereof. In a further embodiment, R₂ is linear tetradecyl. In one embodiment, R₂ in the compound of Formula (II) or pharmaceutically acceptable salt thereof is linear tetradecyl. In another embodiment, R₂ in the compound of Formula (II) is linear tetradecyl.

In one embodiment, R₂ is pentadecyl in the compound of Formula (II), or an enantiomer, diastereomer, or pharmaceutically acceptable salt thereof. In a further embodiment, R₂ is linear pentadecyl. In one embodiment, R₂ in the compound of Formula (II) or pharmaceutically acceptable salt thereof is linear pentadecyl. In another embodiment, R₂ in the compound of Formula (II) is linear pentadecyl.

In one embodiment, R₂ is hexadecyl in the compound of Formula (II), or an enantiomer, diastereomer, or pharmaceutically acceptable salt thereof. In a further embodiment, the hexadecyl is linear hexadecyl, i.e., the compound of Formula (II), or an enantiomer, diastereomer, or pharmaceutically acceptable salt thereof is a compound of Formula (III), or an enantiomer, diastereomer, or pharmaceutically acceptable salt thereof. In one embodiment, the compound of Formula (II) or pharmaceutically acceptable salt thereof is a compound of Formula (III) or a pharmaceutically acceptable salt thereof. In another embodiment, the compound of Formula (II) or pharmaceutically acceptable salt thereof is a compound of Formula (III).

In another embodiment, R₂ is heptadecyl in the compound of Formula (II), or an enantiomer, diastereomer, or pharmaceutically acceptable salt thereof. In a further embodiment, R₂ is linear heptadecyl. In one embodiment, R₂ in the compound of Formula (II) or pharmaceutically acceptable salt thereof is linear heptadecyl. In another embodiment, R₂ in the compound of Formula (II) is linear heptadecyl.

In another embodiment, R₂ is octadecyl in the compound of Formula (II), or an enantiomer, diastereomer, or pharmaceutically acceptable salt thereof. In a further embodiment, R₂ is linear octadecyl. In one embodiment, R₂ in the compound of Formula (II) or pharmaceutically acceptable salt thereof is linear octadecyl. In another embodiment, R₂ in the compound of Formula (II) is linear octadecyl.

In one embodiment, (b) is polyoxyethylene (20) cetylether (sold under the trade name Brij® 58) at a concentration of from about 0.25 to about 0.75 mg/mL. In another embodiment, polyoxyethylene (20) cetylether is present at a concentration of about 0.5 mg/mL.

In one embodiment, (b) is at least one PEGylated lipid selected from the group consisting of DSPE-PEG2000, DSG-PEG2000, and DPG-PEG2000 at a concentration of from about 0.2 to about 3 mg/mL. In another embodiment, the at least one PEGylated lipid is present at a concentration of from about 0.2 to about 0.5 mg/mL. In another embodiment, the at least one PEGylated lipid is present at a concentration of from about 0.25 to about 0.75 mg/mL. In another embodiment, the at least one PEGylated lipid is present at a concentration of about 0.5 mg/mL. In another embodiment, the at least one PEGylated lipid is present at a concentration of about 0.25 mg/mL. In some embodiments, the at least one PEGylated lipid is one PEGylated lipid selected from the group consisting of DSPE-PEG2000, DSG-PEG2000, and DPG-PEG2000. In other embodiments, the at least one PEGylated lipid consists of a double or triple combination of DSPE-PEG2000, DSG-PEG2000, and DPG-PEG2000.

In one embodiment, the surfactant is present at a concentration of from about 0.75 to about 6 mg/mL. In another embodiment, the surfactant is present at a concentration of from about 1.5 to about 3 mg/mL. In another embodiment, the surfactant is present at a concentration of about 3 mg/mL. In another embodiment, the surfactant is present at a concentration of about 1.5 mg/mL. In some embodiments, the surfactant is one surfactant selected from the group consisting of PEG400, PEG1000, and propylene glycol. In other embodiments, the surfactant consists of a double or triple combination of PEG400, PEG1000, and propylene glycol.

In one embodiment, the at least one alcohol cosolvent is present at a concentration of from about 3% to about 10% (w/w) based on the total weight of the pharmaceutical formulation.

In another embodiment, the at least one alcohol cosolvent is present at a concentration of from about 5% to about 10% (w/w) based on the total weight of the pharmaceutical formulation. In another embodiment, the at least one alcohol cosolvent is present at a concentration of from about 3% to about 5% (w/w) based on the total weight of the pharmaceutical formulation. In another embodiment, the at least one alcohol cosolvent is present at a concentration of about 10% (w/w) based on the total weight of the pharmaceutical formulation. In another embodiment, the at least one alcohol cosolvent is present at a concentration of about 9% (w/w) based on the total weight of the pharmaceutical formulation. In another embodiment, the at least one alcohol cosolvent is present at a concentration of about 7% (w/w) based on the total weight of the pharmaceutical formulation. In another embodiment, the at least one alcohol cosolvent is present at a concentration of about 5% (w/w) based on the total weight of the pharmaceutical formulation. In another embodiment, the at least one alcohol cosolvent is present at a concentration of about 3% (w/w) based on the total weight of the pharmaceutical formulation. In some embodiments, the at least one alcohol cosolvent is one alcohol cosolvent selected from the group consisting of ethanol and isopropyl alcohol. In other embodiments, the at least one alcohol cosolvent is a combination or mixture of ethanol and isopropyl alcohol.

In one embodiment, the pharmaceutical formulation includes only one PEGylated lipid selected from the group consisting of DSPE-PEG2000, DSG-PEG2000, and DPG-PEG2000.

In one embodiment, the only one PEGylated lipid is present at a concentration of from about 0.2 to about 3 mg/mL. In another embodiment, the only one PEGylated lipid is present at a concentration of from about 0.2 to about 0.5 mg/mL. In another embodiment, the only one PEGylated lipid is present at a concentration of from about 0.25 to about 0.75 mg/mL. In another embodiment, the only one PEGylated lipid is present at a concentration of about 0.5 mg/mL. In another embodiment, the only one PEGylated lipid is present at a concentration of about 0.25 mg/mL. In one embodiment, the only one PEGylated lipid is DSPE-PEG2000.

In one embodiment, the pharmaceutical formulation includes only one surfactant selected from the group consisting of PEG400, PEG1000, and propylene glycol. In one embodiment, the only one surfactant is present at a concentration of from about 0.75 to about 6 mg/mL. In another embodiment, the only one surfactant is present at a concentration of from about 1.5 to about 3 mg/mL. In another embodiment, the only one surfactant is present at a concentration of about 3 mg/mL. In another embodiment, the only one surfactant is present at a concentration of about 1.5 mg/mL. In one embodiment, the only one surfactant is PEG400.

In one embodiment, the pharmaceutical formulation includes only one hydrofluoroalkane propellant selected from the group consisting of HFA134a, HFA227ea, and HFA152a. In one embodiment, the only one hydrofluoroalkane propellant is HFA134a. In another embodiment, the pharmaceutical formulation includes more than one hydrofluoroalkane propellant and consists of a double or triple combination of HFA134a, HFA227ea, and HFA152a.

In one embodiment, the pharmaceutical formulation includes only one alcohol cosolvent selected from the group consisting of ethanol and isopropyl alcohol. In one embodiment, the only one alcohol cosolvent is present at a concentration of from about 3% to about 10% (w/w) based on the total weight of the pharmaceutical formulation. In another embodiment, the only one alcohol cosolvent is present at a concentration of from about 5% to about 10% (w/w) based on the total weight of the pharmaceutical formulation. In another embodiment, the only one alcohol cosolvent is present at a concentration of from about 3% to about 5% (w/w) based on the total weight of the pharmaceutical formulation. In another embodiment, the only one alcohol cosolvent is present at a concentration of about 10% (w/w) based on the total weight of the pharmaceutical formulation. In another embodiment, the only one alcohol cosolvent is present at a concentration of about 9% (w/w) based on the total weight of the pharmaceutical formulation. In another embodiment, the only one alcohol cosolvent is present at a concentration of about 7% (w/w) based on the total weight of the pharmaceutical formulation. In another embodiment, the only one alcohol cosolvent is present at a concentration of about 5% (w/w) based on the total weight of the pharmaceutical formulation. In another embodiment, the only one alcohol cosolvent is present at a concentration of about 3% (w/w) based on the total weight of the pharmaceutical formulation.

In one embodiment, the only one alcohol cosolvent is isopropyl alcohol.

In one embodiment, the pharmaceutical formulation consists essentially of 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation consists essentially of 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation consists essentially of 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

In another embodiment, the pharmaceutical formulation consists of 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation consists of 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a. In a further embodiment, the pharmaceutical formulation consists of 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

The pharmaceutical formulations of the present disclosure may exist in the form of a solution or a suspension of the compound of Formula (I), (Ia), (Ib), (II), or (III), or an enantiomer, diastereomer, or pharmaceutically acceptable salt thereof, and are suitable for pulmonary administration, i.e., for delivery to the lungs of a subject or a patient via inhalation by using, for example, a metered dose inhaler (MDI). The alcohol cosolvent(s) are miscible with the propellant(s) in the formulations in the amounts disclosed herein. Polyoxyethylene (20) cetylether, PEGylated lipids, and surfactants serve to stabilize the formulations and lubricate the valve components of an MDI. In one embodiment, the pharmaceutical formulations of the present disclosure are prepared by first dissolving or dispersing the powdered compound of Formula (I), (Ia), (Ib), (II), or (III), or an enantiomer, diastereomer, or pharmaceutically acceptable salt thereof, in the liquefied hydrofluoroalkane propellant(s), followed by addition of other ingredients, e.g., polyoxyethylene (20) cetylether or one or more PEGylated lipids, one or more surfactants, and one or more alcohol cosolvents described above. In another embodiment, the pharmaceutical formulations of the present disclosure are prepared by dissolving or dispersing the powdered compound of Formula (I), (Ia), (Ib), (II), or (III), or an enantiomer, diastereomer, or pharmaceutically acceptable salt thereof directly in a mixture of the liquefied hydrofluoroalkane propellant(s) and the other ingredients. The formulations may then be filled into aerosol containers equipped with metering valves and dispensed by MDIs. Canisters generally comprise a container capable of withstanding the vapor pressure of the HFA propellant(s), such as plastic or plastic-coated glass bottle or a metal can, for example, an aluminum can which may optionally be anodized, lacquer-coated and/or plastic-coated. The container is closed with a metering valve. In one embodiment, the canisters are coated with a fluorocarbon polymer, for example, fluorinated ethylene propylene (FEP), polytetrafluoroethylene (PTFE), or a co-polymer of polyethersulphone (PES) and PTFE, as described in WO 96/32151, incorporated herein by reference in its entirety. In one embodiment, the fluorocarbon polymer for coating is FEP. In another embodiment, the fluorocarbon polymer for coating is PTFE. In another embodiment, the canisters are coated with both FEP and PTFE.

The metering valves are designed to deliver a metered amount of the formulation per actuation and include a gasket to prevent leakage of propellant through the valve. The gasket may comprise any suitable elastomeric material, such as low density polyethylene, chlorobutyl, black and white butadiene-acrylonitrile rubbers, butyl rubber, neoprene, EPDM (a polymer of ethylenepropylenediene monomer, as described in WO95/02651, incorporated herein by reference in its entirety), and TPE (thermoplastic elastomer, as described in WO92/11190, incorporated herein by reference in its entirety). Suitable valves are commercially available from manufacturers well known in the aerosol industry, for example, from AptarGroup, U.S., Valois, France (e.g., DF10, DF30, DF31, and DF60), Bespak plc, UK (e.g., BK300, BK356, and BK357) and 3M-Neotechnic Ltd, UK (e.g., Spraymiser™)

The pharmaceutical formulations of the present disclosure in one embodiment, are prepared and filled into canisters in bulk by the following method. First, a metering valve is crimped onto an aluminum can to form an empty canister. Then, the compound of Formula (I), (Ia), (Ib), (II), or (III), or an enantiomer, diastereomer, or pharmaceutically acceptable salt thereof is added to a charge vessel, and a mixture of the alcohol cosolvent(s), surfactant(s), polyoxyethylene (20) cetylether or PEGylated lipid(s), and liquefied propellant(s) is pressure filled through the charge vessel into a manufacturing vessel. An aliquot of the pharmaceutical formulation is then filled through the metering valve into the canister. Typically, in batches prepared for pharmaceutical use, each filled canister is check-weighed, coded with a batch number, and packed into a tray for storage before release testing. In an alternative process, an aliquot of the liquified formulation is added to an open canister under conditions which are sufficiently cold that the formulation does not vaporize, and then a metering valve is crimped onto the canister. In an alternative process, an aliquot of the compound of Formula (I), (Ia), (Ib), (II), or (III), or an enantiomer, diastereomer, or pharmaceutically acceptable salt thereof dissolved in the alcohol cosolvent(s), surfactant(s), and polyoxyethylene (20) cetylether or PEGylated lipid(s) is dispensed into an empty canister, a metering valve is crimped on, and then the HFA propellant(s) are filled into the canister through the valve.

In one embodiment, each filled canister is fitted into a suitable channeling device to form a metered dose inhaler. Suitable channeling devices comprise, for example, a valve actuator and a cylindrical or cone-like passage through which the pharmaceutical formulation may be delivered from the filled canister via the metering valve to the nose or mouth of a patient, e.g., a mouthpiece actuator. The valve stem in one embodiment is situated in a nozzle block which has an orifice leading to an expansion chamber. The expansion chamber has an exit orifice which extends into the mouthpiece. The actuator (exit) orifice diameter in the range of from about 0.1 to about 0.5 mm, from about 0.2 to about 0.4 mm, from about 0.1 to about 0.3 mm, or from about 0.2 to about 0.3 mm is suitable to result in a desirable FPF and low throat deposition of the aerosol of the pharmaceutical formulation.

In one embodiment, the pharmaceutical formulations of the present disclosure are delivered to the lungs of a subject or patient via inhalation by using metered dose inhalers (MDIs).

The MDIs comprise canisters containing the pharmaceutical formulations and utilize the liquefied propellant(s) to expel droplets containing the pharmaceutical formulations to the respiratory tract of the subject or patient as an aerosol.

Exemplary MDIs suitable for delivering the pharmaceutical formulations provided herein include the devices described in the following paragraphs, as well as the MDIs described in U.S. Pat. Nos. 6,170,717, 6,405,727, 3,565,070, 6,328,035, 5,544,647, and 6,155,251, EP Patent No. 0147028B1, CA2298448, and international patent application publications WO1992/009232, WO2003/053501, WO2004/041339, WO2004/041340, WO2001/049350, and WO2004/082633, each of which is herein incorporated by reference in its entirety.

Autohaler® (3M) is an MDI activated by breath and therefore does not require hand-breath coordination to inhale the aerosol of a medication. See U.S. Pat. No. 6,120,752, incorporated herein by reference in its entirety.

Asmair® (Bang and Olufsen Medicom A S) is an MDI that features an integrated dose-counting device and an assisted firing mechanism, making it easier for patients to use.

Easi-Breathe® (Ivax) is a breath-actuated metered dose inhaler. See WO2001/093933, and U.S. Pat. No. 5,447,150, each of which is incorporated herein by reference in its entirety.

Tempo™ (MAP Pharma) is a compact MDI that uses a standard aerosol canister and metering valve. This MDI provides an aerosol flow-control chamber and a synchronized triggering mechanism. See U.S. Pat. Nos. 6,095,141, 6,026,808, and 6,367,471, each of which is incorporated herein by reference in its entirety.

Xcelovent™ (Meridica) is a breath-operated MDI featuring a dose counter. See WO1998/052634, incorporated herein by reference in its entirety.

K-Haler® (Clinical Designs) is a breath-actuated MDI. When the device is in use, the dose is actuated into a kinked tube. The kinked tube is straightened by a breath operated lever, resulting in release of the dose.

MD Turbo™ (Respirics) is a breath-actuated inhaler.

Spacehaler™ (Celltech Mediva) is a compact, low velocity spray pressurized MDI.

eMDI™ developed by H&T Presspart and Cohero Health is the first market-ready, fully embedded and connected metered-dose inhaler. With embedded sensors, mechanical or electronic dose counting and display, the device is capable of tracking and recording data on the use of medications, and sharing it with patients and physicians via a mobile or web app. As a result, the patients receive real-time updates on medication use and alerts, and the physicians can develop treatment plans based on the complete, objective data provided by the device.

The canisters filled with the pharmaceutical formulations and the metered dose inhalers comprising the filled canisters described herein constitute further aspects of the present disclosure. Emitted dose is defined as the amount of an active pharmaceutical ingredient released from an inhaler device. In one embodiment, the MDI is configured to have an emitted dose of from about 30 to about 70 μg of the compound of Formula (I), (Ia), (Ib), (II), or (III), or an enantiomer, diastereomer, or pharmaceutically acceptable salt thereof. In a further embodiment, the MDI is configured to have an emitted dose of from about 30 to about 70 μg of the compound of Formula (I), (Ia), (Ib), (II), or (III), or pharmaceutically acceptable salt thereof. In a further embodiment, the MDI is configured to have an emitted dose of from about 30 to about 70 μg of the compound of Formula (I), (Ia), (Ib), (II), or (III). In another embodiment, the MDI is configured to have an emitted dose of from about 40 to about 60 μg of the compound of Formula (I), (Ia), (Ib), (II), or (III), or an enantiomer, diastereomer, or pharmaceutically acceptable salt thereof. In a further embodiment, the MDI is configured to have an emitted dose of from about 40 to about 60 μg of the compound of Formula (I), (Ia), (Ib), (II), or (III), or pharmaceutically acceptable salt thereof. In a further embodiment, the MDI is configured to have an emitted dose of from about 40 to about 60 μg of the compound of Formula (I), (Ia), (Ib), (II), or (III).

In one embodiment, the MDI is configured to produce an aerosol of the pharmaceutical formulation with a mass median aerodynamic diameter (MMAD) of from about 1 to about 3 μm, as measured by Next Generation Impactor (NGI). In another embodiment, the MDI is configured to produce an aerosol of the pharmaceutical formulation with an MMAD of from about 1 to about 2 μm, as measured by NGI. In another embodiment, the MDI is configured to produce an aerosol of the pharmaceutical formulation with an MMAD of about 1.5 μm, as measured by NGI.

In one embodiment, the MDI is configured to produce an aerosol of the pharmaceutical formulation with a throat deposition of from about 5% to about 40%, as measured by NGI. In another embodiment, the MDI is configured to produce an aerosol of the pharmaceutical formulation with a throat deposition of from about 10% to about 30%, as measured by NGI. In another embodiment, the MDI is configured to produce an aerosol of the pharmaceutical formulation with a throat deposition of from about 10% to about 25%, as measured by NGI. In another embodiment, the MDI is configured to produce an aerosol of the pharmaceutical formulation with a throat deposition of from about 15% to about 25%, as measured by NGI. In another embodiment, the MDI is configured to produce an aerosol of the pharmaceutical formulation with a throat deposition of from about 15% to about 20%, as measured by NGI.

In one embodiment, the MDI is configured to produce an aerosol of the pharmaceutical formulation with a fine particle fraction (FPF) of from about 50% to about 95%, as measured by NGI. In another embodiment, the MDI is configured to produce an aerosol of the pharmaceutical formulation with an FPF of from about 60% to about 85%, as measured by NGI. In another embodiment, the MDI is configured to produce an aerosol of the pharmaceutical formulation with an FPF of from about 70% to about 85%, as measured by NGI. In another embodiment, the MDI is configured to produce an aerosol of the pharmaceutical formulation with an FPF of from about 75% to about 85%, as measured by NGI. In another embodiment, the MDI is configured to produce an aerosol of the pharmaceutical formulation with an FPF of from about 70% to about 80%, as measured by NGI.

“Fine particle dose” or “FPD” refers to the dose, either in total mass or fraction of the nominal dose or metered dose, that is within a respirable range. The dose that is within the respirable range is measured in vitro to be the dose that deposits beyond the throat stage of a cascade impactor, i.e., the sum of dose delivered at stages 3 through filter in a Next Generation Impactor operated at a flow rate of 30 l/min. In one embodiment, the MDI is configured to produce an aerosol of the pharmaceutical formulation with an FPD of from about 10 to about 40 μg, as measured by NGI. In another embodiment, the MDI is configured to produce an aerosol of the pharmaceutical formulation with an FPD of from about 15 to about 40 μg, as measured by NGI.

In another embodiment, the MDI is configured to produce an aerosol of the pharmaceutical formulation with an FPD of from about 20 to about 40 μg, as measured by NGI. In another embodiment, the MDI is configured to produce an aerosol of the pharmaceutical formulation with an FPD of from about 30 to about 40 μg, as measured by NGI. In another embodiment, the MDI is configured to produce an aerosol of the pharmaceutical formulation with an FPD of from about 30 to about 35 μg, as measured by NGI.

In another aspect, the present disclosure provides a method for treating pulmonary hypertension (PH) in a patient in need thereof. The method includes administering an effective amount of the pharmaceutical formulation disclosed herein, i.e., a pharmaceutical formulation comprising a compound of Formula (I), (Ia), (Ib), (II) or (III), or an enantiomer, diastereomer, or pharmaceutically acceptable salt thereof, to the lungs of the patient by inhalation via an MDI. In one embodiment, the pharmaceutical formulation comprises a compound of Formula (I), (Ia), (Ib), (II) or (III), or a pharmaceutically acceptable salt thereof. In another embodiment, the pharmaceutical formulation comprises a compound of Formula (I), (Ia), (Ib), (II) or (III). In one embodiment, the administering includes aerosolizing the pharmaceutical formulation by using an MDI, and administering an aerosolized pharmaceutical formulation to the lungs of the patient via inhalation. In some embodiments, the aerosolized pharmaceutical formulation comprises particles with an MMAD of from about 1 to about 3 μm, from about 1 to about 2 μm, or about 1.5 μm, as measured by NGI. In some embodiments, the aerosolized pharmaceutical formulation has a throat deposition of from about 5% to about 40%, from about 10% to about 30%, from about 10% to about 25%, from about 15% to about 25%, or from about 15% to about 20%, as measured by NGI. In some embodiments, the aerosolized pharmaceutical formulation comprises particles with an FPF of from about 50% to about 95%, from about 60% to about 85%, from about 70% to about 85%, from about 75% to about 85%, or from about 70% to about 80%, as measured by NGI.

The World Health Organization (WHO) has classified PH into five groups. Group 1 PH includes pulmonary arterial hypertension (PAH), idiopathic pulmonary arterial hypertension (IPAH), familial pulmonary arterial hypertension (FPAH), and pulmonary arterial hypertension associated with other diseases (APAH). For example, pulmonary arterial hypertension associated with collagen vascular disease (e.g., scleroderma), congenital shunts between the systemic and pulmonary circulation, portal hypertension and/or HIV infection are included in group 1 PH. Group 2 PH includes pulmonary hypertension associated with left heart disease, e.g., atrial or ventricular disease, or valvular disease (e.g., mitral stenosis). Group 3 pulmonary hypertension is characterized as pulmonary hypertension associated with lung diseases, e.g., chronic obstructive pulmonary disease (COPD), interstitial lung disease (ILD), and/or hypoxemia. Group 4 pulmonary hypertension is pulmonary hypertension due to chronic thrombotic and/or embolic disease. Group 4 PH is also referred to as chronic thromboembolic pulmonary hypertension. Group 4 PH patients experience blocked or narrowed blood vessels due to blood clots. Group 5 PH is the “miscellaneous” category, and includes PH caused by blood disorders (e.g., polycythemia vera, essential thrombocythemia), systemic disorders (e.g., sarcoidosis, vasculitis) and/or metabolic disorders (e.g., thyroid disease, glycogen storage disease).

The methods provided herein can be used to treat group 1 (i.e., pulmonary arterial hypertension or PAH), group 2, group 3, group 4 or group 5 PH patients, as characterized by the WHO. In one embodiment of the methods, the pulmonary hypertension treated is chronic thromboembolic pulmonary hypertension.

In another embodiment of the methods, the pulmonary hypertension treated is pulmonary arterial hypertension (PAH). In some embodiments, the PAH treated is class I PAH, class II PAH, class III PAH, or class IV PAH, as characterized by the New York Heart Association (NYHA).

In one embodiment, the PAH is class I PAH, as characterized by the NYHA.

In another embodiment, the PAH is class II PAH, as characterized by the NYHA.

In yet another embodiment, the PAH is class III PAH, as characterized by the NYHA.

In still another embodiment, the PAH is class IV PAH, as characterized by the NYHA.

In another aspect, the present disclosure provides a method for treating portopulmonary hypertension (PPH) in a patient in need thereof. The method includes administering an effective amount of the pharmaceutical formulation disclosed herein, i.e., a pharmaceutical formulation comprising a compound of Formula (I), (Ia), (Ib), (II) or (III), or an enantiomer, diastereomer, or pharmaceutically acceptable salt thereof, to the lungs of the patient by inhalation via an MDI. In one embodiment, the pharmaceutical formulation comprises a compound of Formula (I), (Ia), (Ib), (II) or (III), or a pharmaceutically acceptable salt thereof. In another embodiment, the pharmaceutical formulation comprises a compound of Formula (I), (Ia), (Ib), (II) or (III). In one embodiment, the administering includes aerosolizing the pharmaceutical formulation by using an MDI, and administering an aerosolized pharmaceutical formulation to the lungs of the patient via inhalation. In some embodiments, the aerosolized pharmaceutical formulation comprises particles with an MMAD of from about 1 to about 3 μm, from about 1 to about 2 μm, or about 1.5 μm, as measured by NGI. In some embodiments, the aerosolized pharmaceutical formulation has a throat deposition of from about 5% to about 40%, from about 10% to about 30%, from about 10% to about 25%, from about 15% to about 25%, or from about 15% to about 20%, as measured by NGI. In some embodiments, the aerosolized pharmaceutical formulation comprises particles with an FPF of from about 50% to about 95%, from about 60% to about 85%, from about 70% to about 85%, from about 75% to about 85%, or from about 70% to about 80%, as measured by NGI.

In some embodiments, the PH, PAH, or PPH patient treated by the disclosed methods manifests one or more of the following therapeutic responses: (1) a reduction in the pulmonary vascular resistance index (PVRI) from pretreatment value, (2) a reduction in mean pulmonary artery pressure from pretreatment value, (3) an increase in the hypoxemia score from pretreatment value, (4) a decrease in the oxygenation index from pretreatment values, (5) improved right heart function, as compared to pretreatment, and (6) improved exercise capacity (e.g., as measured by the six-minute walk test) compared to pretreatment.

In one embodiment of the disclosed methods, the PH, PAH, or PPH patient is administered the pharmaceutical formulation once daily. In another embodiment of the disclosed methods, the PH, PAH, or PPH patient is administered the pharmaceutical formulation twice daily. In another embodiment of the disclosed methods, the PH, PAH, or PPH patient is administered the pharmaceutical formulation three times daily. In still another embodiment of the disclosed methods, the PH, PAH, or PPH patient is administered the pharmaceutical formulation four or more times daily. In one embodiment, the administration is with food. In one embodiment, each administration comprises 1 to 5 doses (puffs) from an MDI, for example, 1 dose (1 puff), 2 doses (2 puffs), 3 doses (3 puffs), 4 doses (4 puffs) or 5 doses (5 puffs). The MDI may be one of those described above.

In still another aspect, the present disclosure provides a method for treating pulmonary fibrosis in a patient in need thereof. The method includes administering an effective amount of the pharmaceutical formulation disclosed herein, i.e., a pharmaceutical formulation comprising a compound of Formula (I), (Ia), (Ib), (II) or (III), or an enantiomer, diastereomer, or pharmaceutically acceptable salt thereof, to the lungs of the patient by inhalation via a metered dose inhaler. In one embodiment, the pharmaceutical formulation comprises a compound of Formula (I), (Ia), (Ib), (II) or (III), or a pharmaceutically acceptable salt thereof. In another embodiment, the pharmaceutical formulation comprises a compound of Formula (I), (Ia), (Ib), (II) or (III). In one embodiment, the administering includes aerosolizing the pharmaceutical formulation with an MDI, and administering an aerosolized pharmaceutical formulation to the lungs of the patient via inhalation. In some embodiments, the aerosolized pharmaceutical formulation comprises particles with an MMAD of from about 1 to about 3 μm, from about 1 to about 2 μm, or about 1.5 μm, as measured by NGI. In some embodiments, the aerosolized pharmaceutical formulation has a throat deposition of from about 5% to about 40%, from about 10% to about 30%, from about 10% to about 25%, from about 15% to about 25%, or from about 15% to about 20%, as measured by NGI. In some embodiments, the aerosolized pharmaceutical formulation comprises particles with an FPF of from about 50% to about 95%, from about 60% to about 85%, from about 70% to about 85%, from about 75% to about 85%, or from about 70% to about 80%, as measured by NGI. The patient, in one embodiment, is administered the pharmaceutical formulation once daily, twice daily, three times daily, or four or more times daily.

In one embodiment, the administration is with food. In one embodiment, each administration comprises 1 to 5 doses (puffs) from an MDI, for example, 1 dose (1 puff), 2 doses (2 puffs), 3 doses (3 puffs), 4 doses (4 puffs) or 5 doses (5 puffs). The MDI may be one of those described above.

In still another aspect, the present disclosure provides a system for treating pulmonary hypertension, portopulmonary hypertension, or pulmonary fibrosis. The system includes the pharmaceutical formulation disclosed herein, i.e., a pharmaceutical formulation comprising a compound of Formula (I), (Ia), (Ib), (II) or (III), or an enantiomer, diastereomer, or pharmaceutically acceptable salt thereof, and an MDI. In one embodiment, the pharmaceutical formulation comprises a compound of Formula (I), (Ia), (Ib), (II) or (III), or a pharmaceutically acceptable salt thereof. In another embodiment, the pharmaceutical formulation comprises a compound of Formula (I), (Ia), (Ib), (II) or (III). The MDI may be one of those described previously.

### Examples

The present invention is further illustrated by reference to the following Examples. However, it should be noted that these Examples, like the embodiments described above, are illustrative and are not to be construed as restricting the scope of the invention in any way.

### Materials

Metering valves: Aptar DF 316/50 RCU CS20 ARGENT

### MDI Cannisters

a. Presspart plain (un-coated) aluminum canisters (C0128-000, 19 mL Plain)
b. Kimble chase PVC coated serum bottles, 20 mL, 20 mm; Part No. 9610000023)

### Actuators: Presspart Actuators

a. OD/JL=0.30/0.7, Part No. NM200DIS ØT3, 16A ØS0,3 JL0,7 BL/AZ AN(S)
b. OD/JL=0.27/0.5, Part No. NM120DC ØT3, 16A(N3) ØS0,27 JL0,5 BL/296C
c. OD/JL=0.25/0.5, Part No. NM120DC ØT3, 16A(N3) ØS0,25 JL0,5 BL/296C
d. OD/JL=0.20/0.5, Part No. NM120DC ØT3, 16A(N3) ØS0,2 JL0,5 BL/296C

### Propellants:

a. HFA134a, CAS No. 811-97-2
   Mexichem Fluor—Zephex 134a
   Daikin—Solkane 134a
b. HFA227ea, CAS No. 431-89-0
   Mexichem Fluor—Zephex 227ea
   Daikin Solkane 227ea, CAS NO. 431-89-0
c. HFA152a, CAS No. 75-37-6
   Chemours HP-152

### Active Pharmaceutical Ingredients (APIs)

Treprostinil palmitil (C16TR)

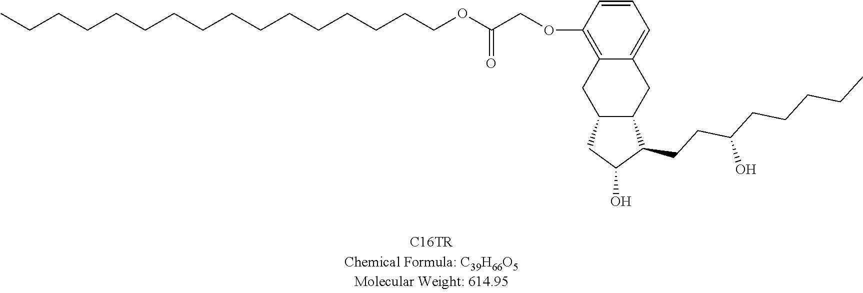 [SMILES: CCCCCCCCCCCCCCCCOC(=O)COc1cccc2c1C[C@H]1C[C@@H](O)[C@H](CC[C@@H](O)CCCCC)[C@H]1C2]

Dodecyl treprostinil (C12TR); Manufacturer: Insmed

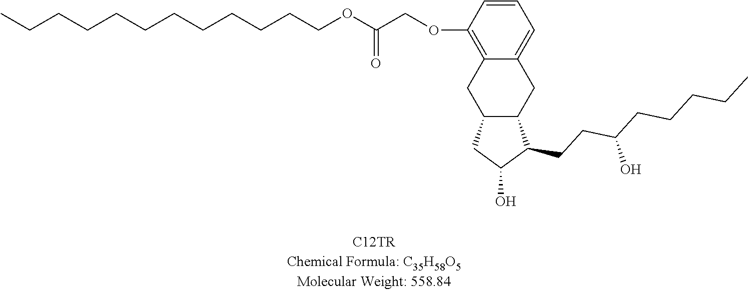 [SMILES: CCCCCCCCCCCCOC(=O)COc1cccc2c1C[C@H]1C[C@@H](O)[C@H](CC[C@@H](O)CCCCC)[C@H]1C2]

### Excipients

Brij® 58; Manufacturer: Sigma Aldrich; Product Number: P-5884

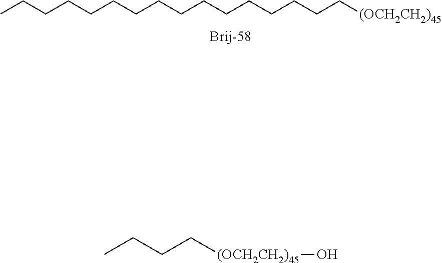

DSPE-PEG2000 (alternative name: DSPE-P2K); Manufacturer: NOF America; Product Number: DSPE-020CN

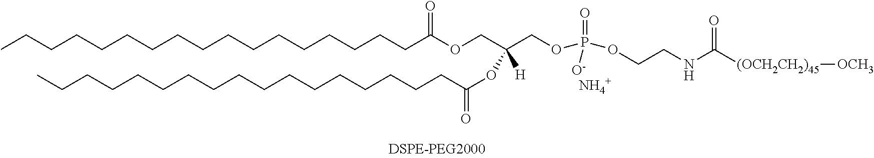

MSPE-PEG2000 (alternative name: MSPE-P2K); Manufacturer: NOF America; Product Number: MSPE-020CN

### Co-solvents

Isopropanol; Manufacturer: Fisher; Product Number: A464-4; Assay=>99.9%; H₂O=<0.2%; CAS: 67-63-0
Ethanol, absolute; Manufacturer: Fisher; Product Number: BP2818-4; Assay=>99.5%; H₂O=<0.2%; CAS: 64-17-5

### Example 1—Pharmacokinetic Evaluations on Inhaled C16TR-MDI Formulations in Rats

The treprostinil prodrug hexadecyl treprostinil (also referred to as treprostinil palmitil or C16TR) is a long-acting pulmonary vasodilator. C16TR has previously been formulated in a lipid nanoparticle for inhaled delivery by nebulization. The nebulized C16TR formulation (termed INS1009) contains C16TR and the excipients squalane and DSPE-PEG2000 at a molar ratio of 45:45:10, suspended in PBS (see Corboz et al., J Pharmacol Exp Ther. 363:348-357 (2017), incorporated herein by reference in its entirety). Pulmonary vasodilation is associated more with local activity of TRE within the lungs and less with the level of TRE in the plasma (see Chapman R W et al., Pulm. Pharmacol. Ther. 49:104-111 (2018), incorporated herein by reference in its entirety). Low concentration of TRE in the plasma would minimize potential systemic adverse events such as reductions in systemic blood pressure. This example describes development of metered dose inhaler (MDI) formulations of C16TR (C16TR-MDI) and evaluation of eighteen of those formulations for their PK profile when delivered by nose-only inhalation to rats, in parallel with nebulized INS1009 and a C16TR dry powder formulation. The C16TR dry powder formulation used in this example includes C16TR, DSPE-PEG2000, mannitol, and leucine in a weight ratio of 1.5:0.75:70:30.

### Methods and Materials

### 1. Preparation of C16TR-MDI Formulations

A single feedstock containing each excipient at the appropriate concentrations was prepared by combining the solid ingredients (C16TR and PEGylated lipid, e.g., DSPE-PEG2000) in the appropriately sized glassware, adding the appropriate amount of a surfactant, e.g., PEG400, and then diluting to volume with a desired alcohol cosolvent (e.g., EtOH or isopropyl alcohol (IPA)). The solution was then placed on an incubated shaker set to 40° C. and −125 rpm until the solution became homogeneous, typically about 10 minutes.

MDI canisters were then filled by adding the appropriate amount of feedstock, crimping a valve in place, and adding hydrofluoroalkane (HFA) propellant using a Pamasol Lab-2016 manual filling station. Crimp height was set based on valve manufacturer specs (5.71 mm for the Aptar DF 316/50 RCU CS20 ARGENT valve). Crimp heights were measured using a Socoge International Crimper-Control (model 020:743-03-143). Mass measurements were made throughout the manufacturing process to calculate can compositions. All concentration calculations were made based on mass measurements. The feedstock composition was reported in mg/g for each component. By weighing the canisters before and after addition of any components, after crimping, and after addition of propellant, the exact can composition was calculated. The density of individual components was used to do a unit conversion, and concentration in mg/mL rather than mg/g was reported herein. Table 1 summarizes the targeted compositions of the C16TR-MDI formulations prepared.

TABLE 1
Targeted compositions of different C16TR-MDI formulations

| | | [DSPE- | [DSG- | [DPG- | | | [Propylene | | | | | |
|---|---|---|---|---|---|---|---|---|---|---|---|---|
| | [C16TR] | P2K] | P2K] | P2K] | [Brij-58] | [PEG400] | Glycol] | [PEG1000] | | | | |
| Formulation | mg/mL | mg/mL | mg/mL | mg/mL | mg/mL | mg/mL | mg/mL | mg/mL | % R—OH | | HFA | Slope |
| MDI-A | 1 | 0.5 | — | — | — | — | — | — | 9 | EtOH | 134 | −0.0583 |
| MDI-B | 0.5 | 0.25 | — | — | — | 0.75 | — | — | 3 | EtOH | 227 | −0.1191 |
| MDI-C | 0.5 | 0.25 | | | | 0.75 | | | 5 | EtOH | 227 | NA |
| MDI-D | 1* | 0.5 | — | — | — | — | — | — | 10 | EtOH | 227 | NA |
| MDI-E | 3 | 3 | — | — | — | — | — | — | 10 | EtOH | 227 | −0.1127 |
| MDI-F | 1 | 0.5 | — | — | — | 3 | — | — | 5 | EtOH | 227 | −0.1197 |
| MDI-G | 0.5 | 0.25 | — | — | — | 1.5 | — | — | 3 | EtOH | 227 | −0.1151 |
| MDI-H | 1 | 0.5 | — | — | — | — | 1.5 | — | 3 | EtOH | 227 | −0.0815 |
| MDI-I | 1 | 0.5 | — | — | — | — | — | 3 | 3 | EtOH | 227 | −0.0707 |
| MDI-J | 0.5 | — | 0.25 | — | — | 3 | — | — | 5 | EtOH | 227 | −0.159 |
| MDI-K | 0.5 | — | — | 0.25 | — | 3 | — | — | 5 | EtOH | 227 | −0.1148 |
| MDI-L | 0.5 | 0.25 | — | — | — | 3 | — | — | 5 | EtOH | 227 | −0.119 |
| MDI-M | 1 | 0.5 | — | — | — | 6 | — | — | 5 | EtOH | 227 | −0.1044 |
| M-Repeat | 1 | 0.5 | — | — | — | 6 | — | — | 5 | EtOH | 227 | −0.1826 |
| MDI-N | 0.5 | 0.25 | — | — | — | 6 | — | — | 5 | EtOH | 227 | −0.112 |
| MDI-R | 1 | — | — | — | — | 6 | — | — | 5 | EtOH | 227 | −0.1207 |
| MDI-S | 1 | — | — | — | 0.5 | 6 | — | — | 5 | EtOH | 227 | −0.1214 |
| MDI-T | 1 | — | — | — | 0.5 | 3 | — | — | 6.8 | IPA | 134 | −0.1379 |
| MDI-W high | 1 | 0.5 | — | — | — | 3 | — | — | 10 | IPA | 134 | −0.1156 |
| MDI-W low | 1 | 0.5 | — | — | — | 3 | — | — | 10 | IPA | 134 | −0.151 |
| MDI-X | 1 | 0.5** | — | — | — | 3 | — | — | 10 | IPA | 134 | −0.1074 |

*For MDI-D, C12TR was used instead of C16TR.
**DSPE-P2K containing 10 mol % MSPE-PEG2K and 10 mol % stearic acid.
MIDI-W high denotes the MDI-W formulation administered to rats by inhalation at a high dose of 115 μg/kg body weight.
MIDI-W low denotes the MDI-W formulation administered to rats by inhalation at a low dose of 62.2 μg/kg body weight.

### 2. Nose Only Inhalation, Blood and Lung Sampling

Nose-only inhalation studies in rats were performed using an inhalation tower modified for MDI delivery. For each study, the weight of the canisters was measured before and after study and the duration of actuation was recorded.

Male Sprague-Dawley rats from Charles River Laboratories (St Constant, Quebec, Canada) weighing between 300 g and 350 g at the start of dosing were used in the study. On the day of study, the rats were placed in restraining tubes which were connected to outlet ports in a 12-animal nose-only inhalation chamber. Cohorts of 11 rats were used with a filter connected to the one remaining outlet port from which the aerosol concentration in the nose-only chamber was measured. Air was circulated through the chamber at a flow rate of 20 L/min. A vacuum pump was connected to the filter and set at a vacuum flow of either 2.0 or 3.0 L/min which began at 5 min after the start of the aerosolization of the formulation and ended 1, 2, or 3 min later, i.e. filter sampling time of 1, 2, or 3 min depending on the formulation concentration and the number of canisters used. The filter samples were analyzed for C16TR measured by IPLC with a charged aerosol detector (CAD).

Following exposure to the formulations, blood and lung tissue samples were obtained from each rat at times of 0.5, 2, 4, 6, 12 and 24 h for blood and 0.5, 6, 12 and 24 h for lungs. The 0.5 h time point was defined as the immediate post dose (IPD) sampling time. The plasma was separated from the blood samples and the lung tissues homogenized to measure the concentrations of TRE and C16TR by IPLC MS/MS. The concentrations of C16TR and TRE in the lungs were expressed as either their absolute values or combined into a single value and expressed as the C16TR equivalent value (C16TReq). The conversion of TRE to molar equivalents of C16TR involved multiplication of the TRE concentration by a factor of 1.575 and is based upon the molecular weights of C16TR and TRE which are 614.9 and 390.5 g/mol, respectively.

### 3. Pharmacokinetic Analysis

Analysis of the plasma and lung pharmacokinetics was performed with PKSolver program which is an add-in program for Microsoft Excel. Using the module of “Non-Compartmental Analysis after Extravascular Input”, several PK parameters shown in Table 2 were calculated.

TABLE 2
Pharmacokinetic parameters derived from the lung and plasma
concentrations of C16TR and TRE in the lungs and plasma

| | Units | | |
|---|---|---|---|
| Parameter | Plasma | Lung | Definition |
| AUC_{0-inf} | ng*h/mL | ng*h/g | Area under the concentration-time curve from zero up |
| | | | to infinity with extrapolation of the terminal phase. |
| AUC₀₋ₜ_{_{—}}_{obs} | ng*h/mL | ng*h/g | Area under the concentration-time curve from IPD to |
| | | | the final timepoint; the data has not been extrapolated |
| | | | to infinity. |
| Cₘₐₓ | ng/mL | ng/g | Maximum concentration after administration. |
| CL/F | L/h | L/h | Apparent clearance of drug after drug administration |
| | | | (CL/F = Dose/AUC_{0-inf}) |
| Lambda (λ) | h⁻¹ | h⁻¹ | Terminal rate constant (slowest rate constant of the disposition). |
| Z | | | |
| t½ | h | h | Terminal half-life (In2/ λz = 0.693/λz) |
| Tₘₐₓ | h | h | Time to reach Cₘₐₓ |
| Vz/F | L | g | Apparent volume of distribution during the terminal |
| | | | phase after drug administration (Vz/F = Dose/AUC_{0-inf}_{.} λz) |

### 4. Delivered Dose Calculations

The delivered inhaled dose was calculated using the relationships of aerosol drug concentration, duration of exposure, respiratory minute volume, deposition fraction and body weight as previously described by Alexander D J et al., Inhal. Tox. 20:1179-1189 (2008), incorporated herein by reference in its entirety.

### Results

### 1. Delivered Dose

The lowest delivered dose was observed for MDI-N(2.86 μg/kg) while the highest delivered dose was observed for MDI-T (501.2 μg/kg). The disparity in delivered dose may be in part due to differences in chamber efficiency and aerosol performance between the different MDI formulations.

### 2. Lung Pharmacokinetics

Each of the eighteen inhaled C16TR-MDI formulations demonstrated a first order exponential decay in lung C16TR and TRE over 24 h with elimination kinetics comparable to that seen with nebulized INS1009. The lung C16TReq Cₘₐₓ and AUC_{0-inf} values were highest for MDI-T and lowest for MDI-S, however, the delivered dose quantified from the filter data varied greatly between the different MDI formulations. The Tₘₐₓ was identical for each of the C16TR-MDI formulations tested. For the formulation MDI-W, Lung C16TReq has a calculated half-life of 8.01 hours when dosed at 62.2 μg/kg and 11.29 hours when dosed at 115 μg/kg.

### 3. Plasma Pharmacokinetics

Each of the eighteen inhaled C16TR-MDI formulations demonstrated a first order exponential decay in plasma TRE over 24 h with elimination kinetics comparable to that seen with nebulized INS1009. Interestingly, the calculated plasma T ½ for these formulations varies from a minimum of 1.76 hours for MDI-M-Repeat to a maximum of 12.51 hours for MDI-S. For the formulation MDI-W, plasma TRE has a calculated half-life of 8.47 hours when dosed at 62.2 μg/kg and 7.13 hours when dosed at 115 μg/kg. The plasma Tₘₐₓ was very consistent at 0.5 hours for all but three formulations (MDI-E, MDI-G, and MDI-J). MDI-E, MDI-G, and MDI-J had Tₘₐₓ values of 2.0 hours. The results of this example show that each of the C16TR-MDI formulations exhibited a similar PK profile to nebulized INS1009 with the highest levels of C16TR and TRE found in the lungs immediately after dosing (0.5 h), with an exponential decay over 24 h, and a half-life of approximately 8 h. C16TR-MDI-T, C16TR-MDI-H, C16TR-MDI-W (high dose) had relatively high levels of C16TR and TRE in the lungs immediately after dosing, although a much higher inhaled dose of C16TR-MDI-T was given (501.2 μg/kg) compared to C16TR-MDI-H (57.3 μg/kg) and C16TR-MDI-W high dose (115.0 μg/kg). These compounds maintained high levels of both C16TR and TRE in the lungs at 24 h after dosing. All these compounds had relatively low level of TRE in the plasma both immediately after dosing and over the 24 h collection period. Of the eighteen C16TR-MDI formulations, C16TR-MDI-T had one of the highest levels of C16TR in the lungs immediately after dosing with low TRE concentration in the plasma. This may be important to limit adverse events such as falls in systemic blood pressure that would occur with high TRE levels in the plasma. The pulmonary dose varied significantly between formulations due to variations in MDI canister composition and delivery efficiency of the inhalation tower.

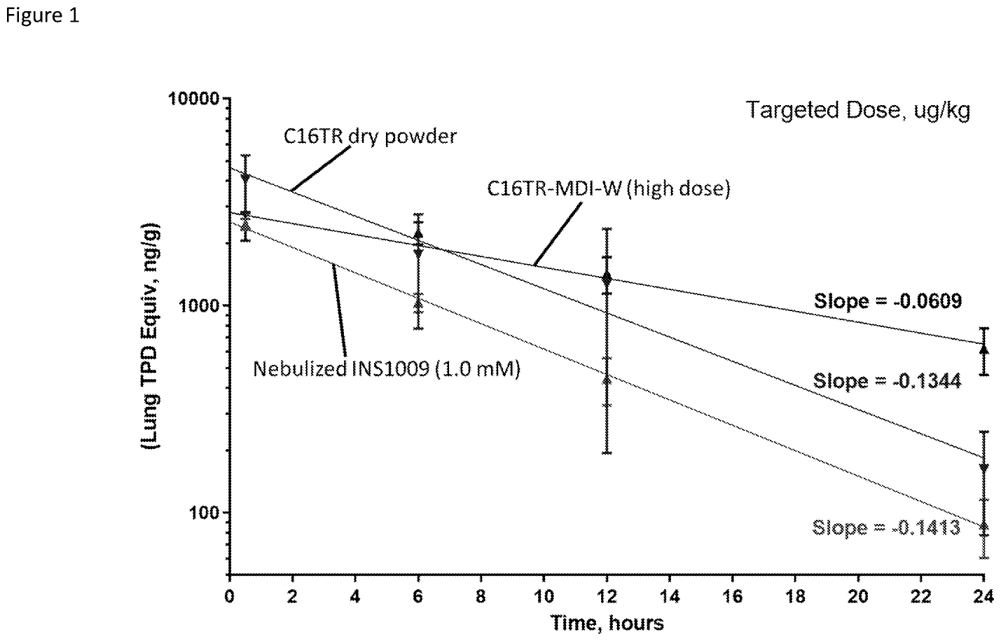

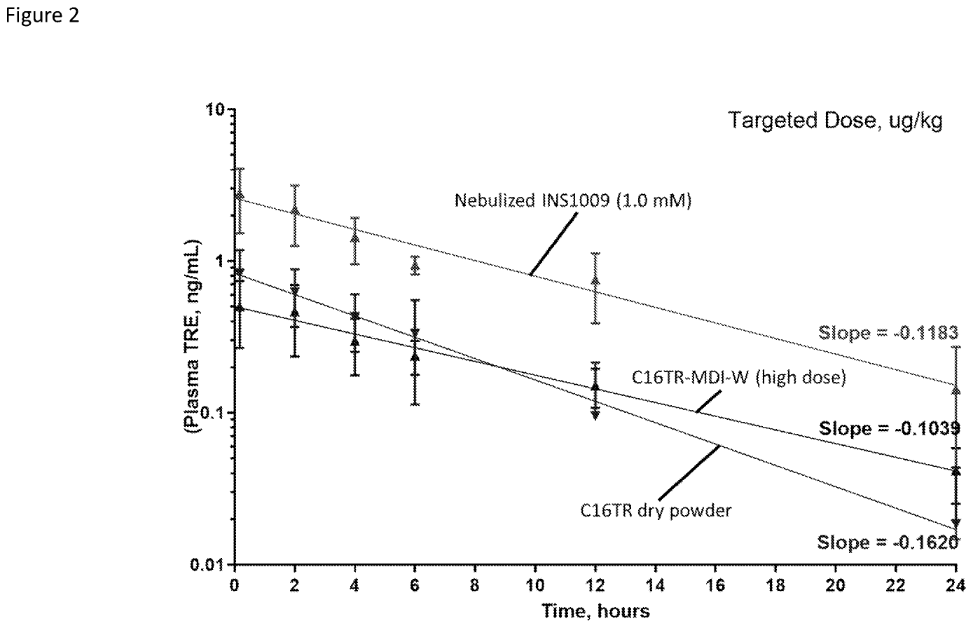

When compared to that of nebulized INS1009 and C16TR dry powder formulations, the PK profile of C16TR-MDI formulations appears comparable for lung and plasma measurements, as shown in FIG. 1 and FIG. 2. Additionally, the PK of MI-K and MI-L are very similar to that of nebulized INS1009 (slope=−0.115 for MDI-K, −0.119 for MDI-L, and −0.1413 for Nebulized-INS1009). Compared to MDI-W, MI-K and MDI-L are formulated at low concentration (0.5 mg/mL C16TR) and use ethanol as the alcohol co-solvent. Thus, MI-W appears to have balanced characteristics with respect to chemical stability (with the use of IPA as co-solvent), physical stability (10% w/w alcohol co-solvent), in vivo performance (PK as well as efficacy and cough data discussed in the following examples). Interestingly, there appears to be a significant difference in the lung C16TR_{eq}: plasma TRE Cₘₐₓ ratio for the different formulations. Typical lung/plasma Cₘₐₓ ratio for nebulized INS1009 is ˜800 while the ratio for C16TR dry powder formulations ranges from ˜1,600-13,000. For C16TR-MDI-W, we observed an intermediate lung/plasma Cₘₐₓ ratio of ˜4,100 when delivered at a low dose (62.2 μg/kg) and ˜4,800 when delivered at a high dose (115 μg/kg). This indicates progressively lower systemic exposure as we move from nebulized INS1009 to C16TR dry powder formulations to C16TR MDI formulations.

### Example 2—Evaluation of Physical Stability of the C16TR-MDI-W Formulation

C16TR is insoluble in all commercially available propellants (HFA134a, HFA227ea, and HFA152a) and requires the use of alcohol co-solvent. When formulated with DSPE-PEG2000 and PEG400 for use with an MDI as well as desirable in vivo performance, the formulation required approximately 10% of alcohol co-solvent. Such a high level of alcohol co-solvent may lead to reduced chemical stability and aerosol performance. Unexpected improvement in chemical stability was observed when a switch from ethanol (industry standard cosolvent) to isopropyl alcohol (novel excipient) was made, as in the C16TR-MDI-W formulation. Additionally, no signs of long-term physical instability of C16TR-MDI-W formulation were observed. The formulation appeared as a clear solution after 24 h at 15° C. The formulation appeared as a cloudy solution after 4 h at 5° C., but became clear within 30 minutes at room temperature. The formulation appeared as a cloudy solution at −20° C., but was cleared within 30 minutes at room temperature. The formulation became a cloudy solution when stored at −58° C., but turned clear after ˜24 h at room temperature.

### Example 3—Three-Month Accelerated Stability Study of the C16TR-MDI-U and C16TR-MDI-W Formulations

The C16TR-MDI-U and C16TR-MI-W formulations and their respective control formulations without C16TR were subject to a three-month accelerated stability study at 40° C. under ambient humidity conditions. The study included visual observation of glass cans to assess solubility and a chemical stability assay. For chemical stability studies HPLC method was used. The detailed compositions of the sample and control formulations are shown in Table 3 below.

TABLE 3
Compositions of formulations used in three-month accelerated stability study

| Formulation | Composition |
|---|---|
| MDI-W | 1.1 mg/mL C16TR, 0.5 mg/mL DSPE-PEG2000, 3.3 mg/mL |
| | PEG400, 10.7% IPA, HFA134a |
| MDI-W Control | 0.6 mg/mL DSPE-PEG2000, 10.9% IPA, HFA134a |
| MDI-U | 1.0 mg/mL C16TR, 0.5 mg/mL Brij-58, 3.1 mg/mL PEG400, 10.3% |
| | IPA, HFA134a |
| MDI-U Control | 0.5 mg/mL Brij-58, 10.5% IPA, HFA134a |

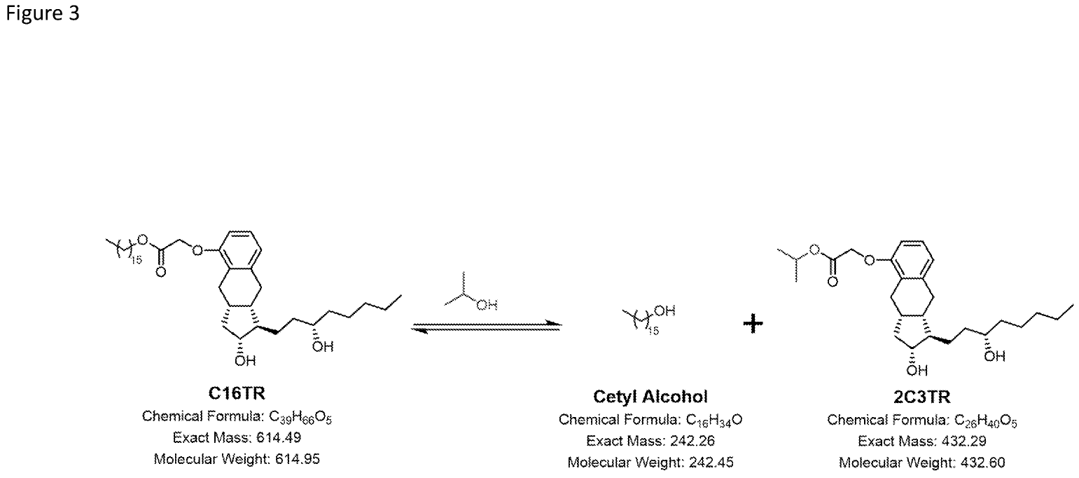

The main degradant of DSPE-PEG2000 was MSPE-PEG2000 which forms when one of the stearoyl chains is cleaved. Transesterification of C16TR with IPA, as shown in FIG. 3, was observed.

No signs of precipitation were observed during the course of this accelerated stability study. Each glass canister presented as a clear, colorless, homogeneous solution.

The chemical stability study results of MI-U at 40° C. over a 3-month period are shown in Table 4. No TRE (treprostinil acid) was detected. However, we observed an increase in 2C3TR, the byproduct of transesterification between C16TR and IPA. In terms of total peak area, the amount of C16TR detected in the samples decreased while the amount of Brij® 58 remained constant. For total degradation there were very low levels present at TO, the highest levels were observed at the 1 M time point, with the 2 M and 3 M samples being intermediate.

TABLE 4
Accelerated stability of the C16TR-MDI-U formulation (40° C.)
Accelerated Stability of MDI-U (40° C.)

| | Condition | | | | | | | |
|---|---|---|---|---|---|---|---|---|
| | T = 0 M | | T = 1 M | | T = 2 M | | T = 3 M | |
| | % Total | | % Total | | % Total | | % Total | |
| Peak | Ave | StDev | Ave | StDev | Ave | StDev | Ave | StDev |
| C16TR | 65.9 | 0.5 | 61.75 | 0.38 | 63.96 | 0.53 | 62.92 | 1.88 |
| 2C3TR | 0.17 | 0.07 | 1.32 | 0.04 | 1.01 | 0.07 | 0.76 | 0.11 |
| Brij | 34.1 | 0.5 | 34.99 | 0.18 | 34.26 | 0.94 | 35.63 | 1.87 |
| RRT~0.812 | 1.16 | 0.02 | 2.47 | 0.19 | 0.63 | 0.02 | 0 | 0 |
| Other | 0 | 0 | 1.74 | 0.08 | 1.93 | 0.21 | 0.95 | 0.2 |
| Total Degradation | 0.06 | 0.1 | 5.53 | 0.21 | 3.57 | 0.22 | 1.71 | 0.23 |

We observed a similar stability pattern for the MDI-U control samples at 40° C. over a 3-month period (Table 5). We observed a decrease in Brij® 58 that was matched by an increase in unknown peaks; many of which can be attributed to Brij® 58 degradation. Interestingly, the highest levels of degradation were observed for the 1 M time point. For the 2 M and 3 M time points degradation appeared to stabilize at approximately 5% (based on CAD peak area).

TABLE 5
Accelerated stability of the C16TR-MDI-U control formulation (40° C.)
Accelerated Stability of MDI-U Control (40° C.)

| | Condition | | | | | | | |
|---|---|---|---|---|---|---|---|---|
| | T = 0 M | | T = 1 M | | T = 2 M | | T = 3 M | |
| | % Total | | % Total | | % Total | | % Total | |
| Peak | Ave | StDev | Ave | StDev | Ave | StDev | Ave | StDev |
| C16TR | 0 | 0 | 0 | 0 | 0 | 0 | 0 | 0 |
| 2C3TR | 0 | 0 | 0 | 0 | 0 | 0 | 0 | 0 |
| Brij | 98.76 | 1.1 | 92.04 | 0.23 | 95.13 | 0.41 | 95.17 | 0.49 |
| RRT~0.812 | 0.83 | 0.73 | 1.08 | 0.02 | 0.63 | 0.02 | 0 | 0 |
| Other | 0.12 | 0.12 | 8.07 | 0.27 | 4.9 | 0.41 | 4.83 | 0.49 |
| Total Degradation | 0.95 | 0.74 | 9.15 | 0.27 | 5.53 | 0.41 | 4.83 | 0.49 |

The chemical stability of MDI-W at 40° C. over a 3-month period is comparable to that of MDI-U (Table 6). No TRE (treprostinil acid) was detected. We also observed an increase in 2C3TR, the byproduct of transesterification between C16TR and IPA. In terms of total peak area, the amount of C16TR detected in the samples fluctuated with both positive and negative changes being observed at different time points in terms of total peak area. However, the amount of C16TR must be decreasing because the amount of 2C3TR was increasing. DSPE-PEG2000 was the least stable component in the formulation with nearly 10% of the initial DSPE-PEG2000 degraded to the MSPE-PEG2000 hydrolysis product. We also observed a steady increase in other unidentified degradations products.

TABLE 6
Accelerated stability of the C16TR-MIDI-W formulation (40° C.)
Accelerated Stability of MDI-W (40° C.)

| | Condition | | | | | | | |
|---|---|---|---|---|---|---|---|---|
| | T = 0 M | | T = 1 M | | T = 2 M | | T = 3 M | |
| | % Total | | % Total | | % Total | | % Total | |
| Peak | Ave | StDev | Ave | StDev | Ave | StDev | Ave | StDev |
| C16TR | 46.47 | 0.17 | 46.54 | 0.2 | 41.92 | 0.22 | 51.8 | 0.2 |
| 2C3TR | 0 | 0 | 0.11 | 0.002 | 0.73 | 0.12 | 1 | 0.05 |
| DSPE-PEG2000 | 53.41 | 0.13 | 52.8 | 0.24 | 54.67 | 0.35 | 43.77 | 0.1 |
| MSPE-PEG2000 | 0.25 | 0.003 | 0.53 | 0.08 | 4.27 | 0.12 | 4.28 | 0.12 |
| Other | 0 | 0 | 0.27 | 0.03 | 1.79 | 0.59 | 1.95 | 0.31 |
| Total Degradation | 0.25 | 0.00 | 0.91 | 0.09 | 6.79 | 0.61 | 7.23 | 0.34 |

We observed a similar stability pattern for the MDI-W control samples at 40° C. over a 3-month period (Table 7). We observed a decrease in DSPE-PEG2000 that was matched partially by an increase in MSPE-PEG2000 and partially by an increase in unknown peaks. It is not clear if DSPE-PEG2000 degrades to MSPE-PEG2000 and other components, or if DSPE-PEG2000 degrades to MSPE-PEG2000 first and then MSPE-PEG2000 continues to degrade into other compounds.

TABLE 7
Accelerated stability of the C16TR-MDI-W control formulation (40° C.)
Accelerated Stability of MDI-W Control (40° C.)

| | Condition | | | | | | | |
|---|---|---|---|---|---|---|---|---|
| | T = 0 M | | T = 1 M | | T = 2 M | | T = 3 M | |
| | % Total | | % Total | | % Total | | % Total | |
| Peak | Ave | StDev | Ave | StDev | Ave | StDev | Ave | StDev |
| C16TR | 0 | 0 | 0 | 0 | 0 | 0 | 0 | 0 |
| 2C3TR | 0 | 0 | 0 | 0 | 0 | 0 | 0 | 0 |
| DSPE-PEG2000 | 99.5 | 0.23 | 98.9 | 0.04 | 96.25 | 0.48 | 94.86 | 0.42 |
| MSPE-PEG2000 | 0.53 | 0.48 | 0.56 | 0.08 | 1.99 | 0.14 | 2.19 | 0.18 |
| Other | 0 | 0 | 0.58 | 0.04 | 1.79 | 0.1 | 5.14 | 0.42 |
| Total Degradation | 0.53 | 0.48 | 1.14 | 0.09 | 3.78 | 0.17 | 4.65 | 0.66 |

### Example 4—Effect of Hardware Configuration on APSD of the C16TR-MDI-W Formulation

Four different MDI actuators of varying orifice diameters and jet lengths were tested with the NGI on MDI-W, in order to observe how each actuator affected aerosol performance. Three MDI canisters were created using a 50 μl metering valve and a plain 19 mL canister to be used for the duration of this study. Only one actuator was used for each size in order to avoid variances in manufacturing. The size of the Presspart actuators were: 0.3/0.7, 0.27/0.5, 0.25/0.5 and 0.2/0.5 millimeter orifice diameter and millimeter jet length respectively.

The actuator orifice diameters were chosen to vary lower than the original 0.3/0.7 actuator size. The effects of different actuator sizes on MDI-W aerodynamic particle size distribution (APSD) characterization is shown in Table 8.

TABLE 8
The effects of different actuator sizes
on MDI-W APSD characterization

| Actuator | MMAD | MMAD | % FPF (< 5 | FPF | FPD (<5 | FPD |
|---|---|---|---|---|---|---|
| Size (mm) | (μm) | SD | μm) | SD | μm) (μm) | SD |
| 0.3/0.7 | 1.69 | 0.04 | 33.5 | 2.3 | 14.71 | 0.69 |
| 0.27/0.5 | 1.72 | 0.04 | 56.1 | 0.9 | 25.7 | 1.48 |
| 0.25/0.5 | 1.61 | 0.1 | 61.8 | 3.6 | 27.49 | 0.41 |
| 0.2/0.5 | 1.54 | 0.03 | 74.2 | 0.9 | 34.93 | 1.28 |

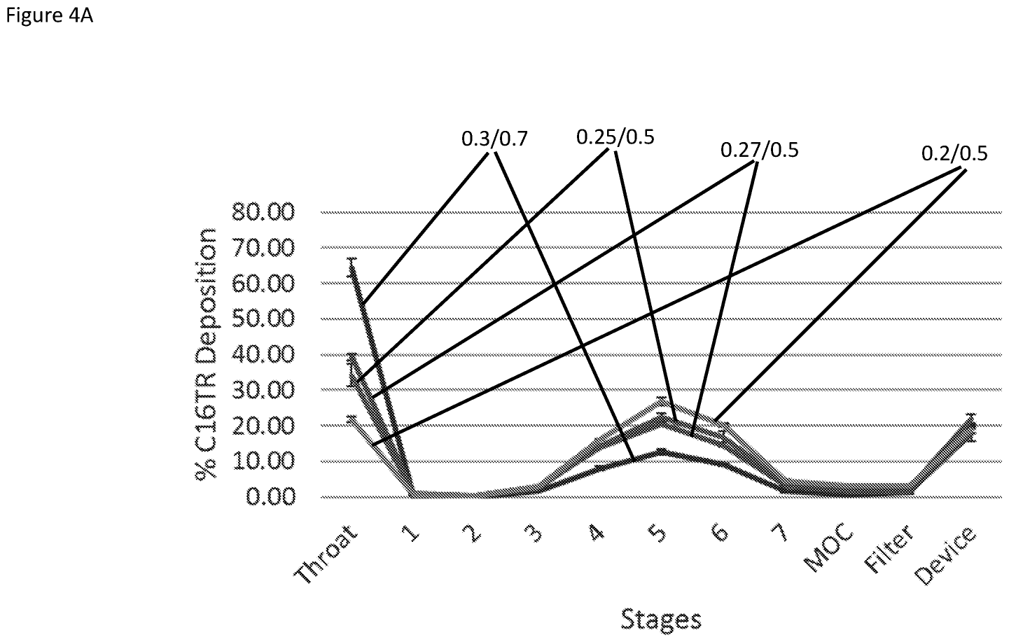

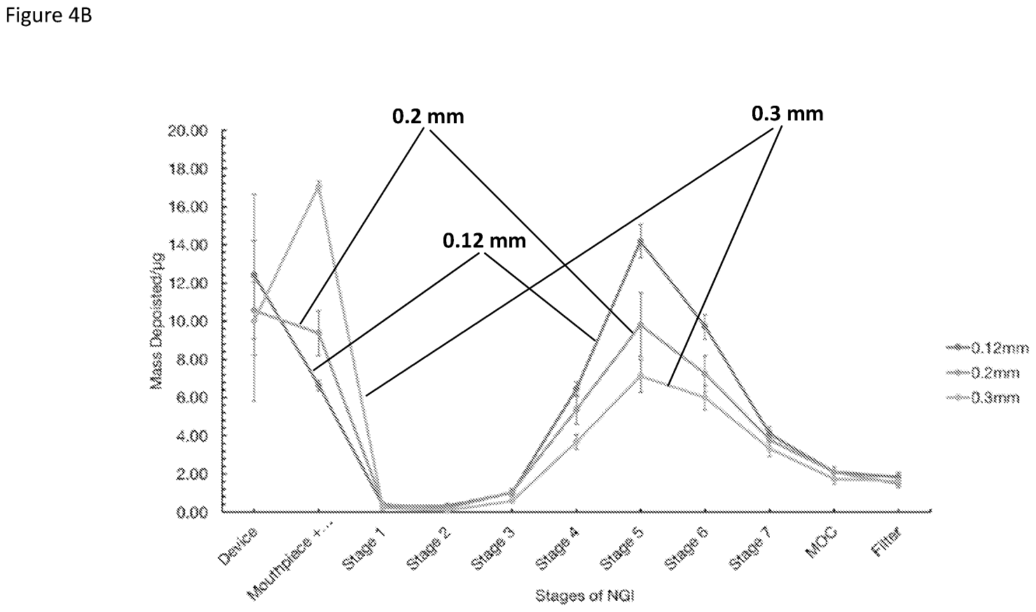

The results show that there is a general decrease in MMAD as orifice diameter decreases. There is a significant difference between 0.30/0.7 vs 0.20/0.5 as well as 0.27/0.5 vs 0.20/0.5 actuator sizes. As orifice diameter decreases, Fine Particle Fraction (FPF) increases significantly. As orifice diameter decreases, Fine Particle Dose (FPD) increases significantly. As orifice diameter decreases, throat deposition decreases creating a more favorable distribution. See FIGS. 4A and 4B. FIG. 4B shows that the 0.12 mm orifice diameter has the highest deposition in NGI stages 3-7. Overall, decreasing orifice diameter gives favorable and significant results, and an excellent delivery of C16TR can be achieved by using an appropriate actuator.

### Example 5—Aerosol Performance and “Dose Through Use” Measurements of the C16TR-MDI-W Formulation

### Methods

### 1. Aerodynamic Particle Size Distribution (APSD)-Next Generation Impactor (NGI)

The aerodynamic particle size distribution (APSD) of C16TR in the C16TR-MDI-W formulation was performed using the next generation impactor at a volumetric flow rate of 30 L/min under controlled temperature and humidity conditions of 23° C. and 50% RH using a Presspart MDI actuator. C16TR mass deposited on each NGI component was measured by HPLC/CAD to calculate delivered dose (DD), MMAD, throat deposition, FPF, and FPD.

### 2. “Dose Through Use” Study Protocol

Total six canisters were taken for the experiments. Out of the six, three canisters were positioned valve upright and three canisters were positioned valve down throughout the entire experiment period. All the canisters were prepared using a two-stage filling process in which a concentrated feedstock was added to empty cans, the cans were then crimped with a metering valve, and propellant was added to the cans. For this study Aptar DF 316/50 RCU CS20 ARGENT valves were used in conjunction with PressPart 19 mL plain aluminum canisters (Presspart, C0128-000). The study also used PressPart actuators (0.2 mm ID/0.5 mm JL) equipped with dose counters.

### 3. Dose Through Use Emitted Dose Assay (EDA)

The emitted dose assay (EDA) for the quantification of total mass delivered for the C16TR-MDI-W formulation was performed using dose uniformity sampling apparatus (DUSA). The sample collection unit was equipped with DUSA tube, 25 mm glass fiber filter (Pall Life Sciences, Part No. 61630), and a vacuum pump capable of pulling vacuum at 28.3 L/min volumetric flow rate or higher. The canister was actuated 2 times using Presspart actuator (0.3 mm Orifice Diameter/0.7 mm Jet Length), and the sample was collected by adding 20 mL of 75% IPA in H₂O inside the collection tube, vigorously shaking it to break the filter and finally filtered the sample using 1-mL syringe with 0.45 μm filter (Whatman, cat no. 10463050) unit. The quantitation of C16TR in the C16TR-MDI formulation deposited on each NGI component was accomplished via a calculation of internal seven-point linear log-log calibration curve of the log of the peak area of C16TR versus the log of the standard concentration over the nominal range of 0.4 μg/mL to 25 μg/mL using high performance liquid chromatography (HPLC) and Charged Aerosol Detector (CAD) on a C8 column.

4. “Dose through use”—Residual Material Analysis

Drug deposition in the inner wall of MDI canisters may happen because of adhesion or adsorption and may be affected by degradation of formulation components. The loss of drug in the inner wall may result in variability of the emitted dose. For drug deposition analysis, the canisters were actuated till dry (i.e. additional actuations did not emit any material) and the valves were cut using an InnovaSystems AC-2 automated can cutter. The remaining open-topped aluminum cans were dried for 2 hours at ambient condition. The cans were then visually observed to see any presence of deposited drug and taken a picture for record. Afterwards the whole canister was strongly rinsed (vortexed) with 3 mL of 75% IPA in H₂O and finally the sample was analyzed using HPLC.

### Results

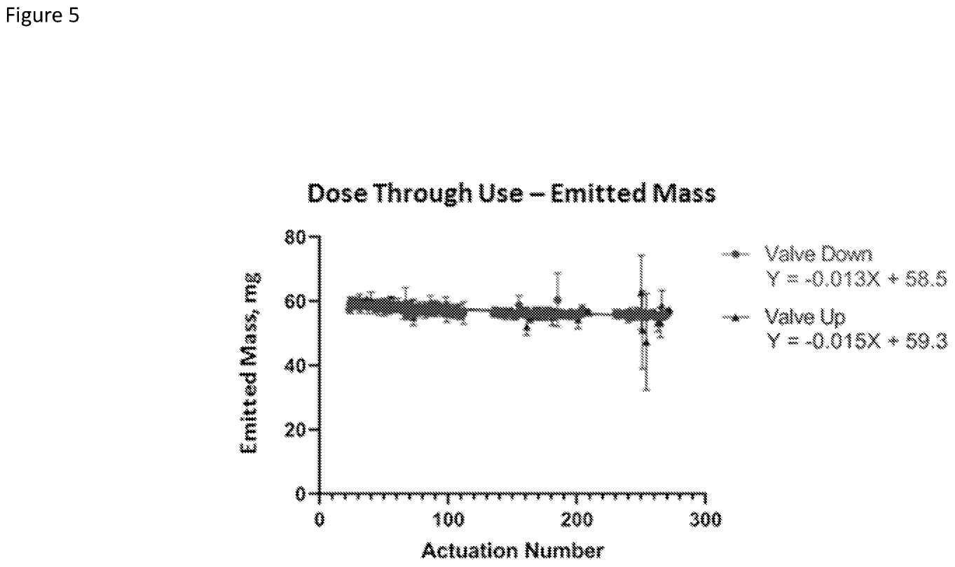

The “dose through use” study results for the C16TR-MDI-W formulation, as shown in FIGS. 5, 6A, 6B, 7A, and 7B and Tables 9 and 10, indicate that both the valve up and valve down canisters have produced reproducible results. In a summary, the valve up canisters were actuated 270 times and the average emitted mass was 57.33 mg±0.58 mg. At the same time, the valve down canisters were actuated 269 times and the average emitted mass was 57.00 mg±1.00 mg. A slight decrease in emitted mass was observed for all the canisters towards the ending of the experiments (Initial=58.67 mg±1.24 mg; End=55.39 mg ±0.75 mg). In accordance with traditional MDI testing guidelines, the emitted mass does not drift more than ±20% from the initial value. The above results indicate that the C16TR-MDI-W has an acceptable “dose through use” emitted mass and that the C16TR-MDI-W formulation is compatible with the current valve. The test also determined that the metering valve is capable in terms of measuring and delivering proper dose when paired with MDI formulations that use IPA as an alcohol co-solvent.

TABLE 9
Summary of Valve-Up Dose Through
Use Stability Studies of MDI-W

| | Beginning | Middle | End |
|---|---|---|---|
| DD, μg (n = 12) | 88.9 | 88.0 | 80.7 |
| MMAD, μm (n = 3) | 1.51 | 1.42 | 1.47 |
| % Throat Deposition (n = 3) | 21.5 | 16.1 | 17.5 |
| % FPF (<5 μm) (n = 3) | 75.0 | 81.2 | 78.2 |
| FPD, μg (n = 3) | 33.26 | 35.51 | 32.41 |

TABLE 10
Summary of Valve-Down Dose Through
Use Stability Studies of MDI-W

| | Beginning | Middle | End | p-Value |
|---|---|---|---|---|
| DD, μg (n = 12) | 90.2 | 88.0 | 86.4 | 0.030 |
| MMAD, μm (n = 3) | 1.49 | 1.53 | 1.47 | 0.185 |
| % Throat Deposition (n = 3) | 21.1 | 20.2 | 18.5 | 0.092 |
| % FPF (<5 μm) (n = 3) | 75.1 | 76.0 | 76.9 | 0.399 |
| FPD, μg (n = 3) | 34.65 | 32.80 | 32.78 | 0.347 |

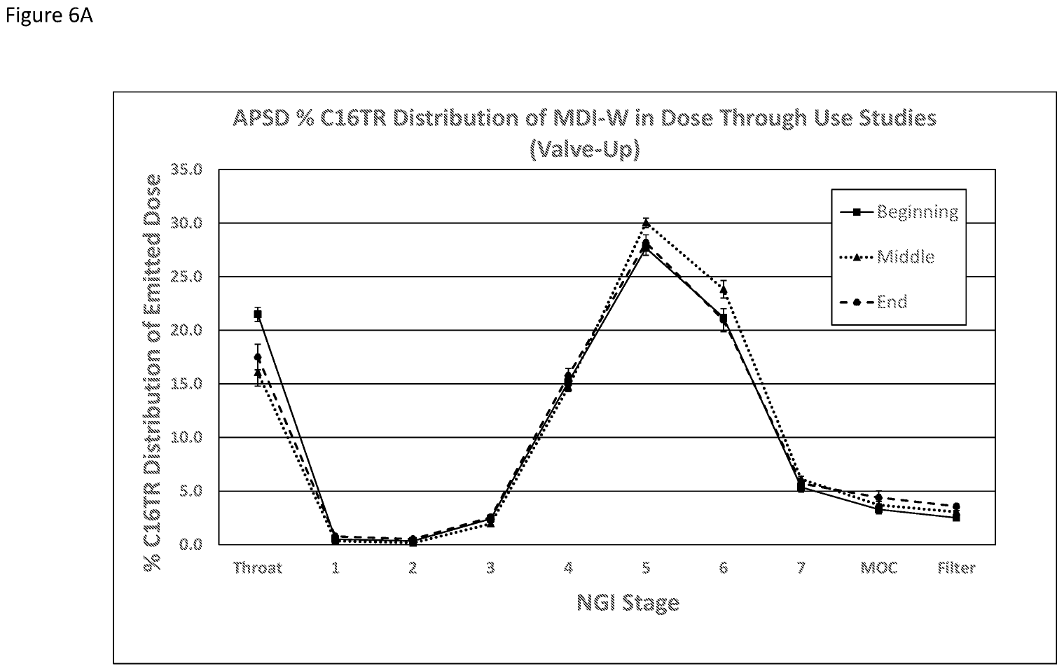

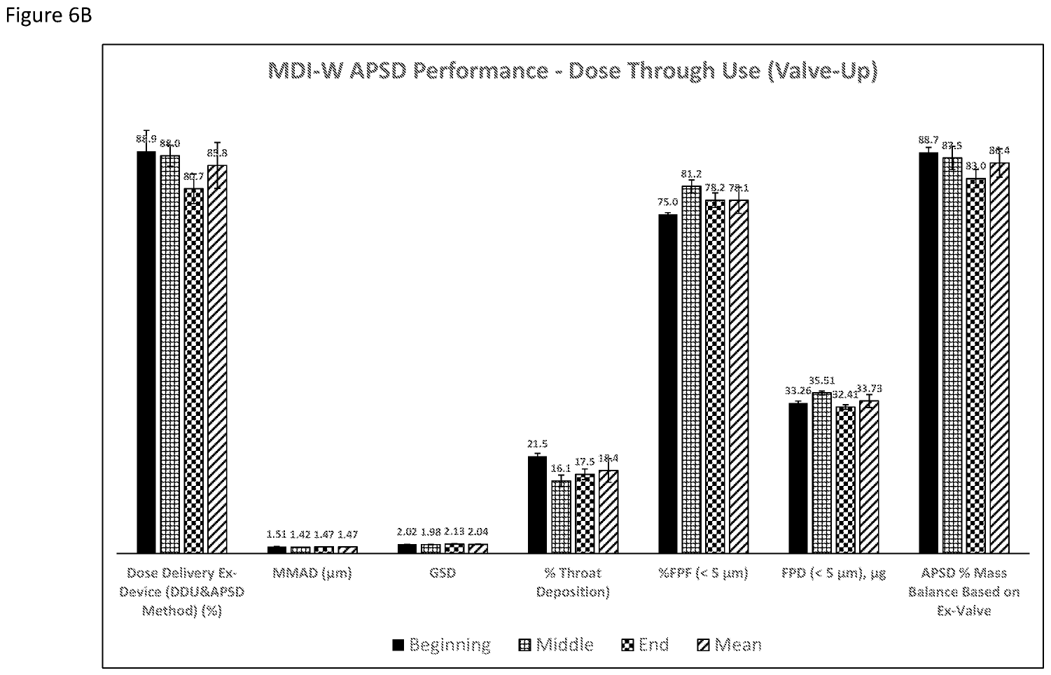

APSD results using NGI show that the aerosol performance of the C16TR-MDI-W formulation was comparable over time. For the valve up canisters, the MMAD is around 1.45 (±0.05) μm and the throat deposition is around 18.36 (±2.80) and fine particle fraction (FPF) is around 78.13 (±3.10). Although the APSD summary for three separate tests (Beginning, Middle and End) has a slight statistical difference, the overall results are satisfactory because of the proximity of the three data. The emitted dose calculations for “valve up” canisters are all >80% of the theoretical value based on formulation strength and actuation volume. See data in FIGS. 6A and 6B and Table 9.

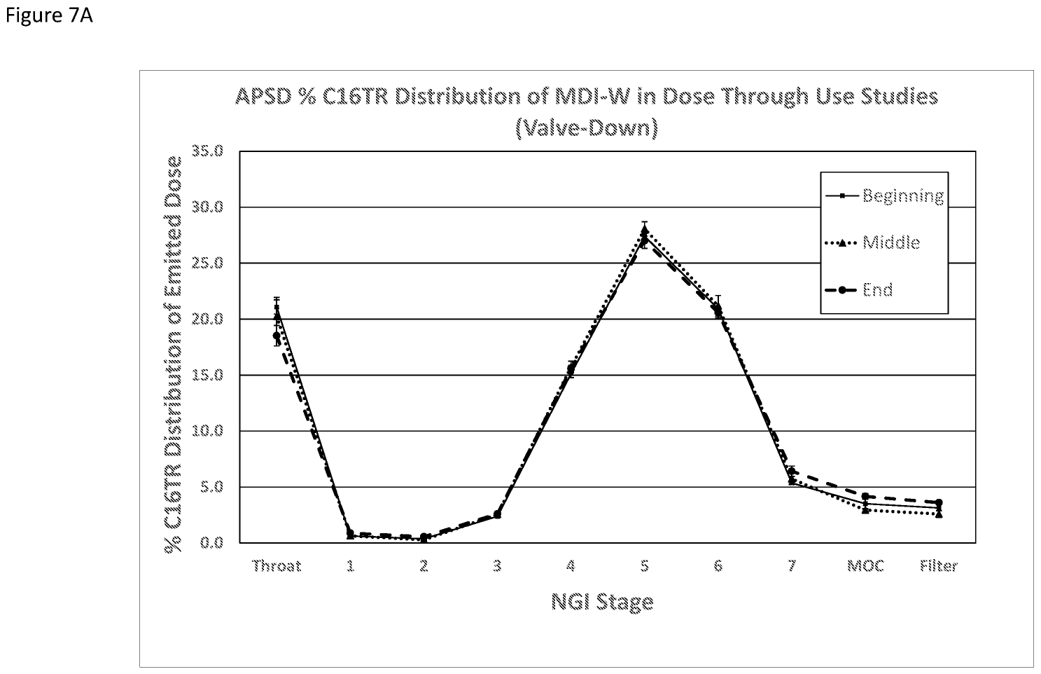

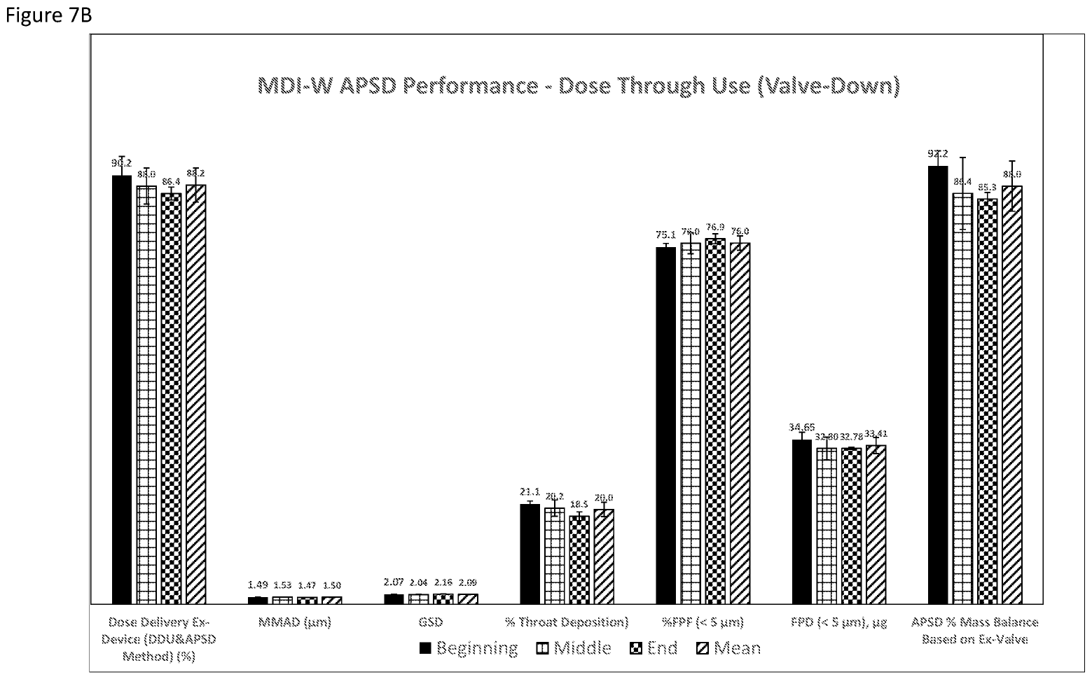

For the valve down canisters, the MMAD is around 1.49 (±0.03) μm and the throat deposition is around 19.93 (±1.32) and fine particle fraction (FPF) is around 76.0 (±0.9). The APSD summary for three separate tests (Beginning, Middle and End) has no statistical difference. Overall, a similar result was observed for the beginning, middle and end results. The emitted dose calculations for “valve up” canisters are all >85% of the theoretical value based on formulation strength and actuation volume. See data in FIGS. 7A and 7B and Table 10.

Residual material analysis showed that the canister wall was clean and there was no deposited drug found inside the wall. However, the canister was rinsed with 75% IPA in H₂O and analyzed in HPLC. The HPLC data suggests a presence of a tiny amount C16TR (17 μg+33.4) and DSPE-P2K (4.80 μg ±20.18), though that may be because the formulation was not fully emitted before being cut open.

The results of this example demonstrate that the C16TR-MDI-W formulation was a stable formulation in terms of emitted mass measurement, aerosol performance and drug deposition on the canister walls. In light of the MDI testing guideline that the emitted mass should not drift more than 20% from the initial value, the results from this example show that the C16TR-MDI-W formulation has an acceptable “Dose Through Use” emitted mass and the formulation is compatible with the current valve. The results also indicate that the metering valve was suitable in measuring and delivering proper dose of the MDI-W formulation that uses IPA as an alcohol co-solvent. Additionally, the NGI data reveals that the MMAD is around 1.45 (±0.05) μm and 1.49 (±0.03) μm for valve up and valve down canisters, respectively, with fine particle fraction (FPF) around 78.13% (±3.10) and 76.0% (±0.9) accordingly. At the same time, the emitted dose/delivered dose for both valve up and valve down canisters were around >80% of the Label claim. All these APSD outcomes were satisfactory. When drug deposition on canister wall was analyzed at the very end, a clear canister wall was observed, and no deposited drug was found, which shows that the formulation was stable inside the plain aluminum canisters.

### Example 6—Effect of DSPE-PEG2000 on Formulation Performance

To evaluate the impact of DSPE-PEG2000 degradation on drug performance, we prepared and studied an artificially degraded sample, formulation MDI-X, using both in vitro and in vivo tests (see Example 7 below). The results indicate that MDI-X performs comparably to MDI-W in each test suggesting that degradation of DSPE-PEG2000 is inconsequential.

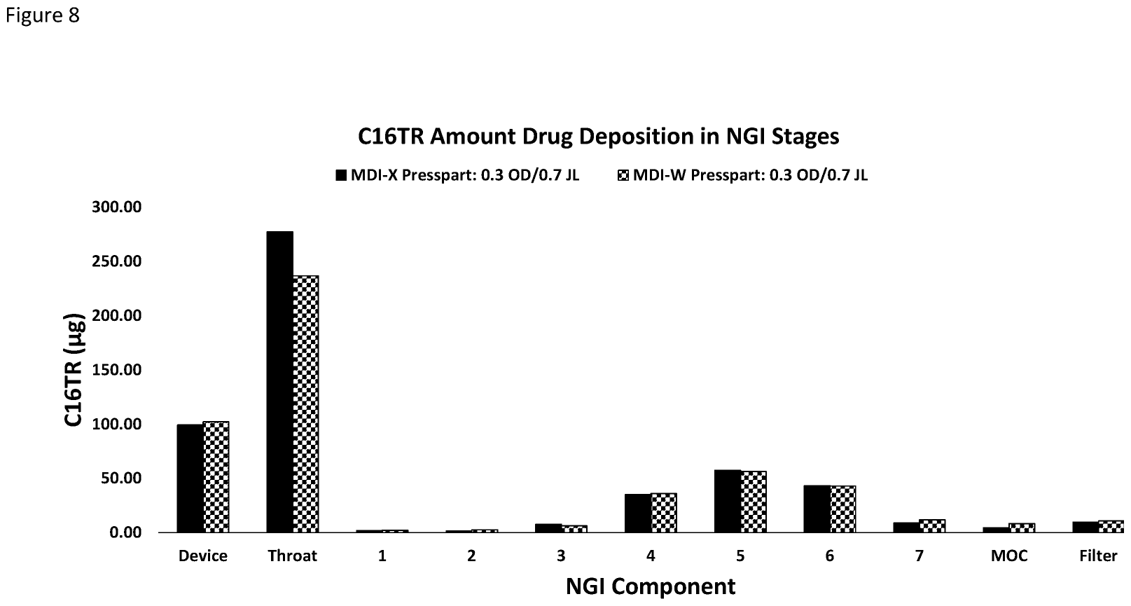

Comparison of APSD data between MDI-X and MDI-W under identical test conditions indicates comparable aerosol performance for the two formulations (Table 11 and FIG. 8). The MMAD, GSD, throat deposition, and FPF appear to be comparable between the two formulations. This data is consistent with both our solubility data and our in vivo performance data (shown in Example 7 below), suggesting that the two formulations are comparable. Collectively, all data with MDI-X suggests that degradation of DSPE-PEG2000 to MSPE-PEG2000 and stearic acid is acceptable in that it does not affect the performance of the formulation in vitro or in vivo.

TABLE 11
APSD comparison of MDI-X and MDI-W

| APSD Comparison of MDI-W and MDI-X | | | | | | | | | | | | |
|---|---|---|---|---|---|---|---|---|---|---|---|---|
| | | | | NGI | NGI Mass | | | | | | | |
| | Total | | | Total | Balance | | | | | | | |
| | Delivered | | | Recovery | Based on | | | Throat | | | | |
| | Mass | | | Based on | Total | | | Deposition | Calculated | | From Group Stages | |
| | C16TR | | C16TR | Total | Delivered | | | Based on | FPF | FPD | FPF | FPD |
| | (Ex- | # Of | Emitted | Delivered | Mass (Ex- | | | Emitted | (<5 | (<5 | (Stages | (Stages |
| Formulation | Valve) | Shots | Dose | Mass | Device) | MMAD | | Dose | μm) | μm) | 3 - F) | 3 - F) |
| # | μg | # | μg | % | % | μm | GSD | % | % | μg/shot | % | μg/shot |
| MDI-X | 591.819 | 10 | 447.400 | 92.4 | 75.6 | 1.59 | 2.13 | 62.0 | 35.5 | 15.905 | 37.2 | 16.663 |
| MDI-W | 550.996 | 10 | 414.077 | 93.7 | 75.2 | 1.51 | 2.23 | 57.2 | 39.9 | 16.533 | 41.7 | 17.250 |

### Example 7 —In Vivo Performance Studies of the C16TR-MDI-W and C16TR-MDI-X Formulations

In this example, the lung and plasma pharmacokinetics of the C16TR-MDI-W formulation at a low delivered dose (62.2 μg/kg body weight) and a high delivered dose (115 μg/kg body weight) and the C16TR-MDI-X formulation in rats were investigated and compared with the lung and plasma pharmacokinetics of nebulized INS1009. The pulmonary vasodilating effects of the C16TR-MDI-W and C16TR-MDI-X formulations in hypoxia-challenged telemetered rats were also evaluated.

### Methods

### 1. Lung and Plasma Pharmacokinetics Study

For the Lung and plasma pharmacokinetics study, nose only inhalation and blood and lung sampling in rats, pharmacokinetic analysis, and delivered dose calculations were performed as described in Example 1.

### 2. Pulmonary Vasodilating Efficacy Study

Pulmonary vasodilating efficacy of the C16TR-MI-W and C16TR-MDI-X formulations was determined in rats that were prepared with a telemetry probe implanted in the right ventricle to measure the inhibition of the increase in right ventricular pulse pressure (RVPP) that was induced by exposure to an inhaled hypoxic gas mixture.

Specifically, experiments were performed in male Sprague Dawley rats that were implanted with telemetry probes in the right ventricle and descending aorta to measure RVPP and mean systemic arterial blood pressure (mSAP). These cardiovascular parameters were measured while breathing normoxic air (21% O₂/balance N₂), following a 10-min exposure to hypoxic air (10% O₂/balance N₂) and returned to breathing normoxic air. The increase in RVPP due to the hypoxia challenge (A RVPP due to hypoxia) was measured before drug exposure and at 1, 6, 12 and 24 hours after exposure to inhaled test articles.

Nose-only inhalation studies in rats were performed using an inhalation tower modified for MDI delivery. Cohorts of 11 rats were used with a filter connected to the one remaining outlet port from which the aerosol concentration in the nose-only chamber was measured. Air was circulated through the chamber at a flow rate of 20 L/min. A vacuum pump was connected to the filter and set at a vacuum flow of either 2.0 or 3.0 L/min which began at 5 min after the start of the aerosolization of the drug and ended 1, 2, or 3 min later, i.e. filter sampling time of 1, 2, or 3 min depending on the formulation concentration and the number of canisters used. The filter samples were analyzed for C16TR measured by HPLC with a CAD.

### Results

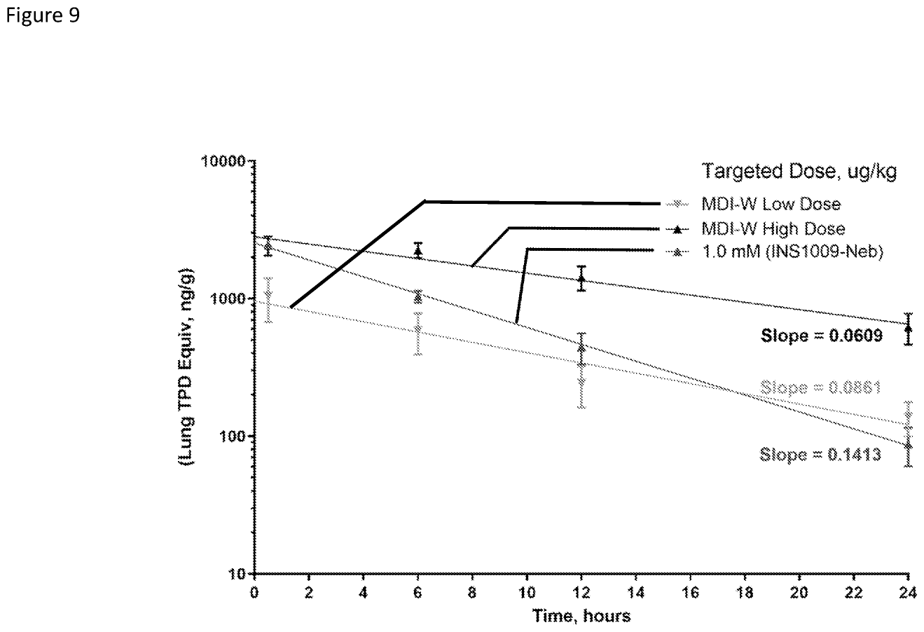

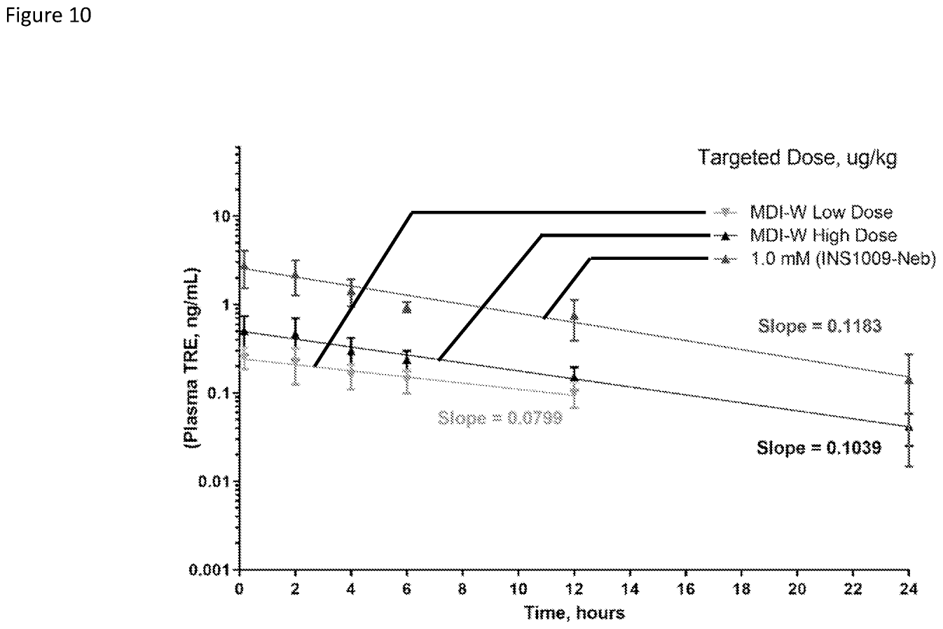

The pharmacokinetic results of the C16TR-MDI-W formulation are shown in FIGS. 9 and 10. The results of FIG. 9 indicate that the C16TR lung clearance with the C16TR-MDI-W formulation is slower than that with nebulized INS1009 as indicated by the slope. The results of FIG. 10 indicate that the plasma TRE levels with the C16TR-MDI-W formulation are acceptable in that they remain above 0.1 ng/mL for 12-24 hours and are consistently decreasing.

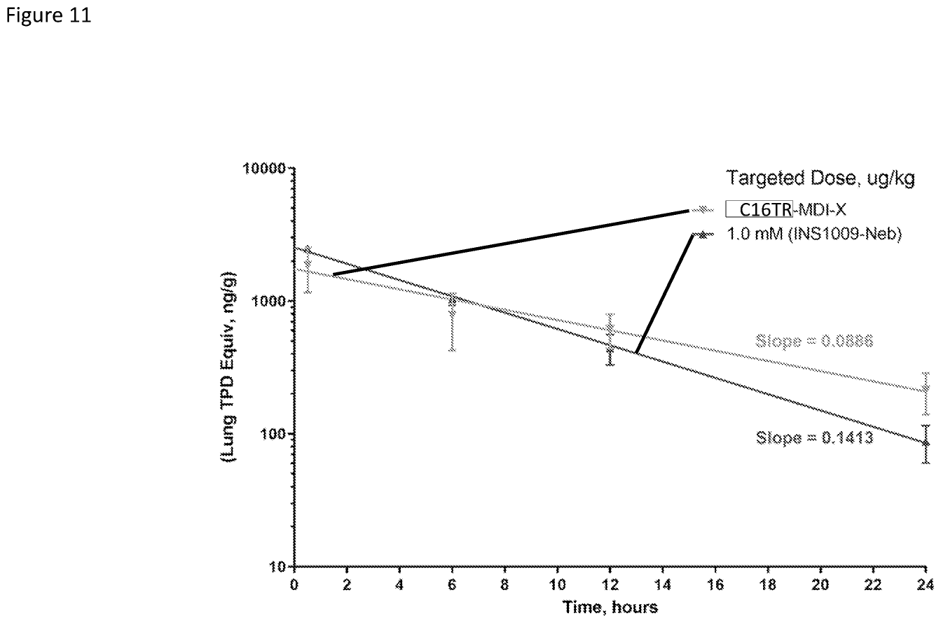

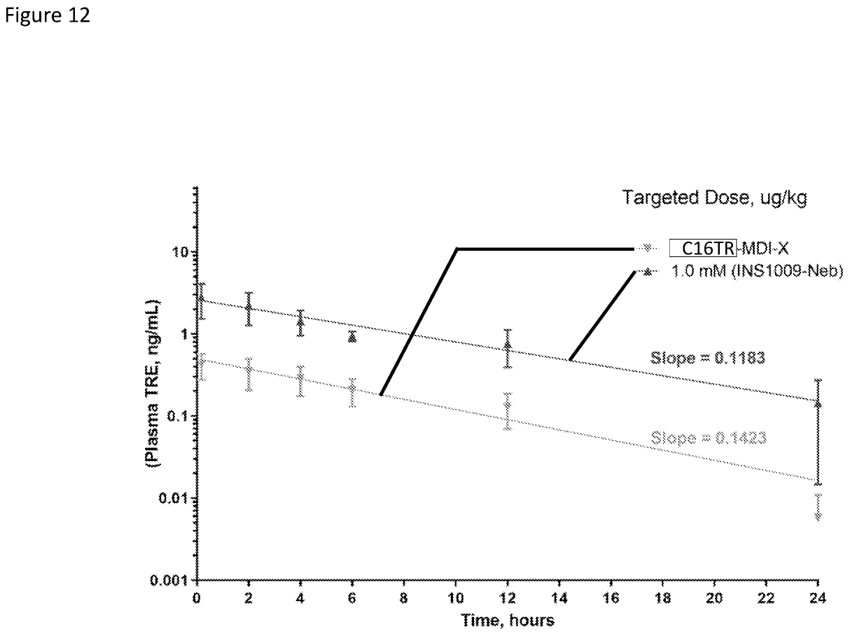

The pharmacokinetic results of the C16TR-MDI-X formulation are shown in FIGS. 11 and 12. The results of FIG. 11 indicate that the C16TR lung clearance with the C16TR-MDI-X formulation is slower than that with nebulized INS1009 but faster than that of the C16TR-MDI-W formulation as indicated by the slope. The results of FIG. 12 indicate that the plasma TRE levels with the C16TR-MDI-X formulation are excellent as compared to those of nebulized INS1009 in that they remain above 0.1 ng/mL for 12-24 hours and are consistently decreasing.

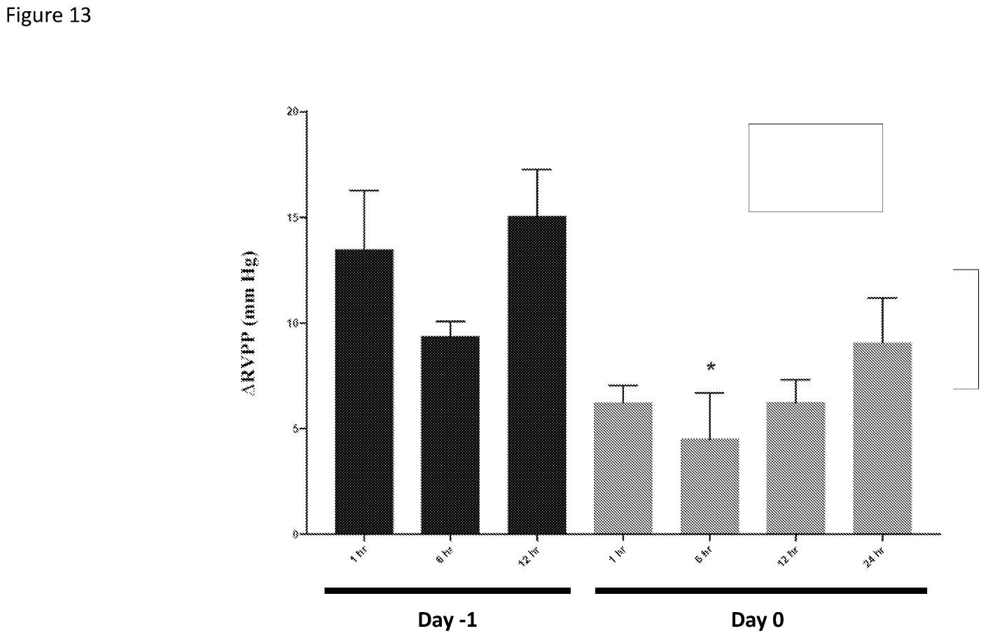

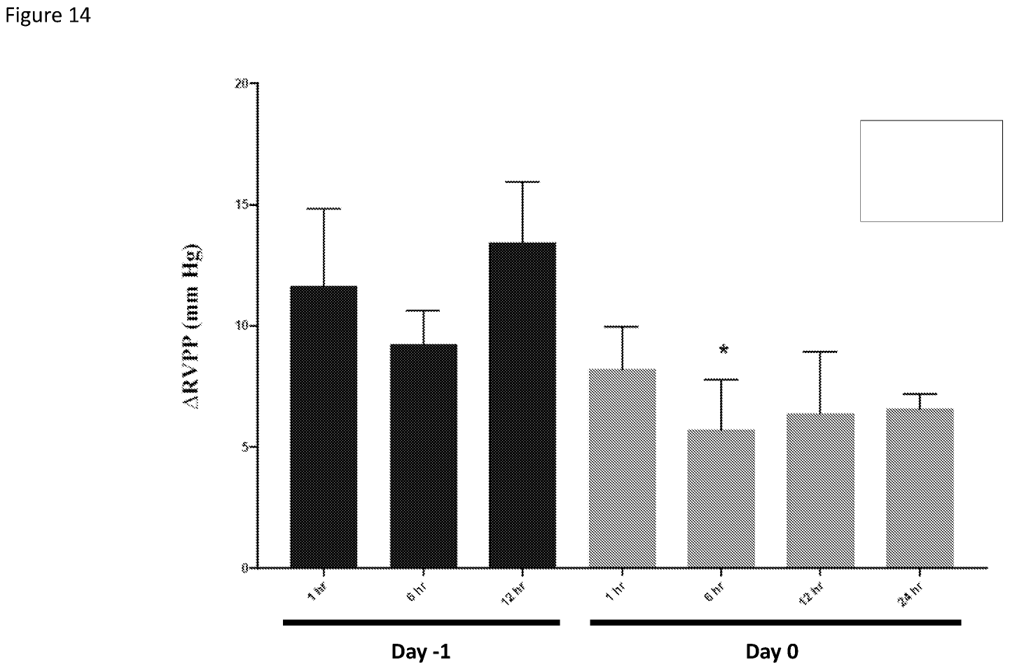

Exposure of rats to inhaled hypoxia increased RVPP by approximately 10-20 mmHg over the normoxia values, depending on the individual study. At the end of the hypoxia challenge, the RVPP immediately decreased within a few minutes and returned to the pre-hypoxia values within 10 minutes. The drug effects were determined by comparing the ΔRVPP due to hypoxia at various times up to 24 hours after drug exposure to the combined value from 2-3 determinations obtained before drug exposure. Exposure to C16TR-MDI-W at a delivered dose of 115 μg/kg reduced the ΔRVPP due to hypoxia with statistically significant (p<0.05) inhibition observed at 6 hours (FIG. 13), though within 24 hours the RVPP was trending back to the baseline value observed before drug exposures. Similar results were obtained with exposure to C16TR-MDI-X where an inhaled dose of 111 μg/kg reduced the ΔRVPP due to hypoxia with statistically significant (p<0.05) inhibition observed at 6 hours (FIG. 14). Interestingly, in this instance the RVPP was not trending back to the baseline value observed before drug exposures. The delivered doses of C16TR-MDI-W and C16TR-MDI-X were based on the concentration of drug inhaled, the duration of exposure, body weight, respiratory minute volume and a deposition fraction of 1.0. See Alexander et al., Inhal. Toxicol. 20:1179-1189 (2008), incorporated herein by reference in its entirety.

Taken together, the result in this example indicate that both C16TR-MDI-W and C16TR-MDI-X are efficacious in lowering the pulmonary arterial pressure in hypoxia-challenged rats following inhalation of the formulation via an MDI, and that degradation of DSPE-PEG2000 does not impact the in vivo performance.

### Example 8—Particle Size Upon Dissolution Study of the C16TR-MDI-W and C16TR-MDI-X Formulations

In this example, we investigated whether the C16TR-MDI-W and C16TR-MDI-X formulations formed nanoparticles upon dissolution in aqueous media. To this end, samples of MDI-W and MDI-X were prepared and the nanoparticle size distribution was determined by Mobius. Specifically, the MDI formulation was actuated into an aqueous environment and then diluted for particle size analysis using a Mobius dynamic light scattering instrument.

As shown in Table 12, the C16TR-MDI-W and C16TR-MDI-X formulations formed nearly identical sized nanoparticles upon dissolution.

TABLE 12
Particle Size Upon Dissolution Measurements
for MDI-W and MDI-X

| | PSD evaluation of MDI-W & MDI-X | | | | | |
|---|---|---|---|---|---|---|
| | | | | | | Normalized |
| | | Diameter | | | % | Intensity |
| Formulation | Run | (nm) | Average | STD | PD | (kCnt/s) |
| MDI-W | 1 | 153.6 | 150 | 3.22 | 23.9 | 18343 |
| | 2 | 149 | | | 21.6 | 17221 |
| | 3 | 147.4 | | | 23.7 | 19378 |
| MDI-X | 1 | 157.4 | 158.4 | 3.08 | 22.1 | 24327 |
| | 2 | 161.9 | | | 22 | 23947 |
| | 3 | 156 | | | 22.5 | 23817 |

### Example 9—Assessment of C16TR-MDI Formulations on Cough and Ventilation in Guinea Pigs

In this example, C16TR-MDI formulations were evaluated for effects to produce cough, change in ventilation and change in Penh, a dimensionless index of altered breathing pattern typically seen during bronchoconstriction, in conscious guinea pigs.

### Methods

Experiments were performed in male Hartley guinea pigs. After a 3-day period of acclimation, the guinea pigs were placed in a whole body plethysmograph for the measurement of ventilation (tidal volume, respiratory rate and minute volume), Penh and cough using established techniques (see Corboz et al., J Pharmacol Exp Ther 363:1-10 (2017); Chong BTY et al., J. Pharmacol. Toxicol. Methods 39,163-168 (1998); Lomask M., Exp. and Toxicol. Pathol. 57,13-20 (2006), each of which is incorporated herein by reference in its entirety). Cough was measured from plethysmograph recordings showing a large inspiration followed by a large expiration and confirmed by manual observations, video recordings and cough sounds. The ventilation, Penh and cough data were measured during a 15 min baseline period while breathing humidified air that was circulated through the plethysmograph. The test articles, which included MDI-L, MDI-W, MDI-TRE, and their respective vehicles controls, were then delivered by metered dose inhalers for 15 min followed by a 120 min observation period after the aerosol compounds were given. Ventilation, Penh and cough were measured both before, during and after exposure to the test articles.

The delivery system was fed with a flow rate of 2 L/min through the inlet of the plethysmograph. A vacuum bias flow of 1.6 L/min was maintained during the whole experiment period, with an additional 0.5 L/min vacuum connected through a filter for sample inhalation analysis. The filter sampling was maintained for the full duration of the study i.e. 135 min, but a 10— or 15-min exposure time was used to calculate the aerosol concentration of the drug in the nose-only chamber (see Alexander D J et al., Inhal. Tox. 20:1179-1189 (2008), incorporated herein by reference in its entirety). The filter samples were analyzed for the C16TR concentration, with the data used for delivered dose calculations. At the end of the study the guinea pigs were euthanized and blood (plasma), lungs, trachea, larynx and carina+bronchi were collected to measure the C16TR and TRE concentrations in these samples; tissue samples were not collected from guinea pigs treated with MDI-TRE or vehicle controls.

### Results

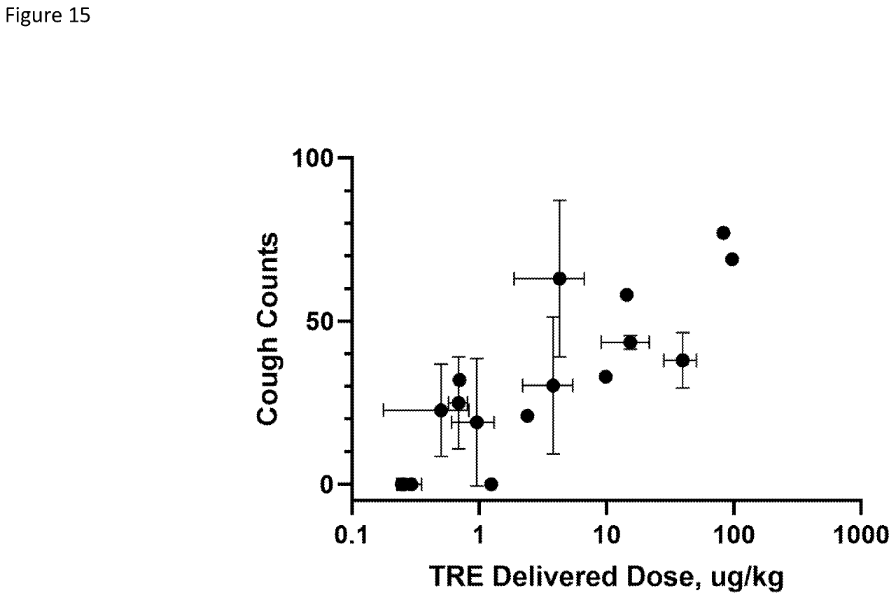

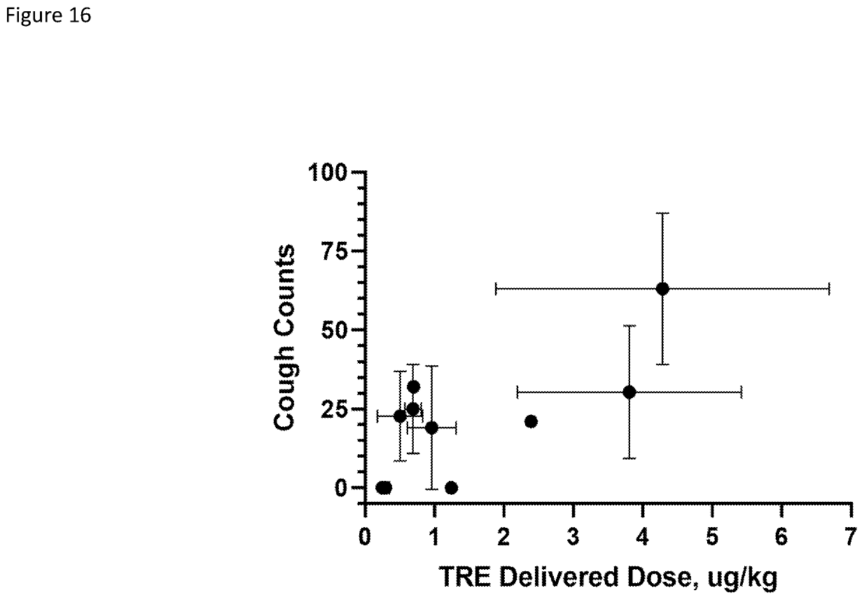

Exposure of MDI-TRE was tolerated at extremely low doses but caused significant cough at higher doses. Plots of cough counts vs. TRE delivered dose are shown in FIGS. 15 and 16. To identify the highest delivered dose that did not produce a cough response, i.e., the “no-cough” delivered dose, we titrated the dose down until we did not observe cough. Based on our data the highest delivered dose that does not cause cough in guinea pigs was 0.293 μg/kg. This data confirms that guinea pig cough responses are extremely sensitive to exposure of treprostinil acid delivered by MDI. Error bars represent standard deviation; each point represents data collected from a single day's worth of experiments.

Exposure of vehicle controls containing either isopropyl alcohol (IPA) or IPA and excipients (DSPE-PEG2000 and PEG400) were well tolerated and did not produce any cough response in guinea pigs.

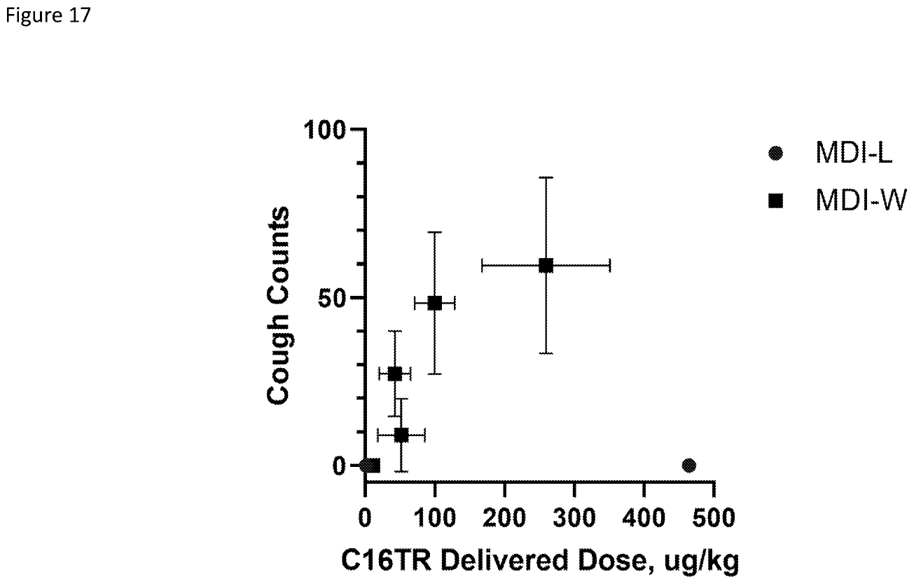

Exposure of MDI-L and MDI-W were well tolerated and did not result in mortality. A plot of cough counts vs. C16TR delivered dose is shown in FIG. 17. In terms of avoiding a cough response MDI-L appears to be the better formulation in that no cough was observed even at high dose levels. Note that each result for MDI-L was generated from a single guinea pig. Error bars represent standard deviation; each point represents data collected from a single day's worth of experiments. The C16TR delivered dose was calculated based on the filter data.

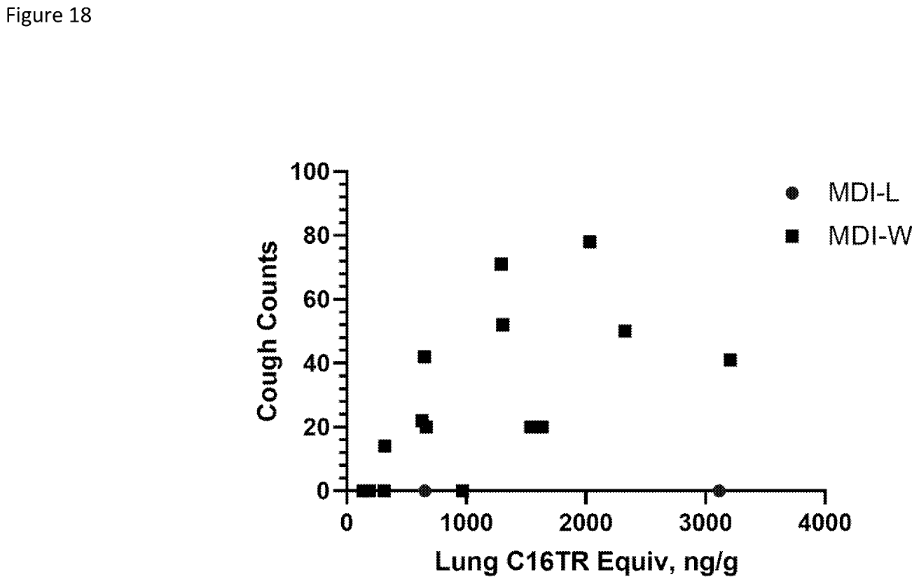

Another way to look at the data is to plot individual data points and change the x-axis to be the actual Lung C16TR equivalent levels measured during bioanalysis. With this analysis we get a different perspective on the data (FIG. 18). There is some discrepancy between the filter data and the amount of C16TR equivalents measured in the lungs 2 hours after dosing.

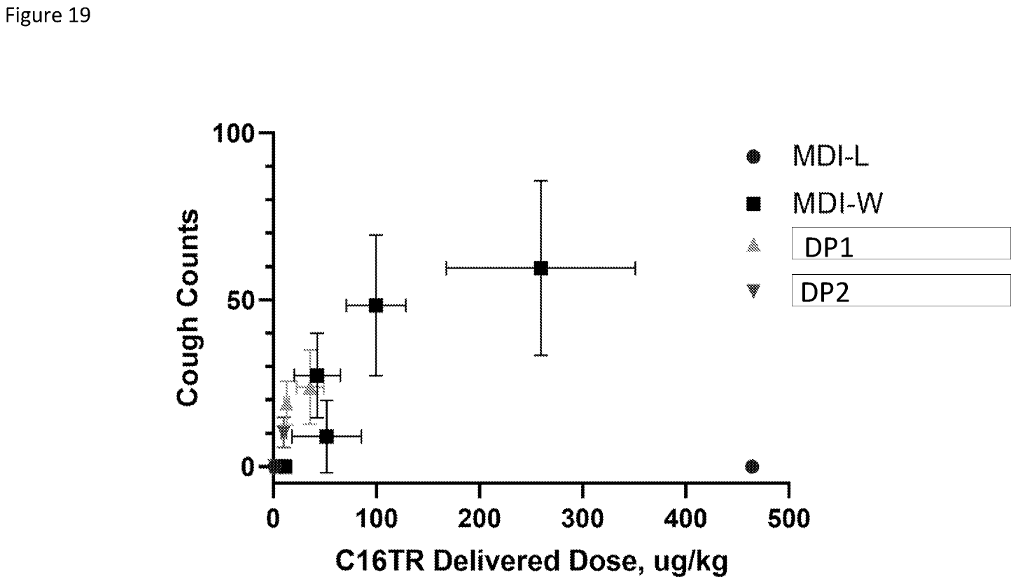

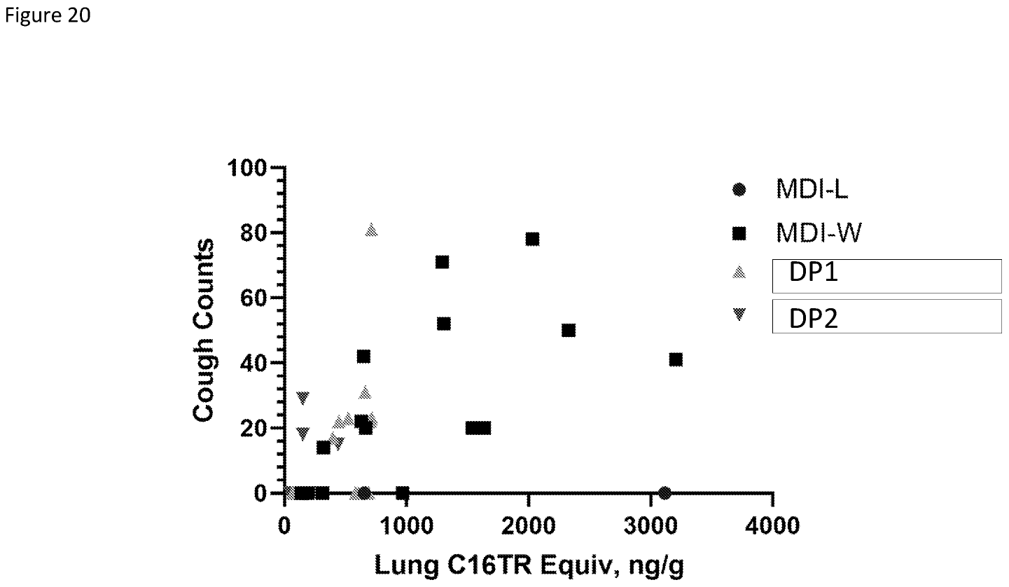

Comparison of cough response to MI-L, MI-W, and two dry powder formulations (C16TR-DP1 and C16TR-DP2) of C16TR yield comparable results (FIG. 19). C16TR-DP1 includes C16TR, DSPE-PEG2000, mannitol, and leucine in a weight ratio of 1.5:0.75:70:30. C16TR-DP2 includes C16TR, DSPE-PEG2000, trehalose, and leucine in a weight ratio of 1.5:0.75:70:30. There is a linear correlation between the C16TR delivered dose, and the average number of coughs observed. The results may indicate that the “no-cough” delivered dose of MDI-W may be higher than that of C16TR-DP1 and C16TR-DP2 formulations. Similar to the analysis presented above, we can compare the different formulations and routes of administration by plotting cough counts vs. lung C16TR equivalent levels measured during bioanalysis (FIG. 20). With this analysis we get a slightly different perspective on the data, but come to the same conclusion.

In summary, cough was observed with both MDI-TRE and MI-W but was not observed for MDI-L or vehicle controls. The animals appear to be extremely sensitive to treprostinil acid delivered by MDI with large amounts of coughs being observed even at relatively low delivered doses. The “no-cough” delivered dose for MDI-W (11.5 μg/kg) was higher than that observed for C16TR-DP1 (4.7 μg/kg) and C16TR-DP2 (2.3 μg/kg), suggesting that guinea pigs may tolerate C16TR delivered by an MDI better than a dry powder inhaler. However, comparing measured lung C16TReq levels the no-cough level for MDI-W was 313 ng/g, similar to the levels observed previously for C16TR dry powder formulations.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A pharmaceutical formulation comprising:
(a) a compound of Formula (Ia): 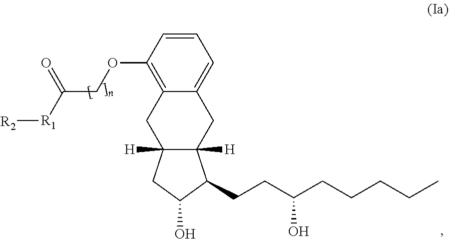
or an enantiomer, diastereomer, or pharmaceutically acceptable salt thereof, wherein R₁ is O; R₂ is dodecyl, tridecyl, tetradecyl, pentadecyl, hexadecyl, heptadecyl or octadecyl; and n is an integer from 0 to 5;
(b) polyoxyethylene (20) cetylether or at least one polyethylene glycol-lipid (PEGylated lipid);
(c) a surfactant selected from polyethylene glycol (PEG), propylene glycol, or a combination thereof,
(d) at least one hydrofluoroalkane propellant, and
(e) at least one alcohol cosolvent.

2. The pharmaceutical formulation of claim 1, wherein (a) is a compound of Formula (Ia) or a pharmaceutically acceptable salt thereof.

3. The pharmaceutical formulation of claim 1 or 2, wherein n is 0 or 1.

4. The pharmaceutical formulation of claim 1 or 2, wherein n is 0.

5. The pharmaceutical formulation of claim 1 or 2, wherein n is 1.

6. A pharmaceutical formulation comprising:
(a) a compound of Formula (Ib): 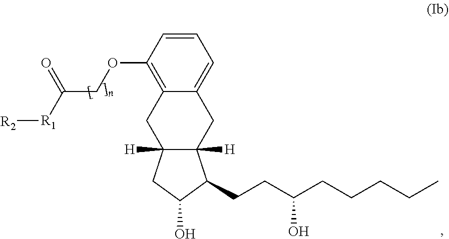
or an enantiomer, diastereomer, or pharmaceutically acceptable salt thereof, wherein n is O, R₁ is NH or O and R₂ is a linear C₅-C₁₈ alkyl,
(b) polyoxyethylene (20) cetylether or at least one polyethylene glycol-lipid (PEGylated lipid),
(c) a surfactant selected from PEG, propylene glycol, or a combination thereof,
(d) at least one hydrofluoroalkane propellant, and
(e) at least one alcohol cosolvent.

7. The pharmaceutical formulation of claim 6, wherein (a) is a compound of Formula (Ib) or a pharmaceutically acceptable salt thereof.

8. The pharmaceutical formulation of claim 6 or 7, wherein R₁ is NH.

9. The pharmaceutical formulation of claim 6 or 7, wherein R₁ is O.

10. The pharmaceutical formulation of any one of claims 6-9, wherein R₂ is linear heptyl, linear octyl, linear nonyl, linear decyl, linear undecyl, linear dodecyl, linear tridecyl, linear tetradecyl, linear pentadecyl, linear hexadecyl, linear heptadecyl or linear octadectyl.

11. The pharmaceutical formulation of any one of claims 1-10, wherein the compound of Formula (Ia) or (Ib), or an enantiomer, diastereomer, or pharmaceutically acceptable salt thereof, is present at a concentration of from about 0.5 to about 3 mg/mL.

12. The pharmaceutical formulation of claim 11, wherein the compound of Formula (Ia) or (Ib), or pharmaceutically acceptable salt thereof is present at a concentration of from about 0.5 to about 3 mg/mL.

13. The pharmaceutical formulation of any one of claims 1-12, wherein the polyoxyethylene (20) cetylether is present at a concentration of from about 0.25 to about 0.75 mg/mL.

14. The pharmaceutical formulation of any one of claims 1-13, wherein the polyoxyethylene (20) cetylether is present at a concentration of about 0.5 mg/mL.

15. The pharmaceutical formulation of any one of claims 1-14, wherein the at least one PEGylated lipid is one PEGylated lipid.

16. The pharmaceutical formulation of claim 15, wherein the at least one PEGylated lipid is one PEGylated lipid selected from the group consisting of DSPE (distearoylphosphatidylethanolamine)-PEG2000, DSG (disteraroylglycerol)-PEG2000, and DPG (diphosphatidylglycerol)-PEG2000.

17. The pharmaceutical formulation of any one of claims 1-14, wherein the at least one PEGylated lipid consists of a double or triple combination of DSPE-PEG2000, DSG-PEG2000, and DPG-PEG2000.

18. The pharmaceutical formulation of any one of claims 1-17, wherein the at least one PEGylated lipid is present at a concentration of from about 0.2 to about 3 mg/mL.

19. The pharmaceutical formulation of any one of claims 1-18, wherein the surfactant is one surfactant selected from the group consisting of PEG400, PEG1000, and propylene glycol.

20. The pharmaceutical formulation of any one of claims 1-18, wherein the surfactant consists of a double or triple combination of PEG400, PEG1000, and propylene glycol.

21. The pharmaceutical formulation of any one of claims 1-20, wherein the surfactant is present at a concentration of from about 0.75 to about 6 mg/mL.

22. The pharmaceutical formulation of any one of claims 1-21, wherein the at least one hydrofluoroalkane propellant is one hydrofluoroalkane propellant.

23. The pharmaceutical formulation of claim 22, wherein the at least one hydrofluoroalkane propellant is one hydrofluoroalkane propellant selected from the group consisting of 1,1,1,2-tetrafluoroethane (HFA134a), 1,1,1,2,3,3,3-heptafluoro-n-propane (HFA227ea), and 1,1-difluoroethane (FA152a).

24. The pharmaceutical formulation of any one of claims 1-21, wherein the at least one hydrofluoroalkane propellant consists of a double or triple combination of HFA134a, HFA227ea, and HFA152a.

25. The pharmaceutical formulation of any one of claims 1-24, wherein the at least one alcohol cosolvent is one alcohol cosolvent.

26. The pharmaceutical formulation of claim 25, wherein the at least one alcohol cosolvent is one alcohol cosolvent selected from the group consisting of ethanol and isopropyl alcohol.

27. The pharmaceutical formulation of any one of claims 1-24, wherein the at least one alcohol cosolvent is a combination of ethanol and isopropyl alcohol.

28. The pharmaceutical formulation of any one of claims 1-27, wherein the at least one alcohol cosolvent is present at a concentration of from about 3% to about 10% (w/w) based on the total weight of the pharmaceutical formulation.

29. A pharmaceutical formulation comprising:
(a) a compound of Formula (II): 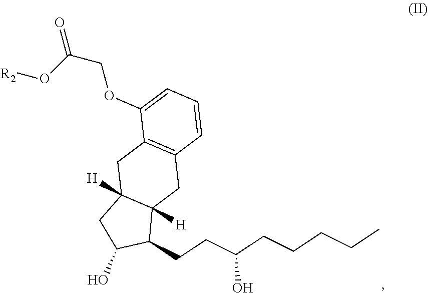
or an enantiomer, diastereomer, or pharmaceutically acceptable salt thereof at a concentration of from about 0.5 to about 3 mg/mL, wherein R₂ is dodecyl, tridecyl, tetradecyl, pentadecyl, hexadecyl, heptadecyl, or octadecyl,
(b) polyoxyethylene (20) cetylether at a concentration of from about 0.25 to about 0.75 mg/mL, or at least one PEGylated lipid selected from the group consisting of DSPE-PEG2000, DSG-PEG2000, and DPG-PEG2000 at a concentration of from about 0.2 to about 3 mg/mL,
(c) a surfactant selected from PEG400, PEG1000, propylene glycol, or a combination thereof at a concentration of from about 0.75 to about 6 mg/mL,
(d) at least one hydrofluoroalkane propellant selected from the group consisting of HFA134a, HFA227ea, and HFA152a, and
(e) at least one alcohol cosolvent selected from the group consisting of ethanol and isopropyl alcohol at a concentration of from about 3% to about 10% (w/w) based on the total weight of the pharmaceutical formulation.

30. The pharmaceutical formulation of claim 29, wherein (a) is a compound of Formula (II) or a pharmaceutically acceptable salt thereof.

31. The pharmaceutical formulation of claim 29 or 30, wherein the polyoxyethylene (20) cetylether is present at a concentration of about 0.5 mg/mL.

32. A pharmaceutical formulation consisting of
(a) a compound of Formula (II): 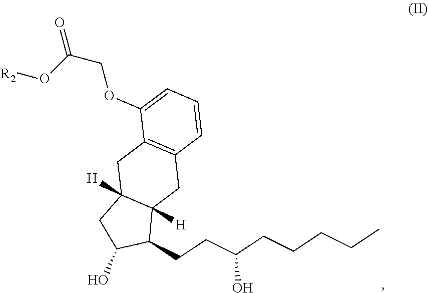
or an enantiomer, diastereomer, or pharmaceutically acceptable salt thereof at a concentration of from about 0.5 to about 3 mg/mL, wherein R₂ is dodecyl, tridecyl, tetradecyl, pentadecyl, hexadecyl, heptadecyl, or octadecyl,
(b) polyoxyethylene (20) cetylether at a concentration of from about 0.25 to about 0.75 mg/mL, or at least one PEGylated lipid selected from the group consisting of DSPE-PEG2000, DSG-PEG2000, and DPG-PEG2000 at a concentration of from about 0.2 to about 3 mg/mL,
(c) a surfactant selected from PEG400, PEG1000, propylene glycol, or a combination thereof at a concentration of from about 0.75 to about 6 mg/mL,
(d) at least one hydrofluoroalkane propellant selected from the group consisting of HFA134a, HFA227ea, and HFA152a, and
(e) at least one alcohol cosolvent selected from the group consisting of ethanol and isopropyl alcohol at a concentration of from about 3% to about 10% (w/w) based on the total weight of the pharmaceutical formulation.

33. The pharmaceutical formulation of claim 32, wherein (a) is a compound of Formula (II) or a pharmaceutically acceptable salt thereof.

34. The pharmaceutical formulation of claim 32 or 33, wherein the polyoxyethylene (20) cetylether is present at a concentration of about 0.5 mg/mL.

35. The pharmaceutical formulation of any one of claims 29-34, wherein the at least one PEGylated lipid is one PEGylated lipid selected from the group consisting of DSPE-PEG2000, DSG-PEG2000, and DPG-PEG2000.

36. The pharmaceutical formulation of any one of claims 29-34, wherein the at least one PEGylated lipid consists of a double or triple combination of DSPE-PEG2000, DSG-PEG2000, and DPG-PEG2000.

37. The pharmaceutical formulation of any one of claims 29-36, wherein the surfactant is one surfactant selected from the group consisting of PEG400, PEG1000, and propylene glycol.

38. The pharmaceutical formulation of any one of claims 29-36, wherein the surfactant consists of a double or triple combination of PEG400, PEG1000, and propylene glycol.

39. The pharmaceutical formulation of any one of claims 29-38, wherein the at least one hydrofluoroalkane propellant is one hydrofluoroalkane propellant selected from the group consisting of HFA134a, HFA227ea, and HFA152a.

40. The pharmaceutical formulation of any one of claims 29-38, wherein the at least one hydrofluoroalkane propellant consists of a double or triple combination of HFA134a, HFA227ea, and HFA152a.

41. The pharmaceutical formulation of any one of claims 29-40, wherein the at least one alcohol cosolvent is one alcohol cosolvent selected from the group consisting of ethanol and isopropyl alcohol.

42. The pharmaceutical formulation of any one of claims 29-40, wherein the at least one alcohol cosolvent is a combination of ethanol and isopropyl alcohol.

43. The pharmaceutical formulation of any one of claims 1-42, wherein the compound of Formula (Ia), (Ib), or (II), or an enantiomer, diastereomer, or pharmaceutically acceptable salt thereof is present at from about 0.5 to about 1 mg/mL in the pharmaceutical formulation.

44. The pharmaceutical formulation of claim 43, wherein the compound of Formula (Ia), (Ib), or (II), or pharmaceutically acceptable salt thereof is present at from about 0.5 to about 1 mg/mL in the pharmaceutical formulation.

45. The pharmaceutical formulation of claim 43, wherein the compound of Formula (Ia), (Ib), or (II), or an enantiomer, diastereomer, or pharmaceutically acceptable salt thereof is present at about 1 mg/mL in the pharmaceutical formulation.

46. The pharmaceutical formulation of claim 45, wherein the compound of Formula (Ia), (Ib), or (II), or pharmaceutically acceptable salt thereof is present at about 1 mg/mL in the pharmaceutical formulation.

47. The pharmaceutical formulation of any one of claims 1-16, 18-35, and 37-46, wherein the at least one PEGylated lipid is DSPE-PEG2000 present at from about 0.2 to about 3 mg/mL in the pharmaceutical formulation.

48. The pharmaceutical formulation of claim 47, wherein the DSPE-PEG2000 is present at from about 0.25 to about 0.75 mg/mL in the pharmaceutical formulation.

49. The pharmaceutical formulation of claim 47 or 48, wherein the DSPE-PEG2000 is present at about 0.5 mg/mL in the pharmaceutical formulation.

50. The pharmaceutical formulation of any one of claims 1-16, 18-35, and 37-46, wherein the at least one PEGylated lipid is DSG-PEG2000 present at from about 0.2 to about 0.5 mg/mL in the pharmaceutical formulation.

51. The pharmaceutical formulation of claim 50, wherein the DSG-PEG2000 is present at about 0.25 mg/mL in the pharmaceutical formulation.

52. The pharmaceutical formulation of any one of claims 1-16, 18-35, and 37-46, wherein the at least one PEGylated lipid is DPG-PEG2000 present at from about 0.2 to about 0.5 mg/mL in the pharmaceutical formulation.

53. The pharmaceutical formulation of claim 52, wherein the DPG-PEG2000 is present at about 0.25 mg/mL in the pharmaceutical formulation.

54. The pharmaceutical formulation of any one of claims 1-19, 21-37, and 39-53, wherein the at least one surfactant is PEG400 present at from about 0.75 to about 6 mg/mL in the pharmaceutical formulation.

55. The pharmaceutical formulation of claim 54, wherein the PEG400 is present at from about 1.5 to about 3 mg/mL in the pharmaceutical formulation.

56. The pharmaceutical formulation of claim 54 or 55, wherein the PEG400 is present at about 3 mg/mL in the pharmaceutical formulation.

57. The pharmaceutical formulation of any one of claims 1-19, 21-37, and 39-53, wherein the at least one surfactant is PEG1000 present at from about 0.75 to about 3 mg/mL in the pharmaceutical formulation.

58. The pharmaceutical formulation of claim 57, wherein the PEG1000 is present at about 3 mg/mL in the pharmaceutical formulation.

59. The pharmaceutical formulation of any one of claims 1-19, 21-37, and 39-53, wherein the at least one surfactant is propylene glycol present at from about 0.75 to about 3 mg/mL in the pharmaceutical formulation.

60. The pharmaceutical formulation of claim 59, wherein the propylene glycol is present at about 1.5 mg/mL in the pharmaceutical formulation.

61. The pharmaceutical formulation of any one of claims 1-26, 28-41, and 43-60, wherein the at least one alcohol cosolvent is ethanol present at from about 3% to about 10% (w/w) based on the total weight of the pharmaceutical formulation in the pharmaceutical formulation.

62. The pharmaceutical formulation of claim 61, wherein the ethanol is present at from about 3% to about 5% (w/w) based on the total weight of the pharmaceutical formulation in the pharmaceutical formulation.

63. The pharmaceutical formulation of any one of claims 1-26, 28-41, and 43-60, wherein the at least one alcohol cosolvent is isopropyl alcohol present at from about 5% to about 10% (w/w) based on the total weight of the pharmaceutical formulation in the pharmaceutical formulation.

64. The pharmaceutical formulation of claim 63, wherein the isopropyl alcohol is present at about 10% (w/w) based on the total weight of the pharmaceutical formulation in the pharmaceutical formulation.

65. The pharmaceutical formulation of any one of claims 1-23, 25-39, and 41-64, wherein the at least one hydrofluoroalkane propellant is HFA134a.

66. The pharmaceutical formulation of any one of claims 1-23, 25-39, and 41-64, wherein the at least one hydrofluoroalkane propellant is HFA227ea.

67. The pharmaceutical formulation of any one of claims 1-23, 25-39, and 41-64, wherein the at least one hydrofluoroalkane propellant is HFA152a.

68. The pharmaceutical formulation of any one of claims 1-67, wherein R₂ is linear dodecyl, linear tridecyl, linear tetradecyl, linear pentadecyl, linear hexadecyl, linear heptadecyl or linear octadecyl.

69. The pharmaceutical formulation of any one of claims 1-68, wherein R₂ is linear dodecyl.

70. The pharmaceutical formulation of any one of claims 1-68, wherein R₂ is linear tridecyl.

71. The pharmaceutical formulation of any one of claims 1-68, wherein R₂ is linear tetradecyl.

72. The pharmaceutical formulation of any one of claims 1-68, wherein R₂ is linear pentadecyl.

73. The pharmaceutical formulation of any one of claims 1-68, wherein R₂ is linear hexadecyl.

74. The pharmaceutical formulation of claim 73, wherein the compound of Formula (Ia) or (II), or an enantiomer, diastereomer, or pharmaceutically acceptable salt thereof is a compound of Formula (III): 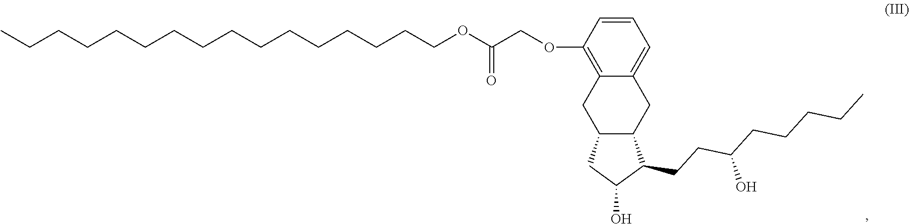 [SMILES: CCCCCCCCCCCCCCCCOC(=O)COc1cccc2c1C[C@H]1C[C@@H](O)[C@H](CC[C@@H](O)CCCCC)[C@H]1C2]
or an enantiomer, diastereomer, or pharmaceutically acceptable salt thereof.

75. The pharmaceutical formulation of claim 74, wherein the compound of Formula (Ia) or (II), or pharmaceutically acceptable salt thereof is a compound of Formula (III) or a pharmaceutically acceptable salt thereof.

76. The pharmaceutical formulation of claim 74 or 75, wherein the compound of Formula (Ia) or (II), or pharmaceutically acceptable salt thereof is a compound of Formula (III).

77. The pharmaceutical formulation of any one of claims 1-68, wherein R₂ is linear heptadecyl.

78. The pharmaceutical formulation of any one of claims 1-68, wherein R₂ is linear octadecyl.

79. The pharmaceutical formulation of any one of claims 1-78, wherein (b) is polyoxyethylene (20) cetylether.

80. The pharmaceutical formulation of any one of claims 1-78, wherein (b) is at least one polyethylene glycol-lipid (PEGylated lipid).

81. The pharmaceutical formulation of claim 29, wherein R₂ is linear hexadecyl and the compound of Formula (II), or an enantiomer, diastereomer, or pharmaceutically acceptable salt thereof is a compound of Formula (III): 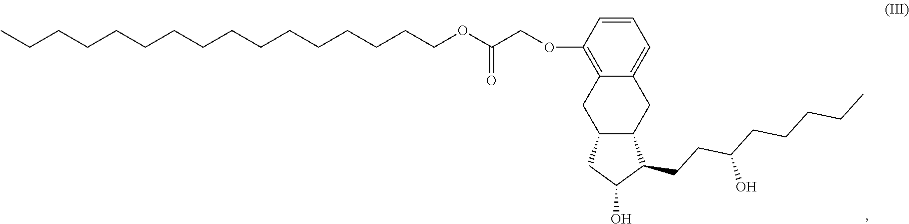 [SMILES: CCCCCCCCCCCCCCCCOC(=O)COc1cccc2c1C[C@H]1C[C@@H](O)[C@H](CC[C@@H](O)CCCCC)[C@H]1C2]
or an enantiomer, diastereomer, or pharmaceutically acceptable salt thereof, and wherein the pharmaceutical formulation comprises 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a.

82. The pharmaceutical formulation of claim 81, wherein R₂ is linear hexadecyl and the compound of Formula (II) or pharmaceutically acceptable salt thereof is a compound of Formula (III) or a pharmaceutically acceptable salt thereof, and wherein the pharmaceutical formulation comprises 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a.

83. The pharmaceutical formulation of claim 81 or 82, wherein R₂ is linear hexadecyl and the compound of Formula (II) or pharmaceutically acceptable salt thereof is a compound of Formula (III), and wherein the pharmaceutical formulation comprises 1 mg/mL of the compound of Formula (III),0.5 mg/mL DSPE-PEG2000, 3 mg/mL PEG400, 10% (w/w) isopropyl alcohol based on the total weight of the pharmaceutical formulation, and HFA134a.

84. The pharmaceutical formulation of claim 32, wherein R₂ is linear hexadecyl and the compound of Formula (II), or an enantiomer, diastereomer, or pharmaceutically acceptable salt thereof is a compound of Formula (III): 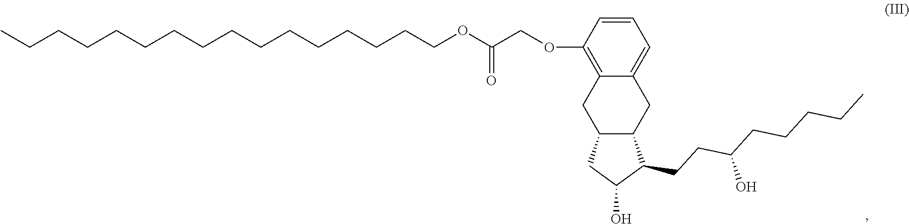 [SMILES: CCCCCCCCCCCCCCCCOC(=O)COc1cccc2c1C[C@H]1C[C@@H](O)[C@H](CC[C@@H](O)CCCCC)[C@H]1C2]
or an enantiomer, diastereomer, or pharmaceutically acceptable salt thereof, and wherein the pharmaceutical formulation consists of 1 mg/mL of the compound of Formula (III), or an enantiomer, diastereomer, or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a.

85. The pharmaceutical formulation of claim 84, wherein R₂ is linear hexadecyl and the compound of Formula (II) or pharmaceutically acceptable salt thereof is a compound of Formula (III) or a pharmaceutically acceptable salt thereof, and wherein the pharmaceutical formulation consists of 1 mg/mL of the compound of Formula (III) or pharmaceutically acceptable salt thereof, 0.5 mg/mL DSPE-PEG2000, 3 mg/mL PEG400, 10% (w/w) isopropyl alcohol based on the total weight of the pharmaceutical formulation, and HFA134a.

86. The pharmaceutical formulation of claim 84 or 85, wherein R₂ is linear hexadecyl and the compound of Formula (II) or pharmaceutically acceptable salt thereof is a compound of Formula (III), and wherein the pharmaceutical formulation consists of 1 mg/mL of the compound of Formula (III), 0.5 mg/mL DSPE-PEG2000, 3 mg/mL PEG400, 10% (w/w) isopropyl alcohol based on the total weight of the pharmaceutical formulation, and HFA134a.

87. The pharmaceutical formulation of any one of claims 1-86, wherein the pharmaceutical formulation is in the form of an aerosol.

88. The pharmaceutical formulation of claim 87, wherein the aerosol has a mass median aerodynamic diameter (MMAD) of from about 1 to about 3 am, as measured by Next Generation Impactor (NGI).

89. The pharmaceutical formulation of claim 87, wherein the aerosol has an MMAD of from about 1 to about 2 pam, as measured by NG.

90. The pharmaceutical formulation of claim 87, wherein the aerosol has an MMAD of about 1.5 μm, as measured by NGI.

91. The pharmaceutical formulation of any one of claims 87-90, wherein the aerosol has a throat deposition of from about 5% to about 40%, as measured by NGI.

92. The pharmaceutical formulation of any one of claims 87-90, wherein the aerosol has a throat deposition of from about 10% to about 30%, as measured by NGI.

93. The pharmaceutical formulation of any one of claims 87-90, wherein the aerosol has a throat deposition of from about 10% to about 25%, as measured by NGI.

94. The pharmaceutical formulation of any one of claims 87-90, wherein the aerosol has a throat deposition of from about 15% to about 25%, as measured by NGI.

95. The pharmaceutical formulation of any one of claims 87-90, wherein the aerosol has a throat deposition of from about 15% to about 20%, as measured by NCI.

96. The pharmaceutical formulation of any one of claims 87-95, wherein the aerosol has a fine particle fraction (FPF) of from about 50% to about 95%, as measured by NGI.

97. The pharmaceutical formulation of any one of claims 87-95, wherein the aerosol has an FPF of from about 60% to about 85%, as measured by NGI.

98. The pharmaceutical formulation of any one of claims 87-95, wherein the aerosol has an FPF of from about 70% to about 85%, as measured by NGI.

99. The pharmaceutical formulation of any one of claims 87-95, wherein the aerosol has an FPF of from about 75% to about 85%, as measured by NGI.

100. The pharmaceutical formulation of any one of claims 87-95, wherein the aerosol has an FPF of from about 70% to about 80%, as measured by NGI.

101. A canister comprising a metering valve and the pharmaceutical formulation of any one of claims 1-100.

102. The canister of claim 101, which comprises an aluminum can which is anodized, lacquer-coated and/or plastic coated.

103. The canister of claim 102, wherein the aluminum can is coated with a fluorocarbon polymer.

104. The canister of claim 103, wherein the fluorocarbon polymer is selected from fluorinated ethylene propylene, polytetrafluoroethylene, or a combination thereof.

105. A metered dose inhaler (MDI) comprising the canister of any one of claims 101-104 fitted into a suitable channeling device.

106. The MDI of claim 105, wherein the channeling device comprises a mouthpiece actuator having an actuator orifice of from about 0.1 to about 0.3 mm in diameter.

107. The MDI of claim 105 or 106, which is configured to have an emitted dose of from about 30 to about 70 μg of the compound of Formula (Ia), (Ib), (II), or (III), or an enantiomer, diastereomer, or pharmaceutically acceptable salt thereof.

108. The MDI of claim 107, which is configured to have an emitted dose of from about 30 to about 70 μg of the compound of Formula (Ia), (Ib), (II), or (III), or pharmaceutically acceptable salt thereof.

109. The MDI of claim 108, which is configured to have an emitted dose of from about 30 to about 70 μg of the compound of Formula (Ia), (Ib), (II), or (III).

110. The MDI of claim 105 or 106, which is configured to have an emitted dose of from about 40 to about 60 μg of the compound of Formula (Ia), (Ib), (II), or (III), or an enantiomer, diastereomer, or pharmaceutically acceptable salt thereof.

111. The MDI of claim 110, which is configured to have an emitted dose of from about 40 to about 60 μg of the compound of Formula (Ia), (Ib), (II), or (III), or pharmaceutically acceptable salt thereof.

112. The MDI of claim 111, which is configured to have an emitted dose of from about 40 to about 60 μg of the compound of Formula (Ia), (Ib), (II), or (III).

113. The MDI of any one of claims 105-112, wherein the MDI is configured to produce an aerosol of the pharmaceutical formulation with an MMAD of from about 1 to about 3 μm, as measured by NGI.

114. The MDI of any one of claims 105-112, wherein the MDI is configured to produce an aerosol of the pharmaceutical formulation with an MMAD of from about 1 to about 2 μm, as measured by NGI.

115. The MDI of any one of claims 105-112, wherein the MDI is configured to produce an aerosol of the pharmaceutical formulation with an MMAD of about 1.5 μm, as measured by NGI.

116. The MDI of any one of claims 105-115, wherein the MDI is configured to produce an aerosol of the pharmaceutical formulation with a throat deposition of from about 5% to about 40%, as measured by NGI.

117. The MDI of any one of claims 105-115, wherein the MDI is configured to produce an aerosol of the pharmaceutical formulation with a throat deposition of from about 10% to about 30%, as measured by NGI.

118. The MDI of any one of claims 105-115, wherein the MDI is configured to produce an aerosol of the pharmaceutical formulation with a throat deposition of from about 10% to about 25%, as measured by NGI.

119. The MDI of any one of claims 105-115, wherein the MDI is configured to produce an aerosol of the pharmaceutical formulation with a throat deposition of from about 15% to about 25%, as measured by NGI.

120. The MDI of any one of claims 105-115, wherein the MDI is configured to produce an aerosol of the pharmaceutical formulation with a throat deposition of from about 15% to about 20%, as measured by NGI.

121. The MDI of any one of claims 105-120, wherein the MDI is configured to produce an aerosol of the pharmaceutical formulation with a fine particle fraction (FPF) of from about 50% to about 95%, as measured by NGI.

122. The MDI of any one of claims 105-120, wherein the MDI is configured to produce an aerosol of the pharmaceutical formulation with an FPF of from about 60% to about 85%, as measured by NGI.

123. The MDI of any one of claims 105-120, wherein the MDI is configured to produce an aerosol of the pharmaceutical formulation with an FPF of from about 70% to about 85%, as measured by NC.

124. The MDI of any one of claims 105-120, wherein the MDI is configured to produce an aerosol of the pharmaceutical formulation with an FPF of from about 75% to about 85%, as measured by NGI.

125. The MDI of any one of claims 105-120, wherein the MDI is configured to produce an aerosol of the pharmaceutical formulation with an FPF of from about 70% to about 80%, as measured by NGI.

126. The MDI of any one of claims 105-125, wherein the MDI is configured to produce an aerosol of the pharmaceutical formulation with a fine particle dose (FPD) of from about 10 to about 40 μg, as measured by NGI.

127. The MDI of any one of claims 105-125, wherein the MDI is configured to produce an aerosol of the pharmaceutical formulation with an FPD of from about 15 to about 40 μg, as measured by NGI.

128. The MDI of any one of claims 105-125, wherein the MDI is configured to produce an aerosol of the pharmaceutical formulation with an FPD of from about 20 to about 40 μg, as measured by NGI.

129. The MDI of any one of claims 105-125, wherein the MDI is configured to produce an aerosol of the pharmaceutical formulation with an FPD of from about 30 to about 40 μg, as measured by NGI.

130. The MDI of any one of claims 105-125, wherein the MDI is configured to produce an aerosol of the pharmaceutical formulation with an FPD of from about 30 to about 35 μg, as measured by NGI.

131. A method for treating pulmonary hypertension in a patient in need thereof, comprising administering an effective amount of the pharmaceutical formulation of any one of claims 1-100 to the lungs of the patient by inhalation via a metered dose inhaler.

132. The method of claim 131, wherein the pulmonary hypertension is pulmonary arterial hypertension.

133. The method of claim 132, wherein the pulmonary arterial hypertension is class I pulmonary arterial hypertension, as characterized by the New York Heart Association (NYHA).

134. The method of claim 132, wherein the pulmonary arterial hypertension is class II pulmonary arterial hypertension, as characterized by the NYHA.

135. The method of claim 132, wherein the pulmonary arterial hypertension is class III pulmonary arterial hypertension, as characterized by the NYHA.

136. The method of claim 132, wherein the pulmonary arterial hypertension is class IV pulmonary arterial hypertension, as characterized by the NYHA.

137. The method of claim 131, wherein the pulmonary hypertension is group 1 pulmonary hypertension, as characterized by the World Health Organization (WHO).

138. The method of claim 131, wherein the pulmonary hypertension is group 2 pulmonary hypertension, as characterized by the WHO.

139. The method of claim 131, wherein the pulmonary hypertension is group 3 pulmonary hypertension, as characterized by the WHO.

140. The method of claim 131, wherein the pulmonary hypertension is group 4 pulmonary hypertension, as characterized by the WHO.

141. The method of claim 131, wherein the pulmonary hypertension is group 5 pulmonary hypertension, as characterized by the WHO.

142. A method for treating portopulmonary hypertension or pulmonary fibrosis in a patient in need thereof, comprising administering an effective amount of the pharmaceutical formulation of any one of claims 1-100 to the lungs of the patient by inhalation via a metered dose inhaler.

143. The method of any one of claims 131-142, wherein the administering is conducted in a once-a-day, twice-a-day, or three-times-a-day dosing.

144. The method of any one of claims 131-143, wherein the administering comprises aerosolizing the pharmaceutical formulation and administering an aerosolized pharmaceutical formulation to the lungs of the patient via inhalation.

145. The method of claim 144, wherein the aerosolized pharmaceutical formulation has an MMAD of from about 1 to about 3 μm, as measured by NGI.

146. The method of claim 144, wherein the aerosolized pharmaceutical formulation has an MMAD of from about 1 to about 2 μm, as measured by NGI.

147. The method of claim 144, wherein the aerosolized pharmaceutical formulation has an MMAD of about 1.5 μm, as measured by NGI.

148. The method of any one of claims 144-147, wherein the aerosolized pharmaceutical formulation has a throat deposition of from about 5% to about 40%, as measured by NGI.

149. The method of any one of claims 144-147, wherein the aerosolized pharmaceutical formulation has a throat deposition of from about 10% to about 30%, as measured by NGI.

150. The method of any one of claims 144-147, wherein the aerosolized pharmaceutical formulation has a throat deposition of from about 10% to about 25%, as measured by NGI.

151. The method of any one of claims 144-147, wherein the aerosolized pharmaceutical formulation has a throat deposition of from about 15% to about 25%, as measured by NGI.

152. The method of any one of claims 144-147, wherein the aerosolized pharmaceutical formulation has a throat deposition of from about 15% to about 20%, as measured by NGI.

153. The method of any one of claims 144-152, wherein the aerosolized pharmaceutical formulation has an FPF of from about 50% to about 95%, as measured by NGI.

154. The method of any one of claims 144-152, wherein the aerosolized pharmaceutical formulation has an FPF of from about 60% to about 85%, as measured by NGI.

155. The method of any one of claims 144-152, wherein the aerosolized pharmaceutical formulation has an FPF of from about 70% to about 85%, as measured by NGI.

156. The method of any one of claims 144-152, wherein the aerosolized pharmaceutical formulation has an FPF of from about 75% to about 85%, as measured by NGI.

157. The method of any one of claims 144-152, wherein the aerosolized pharmaceutical formulation has an FPF of from about 70% to about 80%, as measured by NGI.

158. A system for treating pulmonary hypertension, portopulmonary hypertension, or pulmonary fibrosis, comprising:
the pharmaceutical formulation of any one of claims 1-100 and an MDI.

159. The system of claim 158, wherein the MDI comprises a mouthpiece actuator having an actuator orifice of from about 0.1 to about 0.3 mm in diameter.

160. The system of claim 158 or 159, wherein the MDI is configured to have an emitted dose of from about 30 to about 70 μg of the compound of Formula (Ia), (Ib), (II), or (III), or an enantiomer, diastereomer, or pharmaceutically acceptable salt thereof.

161. The system of claim 160, wherein the MDI is configured to have an emitted dose of from about 30 to about 70 μg of the compound of Formula (Ia), (Ib), (II), or (III), or pharmaceutically acceptable salt thereof.

162. The system of claim 160 or 161, wherein the MDI is configured to have an emitted dose of from about 30 to about 70 μg of the compound of Formula (Ia), (Ib), (II), or (III).

163. The system of claim 158 or 159, wherein the MDI is configured to have an emitted dose of from about 40 to about 60 μg of the compound of Formula (Ia), (Ib), (II), or (III), or an enantiomer, diastereomer, or pharmaceutically acceptable salt thereof.

164. The system of claim 163, wherein the MDI is configured to have an emitted dose of from about 40 to about 60 μg of the compound of Formula (Ia), (Ib), (II), or (III), or pharmaceutically acceptable salt thereof.

165. The system of claim 163 or 164, wherein the MDI is configured to have an emitted dose of from about 40 to about 60 μg of the compound of Formula (Ia), (Ib), (II), or (III).

166. The system of any one of claims 158-165, wherein the MDI is configured to produce an aerosol of the pharmaceutical formulation with an MMAD of from about 1 to about 3 μm, as measured by NGI.

167. The system of any one of claims 158-165, wherein the MDI is configured to produce an aerosol of the pharmaceutical formulation with an MMAD of from about 1 to about 2 μm, as measured by NGI.

168. The system of any one of claims 158-165, wherein the MDI is configured to produce an aerosol of the pharmaceutical formulation with an MMAD of about 1.5 μm, as measured by NGI.

169. The system of any one of claims 158-168, wherein the MDI is configured to produce an aerosol of the pharmaceutical formulation with a throat deposition of from about 5% to about 40%, as measured by NGI.

170. The system of any one of claims 158-168, wherein the MDI is configured to produce an aerosol of the pharmaceutical formulation with a throat deposition of from about 10% to about 30%, as measured by NGI.

171. The system of any one of claims 158-168, wherein the MDI is configured to produce an aerosol of the pharmaceutical formulation with a throat deposition of from about 10% to about 25%, as measured by NGI.

172. The system of any one of claims 158-168, wherein the MDI is configured to produce an aerosol of the pharmaceutical formulation with a throat deposition of from about 15% to about 25%, as measured by NGI.

173. The system of any one of claims 158-168, wherein the MDI is configured to produce an aerosol of the pharmaceutical formulation with a throat deposition of from about 15% to about 20%, as measured by NGI.

174. The system of any one of claims 158-173, wherein the MDI is configured to produce an aerosol of the pharmaceutical formulation with an FPF of from about 50% to about 95%, as measured by NGI.

175. The system of any one of claims 158-173, wherein the MDI is configured to produce an aerosol of the pharmaceutical formulation with an FPF of from about 60% to about 85%, as measured by NGI.

176. The system of any one of claims 158-173, wherein the MDI is configured to produce an aerosol of the pharmaceutical formulation with an FPF of from about 70% to about 85%, as measured by NGI.

177. The system of any one of claims 158-173, wherein the MDI is configured to produce an aerosol of the pharmaceutical formulation with an FPF of from about 75% to about 85%, as measured by NGI.

178. The system of any one of claims 158-173, wherein the MDI is configured to produce an aerosol of the pharmaceutical formulation with an FPF of from about 70% to about 80%, as measured by NGI.

179. The system of any one of claims 158-178, wherein the MDI is configured to produce an aerosol of the pharmaceutical formulation with a fine particle dose (FPD) of from about 10 to about 40 μg, as measured by NGI.

180. The system of any one of claims 158-178, wherein the MDI is configured to produce an aerosol of the pharmaceutical formulation with an FPD of from about 15 to about 40 μg, as measured by NGI.

181. The system of any one of claims 158-178, wherein the MDI is configured to produce an aerosol of the pharmaceutical formulation with an FPD of from about 20 to about 40 μg, as measured by NGI.

182. The system of any one of claims 158-178, wherein the MDI is configured to produce an aerosol of the pharmaceutical formulation with an FPD of from about 30 to about 40 μg, as measured by NGI.

183. The system of any one of claims 158-178, wherein the MDI is configured to produce an aerosol of the pharmaceutical formulation with an FPD of from about 30 to about 35 μg, as measured by NGI.
